(19) United States
(12) Patent Application Publication
(10) Pub. No.: US 2025/0249098 A1
(43) Pub. Date: Aug. 7, 2025

(54) CRYOPRESERVATION OF NK CELL PRODUCTS FOR OFF-THE-SHELF IMMUNOTHERAPY

(71) Applicant: BOARD OF REGENTS, THE UNIVERSITY OF TEXAS SYSTEM, Austin, TX (US)

(72) Inventors: Katy REZVANI, Houston, TX (US); Rafet BASAR, Houston, TX (US); Bingqian HU, Houston, TX (US); Emily ENSLEY, Houston, TX (US); Nadima UPRETY, Houston, TX (US)

(73) Assignee: BOARD OF REGENTS, THE UNIVERSITY OF TEXAS SYSTEM, Austin, TX (US)

(21) Appl. No.: 18/857,092

(22) PCT Filed: May 10, 2023
(86) PCT No.: PCT/US2023/066825

§ 371 Date: Oct. 15, 2024

Related U.S. Application Data
(60) Provisional application No. 63/364,516, filed on May 11, 2022.

(51) Int. Cl. A61K 40/15; A61K 40/31; A61P 35/00; C12N 5/00; C12N 5/0783

(52) U.S. Cl. CPC A61K 40/15; A61K 40/31; A61P 35/00; C12N 5/0646; C12N 5/562; C12N 2501/2302; C12N 2501/2312; C12N 2501/2315; C12N 2501/2318; C12N 2501/727

(57) ABSTRACT

Provided herein are methods for deactivation of effector cells, such as NK cells, including cells for adoptive cell therapy that are off-the-shelf cells. The deactivated cells may be cryopreserved following deactivation. The deactivated and cell may be expanded and/or activated prior to deactivation, and may or may not comprise a transgene, which may optionally encode a chimeric antigen receptor and/or T cell receptor. In certain embodiments, disclosed are methods for deactivation of NK cells comprising treatment with a kinase inhibitor, for example an mTOR inhibitor and/or a tyrosine kinase inhibitor, for example rapamycin and/or Dasatinib. Also provided herein are deactivated and cryopreserved NK cells, and methods of preparing and/or using the same for treatment of a subject in need thereof, for example treatment of a subject with cancer.

### CROSS REFERENCE TO RELATED APPLICATIONS

This application claims priority to U.S. Provisional Patent Application Ser. No. 63/364,516 filed May 11, 2022, which is incorporated by reference herein in its entirety.

### SEQUENCE LISTING

The instant application contains a Sequence Listing which has been submitted in ST26 format and is hereby incorporated by reference in its entirety. Said ST26 copy, created on May 3, 2023, is named MDAC_P1326_Sequence_Listing.xml and is 6,349 bytes in size.

### BACKGROUND

### 1. Technical Field

The present disclosure relates generally at least to the fields of cell biology, molecular biology, biochemistry, immunology, and medicine.

### 2. Description of Related Art

Culture and storage of cells, e.g., mammalian cells, for in vitro studies or ex vivo culture and/or storage for later administration to a human or animal is an important tool for the study and treatment of human diseases. Cell culture and storage is widely used for the production of various biologically active products, such as viral vaccines, monoclonal antibodies, polypeptide growth factors, hormones, enzymes and tumor specific antigens. However, many of the media or methods used to culture and/or store the cells comprise components that can have negative effects on cell growth and/or maintenance of cells in culture, and/or are not sufficient to protect the cells from the consequences of storage (e.g., cryopreservation).

In addition, presently several cell banks exist that store cells, for example human placental or umbilical cord stem cells, for future medical use. There are also cell banks that store cells, cultivated in for example bioreactors, for scientific purposes as well as for medical therapies. Common for all cell banks is that the cells are stored by cryopreservation usually in liquid nitrogen. The cryopreservation of cells using current methodologies can have a detrimental effect on cell function and/or viability.

Current FDA-approved CAR T-cells are autologous and manufactured in a highly personalized way for each patient, while this method has proven efficacious, it is a time consuming and costly endeavor. The development of readily available off-the-shelf allogeneic products provides the opportunity to generate cell banks in advance that can be used as needed. An off-the-shelf effector cell product (e.g., NK cell, CAR-NK cell, etc.) must satisfy two conditions: (i) the donor cells must be able to be infused without consideration for HLA matching, and (ii) the donor cells must preserve functionality after cryopreservation. The immediate inventors have previously shown that subjects can be safely infused with CAR19/IL-15 cord blood (CB) derived NK cells (see e.g., Liu E, Marin D, Banerjee P, et al. Use of CAR-Transduced Natural Killer Cells in CD19-Positive Lymphoid Tumors. N Engl J Med. 2020; 382(6):545-553, which is incorporated herein for the purposes described herein) that are HLA mismatched with the recipient, which addresses the first requirement for an off-the-shelf product. However, as opposed to T cells, the cytotoxicity of which is not significantly affected by freeze-thaw cycles, the ability of NK cells to expand, persist, home to sites of disease and/or kill tumor cells in vivo can be deeply impacted by cryopreservation (see e.g., Mark C, Czerwinski T, Roessner S, et al., Cryopreservation impairs 3-D migration and cytotoxicity of natural killer cells. Nat Commun. 2020; 11(1):5224; and Miller J S, Rooney C M, Curtsinger J, et al. Expansion and homing of adoptively transferred human natural killer cells in immunodeficient mice varies with product preparation and in vivo cytokine administration: implications for clinical therapy. Biol Blood Marrow Transplant. 2014; 20(8):1252-1257; each of which is incorporated herein for the purposes described herein). Due to this limitation, clinical trials testing the safety and efficacy of NK cell therapies often must utilize fresh post-expansion NK cells.

The present disclosure satisfies a need in the art for improved cell storage methods.

### BRIEF SUMMARY

The present disclosure concerns cells, deactivation, and/or cryopreservation techniques, and methods of utilizing cells and/or compositions produced as described herein. In general, the present disclosure is related to immune cells, such as NK cells, which can be deactivated using one or more deactivation agents, cryopreserved, and subsequently thawed as described herein. These cells are more robust in survival capacity and/or cytotoxicity against tumor cells when compared to cells cryopreserved in the absence of the disclosed methods. In particular embodiments, the cell deactivation methods described herein halts the cytolytic activity, cytokine production, and/or proliferation of NK cells, allowing for enhanced cell viability following cryopreservation, and in specific embodiments the generation of cells that are to be used “off-the-shelf.” Following cell deactivation and cryopreservation, the cells upon thawing may be used immediately, expanded, or may be further manipulated, such as subject to recombination techniques including transfection, for example. In some cases the cells are cryopreserved a second or subsequent time, whether or not utilizing the disclosed methodologies, and prior to the second or subsequent cryopreservation, the cells may or may not be further manipulated, such as subject to recombination techniques including but not limited to, transduction, transfection, and/or gene editing, etc.

In some embodiments, disclosed herein are methods of deactivating a Natural Killer (NK) cell, comprising treating an NK cell with an effective amount of one or more deactivating agents under conditions to produce a deactivated NK cell. In some embodiments, a deactivating agent is a kinase inhibitor. In some embodiments, a deactivating agent is a mechanistic target of rapamycin (mTOR) inhibitor. In some embodiments, the mTOR inhibitor is rapamycin, everolimus, and/or temsirolimus. In some embodiments, the mTOR inhibitor is rapamycin. In some embodiments, the deactivating agent is a tyrosine kinase (TK) inhibitor. In some embodiments, the TK inhibitor is Dasatinib, Nilotinib, Lorlatinib, Brigatinib, Ceritinib, Alectinib, Crizotinib, Bosutinib, Ponatinib, Saracatinib, Imatinib, Zanubrutinib, Acalabrutinib, Ibrutinib, Capmatinib, Pexidartinib, Dacomitinib, Osimertinib, Erlotinib, Gefitinib, Lapatinib, Afatinib, Pemigatinib, Erdafitinib, Nintedanib, Gilteritinib, Midostaurin, Tucatinib, Neratinib, Baricitinib, Ruxolitinib, Fedratinib, Tofacitinib, Ripretinib, Selumetinib, Binimetinib, Cobimetinib, Trametinib, Upadacitinib, Avapritinib, Selpercatinib, Cabozantinib, Fostamatinib, Larotrectinib, Entrectinib, Axitinib, Regorafenib, Pazopanib, Sorafenib, Lenvatinib, Vandetanib, and/or Sunitinib. In some embodiments, the TK inhibitor is a BCR-Abl inhibitor. In some embodiments, the TK inhibitor is Dasatinib, Nilotinib, Bosutinib, Ponatinib, and/or Imatinib. In some embodiments, the TK inhibitor is Dasatinib and/or Nilotinib. In some embodiments, the TK inhibitor is Dasatinib. In some embodiments, an NK cell is not treated with Bosutinib. In some embodiments, an NK cell is not treated with Nilotinib. In some embodiments, an NK cell is not treated with Saracatinib. In some embodiments, an NK cell is not treated with an mTOR inhibitor.

In some embodiments, treatment with a deactivating agent is at any point during culturing of the NK cell. In some embodiments, the treatment is for about 24 to about 96 hours, about 36 to about 84 hours, or about 48 to about 72 hours. In some embodiments, the treatment is for about 24 hours, about 48 hours, or about 72 hours. In some embodiments, the NK cell is treated with the deactivating agent at a concentration of about 1 to about 1000 nM. In some embodiments, the NK cell is treated with the deactivating agent at a concentration of about 5 to about 500 nM. In some embodiments, the NK cell is treated with the deactivating agent at a concentration of about 20 to about 200 nM. In some embodiments, the NK cell is treated with the deactivating agent at a concentration of about 30 to about 100 nM. In some embodiments, the deactivated NK cell has an increased expression of one or more of C-kit, CCR-5, CD62L and/or CXCR4, and/or decreased expression of one or more of NKG2D, DNAM, OX-40, TRAIL, HLA-DR, CD2, CD25, ICOS, and/or CD95 relative to an activated NK cell.

In some embodiments, the NK cell is derived from human peripheral blood mononuclear cells (PBMC), unstimulated leukapheresis products (PBSC), human embryonic stem cells (hESCs), hematopoietic stem cells, induced pluripotent stem cells (iPSCs), bone marrow, an NK cell line, and/or umbilical cord blood. In some embodiments, the NK cell is isolated from blood. In some embodiments, the NK cell is isolated from one or more umbilical cord blood units. In some embodiments, the NK cell is an induced NK cell created from a precursor cell. In some embodiments, the precursor cell is a hESC, hematopoietic stem cell, iPSC, and/or induced hematopoietic stem cell.

In some embodiments, the NK cell comprises a transgene. In some embodiments, the transgene encodes a chimeric antigen receptor (CAR), a T-cell receptor (TCR), a non-naturally occurring variant of FcγRIII (CD16), an interleukin (e.g., interleukin 15 (IL-15), interleukin 15 receptor (IL-15R) or a variant thereof, interleukin 12 (IL-12), interleukin 21 (IL-21), interleukin 18 (IL-18), interleukin-12 receptor (IL-12R) or a variant thereof), a human leukocyte antigen (e.g., human leukocyte antigen G (HLA-G), human leukocyte antigen E (HLA-E)), leukocyte surface antigen cluster of differentiation CD47 (CD47), or any combination of two or more thereof. In some embodiments, the NK cell comprises a transgenic CAR. In some embodiments, the NK cell comprises more than one transgenic CAR.

In some embodiments, the NK cell comprises a mutation in an endogenous gene. In some embodiments, the endogenous gene is an immunomodulatory gene. In some embodiments, the endogenous gene is NKG2A, SIGLEC-7, LAG3, TIM3, CISH, FOXOl, TGFBR2, TIGIT, CD96, ADORA2, NR3C1, PD1, PDL-1, PDL-2, CD47, SIRPA, SHIP1, ADAM 17, RPS6, 4EBP1, CD25, CD40, IL21R, ICAM1, CD95, CD80, CD86, IL10R, TDAG8, CD5, CD7, SLAMF7, CD38, LAG3, TCR, beta2-microglobulin, HLA, CD73, GCR, CREM, ICER, CREB1, and/or CD39.

In some embodiments, the NK cell is activated and/or expanded prior to deactivation. In some embodiments, the NK cells are activated and/or expanded by culturing with a cell culture solution comprising universal Antigen Presenting Cells (uAPC), IL-2, IL-12, IL-15, and/or IL-18.

In some embodiments, the deactivated NK cells are frozen and cryopreserved for any period of time. In some embodiments, the deactivating agent is included in the cryopreservation media. In some embodiments, the deactivating agent is washed off of the deactivated NK cells prior to cryopreservation.

Also provided herein, in some embodiments, are NK cells produced by any one or any combination of the methods described herein. In some embodiments, provided herein are cryopreserved deactivated NK cell produced by any one or more of the methods described herein. In some embodiments, the cryopreserved deactivated NK cell is thawed to produce a thawed deactivated NK cell. In some embodiments, the thawed deactivated NK cell is washed to remove the deactivating agent. In some embodiments, the thawed deactivated NK cell is reactivated in the absence of a deactivating agent, producing a reactivated NK cell. In some embodiments, the reactivated NK cell has improved survival rates relative to a non-deactivated thawed cryopreserved NK cell. In some embodiments, the reactivated NK cell comprises a transgene, the transgene expression levels are not significantly decreased relative to a non-deactivated thawed cryopreserved NK cell. In some embodiments, the transgene expression levels are increased relative to a non-deactivated thawed cryopreserved NK cell. In some embodiments, the reactivated NK cell has increased tumor cell killing rates following cryopreservation relative to a non-deactivated thawed cryopreserved NK cell.

Also provided herein, in some embodiments, are methods of treating a subject in need thereof, comprising the step of administering to the subject a therapeutically effective amount of the reactivated NK cells described herein. In some embodiments, the subject has cancer. In some embodiments, the cancer is hematological. In some embodiments, the cancer comprises a solid tumor. In some embodiments, the cancer is of hematopoietic origin. In some embodiments, the reactivated NK cells are allogeneic or autologous with respect to the subject. In some embodiments, the reactivated NK cells are allogeneic with respect to the subject. In some embodiments, the subject has an improved probability of survival relative to a subject not treated with an effective dose of a reactivated NK cell.

Embodiments of the disclosure include methods of maintaining the viability of a population of cells over at least 50% percent following cryopreservation of the population, comprising the step of subjecting the population to an effective amount of one or more deactivating agents (e.g., a kinase inhibitor) to deactivate the cells prior to cryopreservation, cryopreserving the cells, and thawing the population, wherein upon thawing the viability of the population is over at least 50%. In some cases, upon thawing of the cells the viability of the population of cells is over at least 55, 60, 65, 70, 75, 80, 85, 90, 91, 92, 93, 94, 95, 96, 97, 98, or 99% following cryopreservation of the population.

Methods of prolonging the shelf life of a population of cells (for example, effector cells such as NK cells) upon cryopreservation of the population are contemplated herein, such as comprising the step of subjecting the population to an effective amount of a deactivating agent (e.g., a kinase inhibitor) prior to cryopreservation. In some embodiments, the shelf life may be prolonged on the order of 1-4, 1-2, 1-3, 2-4, 2-3, or 3-4 weeks, 1-12, 2-12, 3-12, 4-12, 5-12, 6-12, 7-12, 8-12, 9-12, 10-12, or 11-12, months, or 1, 2, 3, 4, 5, 6, 7, 8, 9, 10, 11, 12, 13, 14, 15, 16, 17, 18, 19, 20, or more years compared to shelf life of cryopreserved cells in the absence of a deactivating agent (e.g., a kinase inhibitor) prior to cryopreservation. The cells may or may not comprise one or more transgenes, such as but not limited to chimeric antigen receptors (CARs), engineered cytokines, and/or T-cell receptors (TCRs). In certain embodiments, the cells may be combined with mono, bispecific, and/or multispecific antibodies either in vivo or ex vivo. Following cryopreservation and thawing of the cells, an effective amount of the cells may be delivered to a subject in need thereof. The cells may be allogeneic or autologous with respect to the recipient subject, and the subject may have cancer, autoimmune disorder, graft versus host disease, allograft rejection, and/or an inflammatory condition, including a bacterial, viral, or fungal infection. In some embodiments, the subject may also have vital organ damage in need of regenerative repair.

Embodiments of the disclosure include methods of thawing a population of cells that have been cryopreserved with any method disclosed herein, such as comprising the steps of exposing the population of cells to an effective amount of a deactivating agent (e.g., a kinase inhibitor) prior to cryopreservation to produce a population of deactivated cells, cryopreserving the cells, and exposing the cryopreserved population to suitable thawing conditions. The thawing conditions may or may not be standard in the art. For example, in specific embodiments one may thaw frozen cells rapidly (<1 minute), such as in a 37° C. water bath and this may be followed by diluting the thawed cells, including slowly, and optionally using pre-warmed growth medium. In specific cases, thawed cells are plated at high density to optimize recovery.

Certain embodiments of the disclosure concern methods of delivering cells to a target site or tissue in an individual, comprising the step of infusing or administering an effective amount of the cells intravenously, locally, intrathecally, intraperitoneally, subcutaneously to the target site or tissue substantially immediately and/or substantially directly following thawing of the cells, including wherein the cells were cryopreserved in the cryopreservation medium composition of the disclosure. In specific embodiments, the target site or tissue is cancerous, such as being a solid tumor, although hematological malignancies also may be treated in methods of the disclosure.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present disclosure. The subject matter of the disclosure may be better understood by reference to one or more of these drawings in combination with the detailed description of specific embodiments presented herein.

FIG. 1 is a schematic diagram describing the strategy of NK cell activation/expansion followed by deactivation via treatment with a deactivating agent (e.g., a kinase inhibitor, e.g., a tyrosine kinase (TK) inhibitor, e.g., Dasatinib, Nilotinib, Imatinib, bosutinib) or an mTOR inhibitor for 24, 48 or 72 hours (hr) in preparation for storage (e.g., cryopreservation). NK cells are isolated and/or derived from a suitable source (e.g., blood, e.g., cord blood), optionally pre-activated using a cytokine cocktail (e.g., IL-12, Il-15, and/or IL-18), optionally expanded and/or activated (e.g., by inclusion of universal Antigen Presenting Cells (APCs) and/or IL-2), and once expanded and/or activated, NK cells are temporarily deactivated (e.g., induced into a resting state) by treating with a deactivating agent, after a set period of time the deactivating agent is removed (e.g., by washing) and cells can then be stored (e.g., by cryopreservation).

FIGS. 2A-2B show how addition of Dasatinib prior to cryopreservation switched the NK cell phenotype to a less activated state when analyzed using mass cytometry. On the left side of FIGS. 2 (A) and (B) is a t-distributed stochastic neighbor embedding (t-SNE) plot showing a clear delineation in phenotypes between activated NK cells treated with Dasatinib, and those not treated with Dasatinib. (A) displays the relative expression of activation and cytotoxicity markers such a CD95, NKG2D, DNAM, OX-40, TRAIL, HLA-DR, CD2, CD25, and ICOS, which displayed increased expression in NK cells not treated with Dasatinib when compared to NK cells treated with Dasatinib. (B) displays the relative expression of activation and cytotoxicity markers such a CCR5, CD62L, CXCR4, and C-kit which displayed decreased expression in NK cells not treated with Dasatinib when compared to NK cells treated with Dasatinib.

FIGS. 3A-3B show how addition of Dasatinib pre-cryopreservation improved NK cell viability post thaw without negatively affecting CAR expression. (A) displays flow cytometry results depicting how NK cells treated with Dasatinib for 24, 48 or 72 hours (h) prior to cryopreservation displayed comparable and/or improved viability relative to NK cells not treated with Dasatinib prior to cryopreservation. The Y axis quantifies Annexin V levels (a marker for apoptosis), while the X axis quantifies a cell death marker. Highlighted and quantified in the bottom left quadrant of each graph display are the living cell percentages (60.2% for no Dasatinib, 61.7% for 24 h Dasatinib, 68.8% for 48 h Dasatinib, and 70.9% for 72 h Dasatinib). (B) displays flow cytometry results depicting how NK cells treated with Dasatinib for 24 or 48 hours prior to cryopreservation displayed comparable and/or improved chimeric antigen receptor (CAR) expression levels relative to NK cells not treated with Dasatinib prior to cryopreservation. The Y axis quantifies CD56 levels, while the X axis quantifies CAR levels. The percentage of cells that are CAR positive and the Mean Fluorescence Intensity (MFI) is displayed for each test condition (57% CAR+ for no Dasatinib with MFI of 3724, 64.9% CAR+ for 24 h Dasatinib with MFI of 7987, and 55.4% CAR+ for 48 h Dasatinib with MFI of 4620).

FIGS. 4A-4B show how addition of Dasatinib for 48 or 72 hours pre-cryopreservation improved NK cell viability and anti-tumor cytotoxicity post thaw. (A) is a graph showing NK cell death following cryopreservation and subsequent thawing. Cytotox green dye was used to measure NK cell death, and green total integrated intensity was used as a surrogate for NK cell death (Y axis) over time (X axis). This data showed that addition of Dasatinib to the culture of NK cells for 48 or 72 hours prior to cryopreservation enhanced NK cell viability post-thaw. (B) is a graph showing tumor cell death following cryopreservation and subsequent thawing of NK cells that are co-cultured at an effector target ratio of 1:1 with the tumor cells (e.g., Raji tumor cells). Cytotox green dye was used to measure tumor cell death, and green total integrated intensity was used as a surrogate for tumor cell death (Y axis) over time (X axis) after addition of NK cells. This showed that the addition of Dasatinib to the culture of NK cells for 48 hr or 72 hrs prior to cryopreservation enhanced their anti-tumor potential.

FIGS. 5A-5B show how addition of Dasatinib for 48 or 72 hours pre-cryopreservation improved NK cell anti-tumor cytotoxicity post thaw. (A) is a graph showing Karpas cell (e.g., Karpas-299 cell line, a human non-hodgkin's Ki-positive large cell lymphoma cell line) death following co-culturing with NK cells that were deactivated pre-cryopreservation. Cytotox green dye was used to measure Karpas cell death, and green total integrated intensity as a function of Near Infrared Camera (NIR) object area was used as a surrogate for Karpas cell death (Y axis) over time (X axis) after addition of thawed NK cells at an effector target ratio (E:T ratio) of 1:1. (B) is a graph showing Raji cell (e.g., a human B lymphoblastoid cell line) death following co-culturing with NK cells that were deactivated pre-cryopreservation. Cytotox green dye was used to measure Raji cell death, and green total integrated intensity as a function of Near Infrared Camera (NIR) object area was used as a surrogate for Raji cell death (Y axis) overtime (X axis) after addition of NK cells at an effector target ratio (E:T ratio) of 1:1. Together these results demonstrated that addition of Dasatinib to the culture of NK cells prior to cryopreservation enhanced their anti-cancer cell potential.

FIGS. 6A-6B show how addition of Dasatinib pre-cryopreservation improved NK cell anti-tumor cytotoxicity in-vivo and improved survival rates in-vivo in Raji-NSG (e.g., NOD scid gamma genotype) mice. (A) is a graph showing the average radiance of tumors (p/s/cm²/sr, a surrogate for tumor growth) (Y axis) over time (days, X axis) in Raji-NSG mice following Raji tumor cell infusion and concurrent treatment with approximately 1×10⁷ thawed NK cells. The results showed that NK cells treated with Dasatinib for 24 h, 48 h, or 72 h pre-cryopreservation displayed improved inhibition of tumor growth relative to mice treated with thawed NK cells that were not treated with Dasatinib pre-cryopreservation or mice with tumors that were not treated with NK cells. (B) is a graph showing the probability of survival (Y axis) of Raji-NSG mice infused with Raji tumor cells and NK cells (approximately 1×10⁷ thawed NK cells) or control media over time (X axis). The results showed that thawed NK cells treated with Dasatinib for 24 h, 48 h, or 72 h pre-cryopreservation displayed improved inhibition of tumor growth relative to mice treated with thawed NK cells that were not treated with Dasatinib or mice with tumors that were not treated with NK cells.

FIGS. 7A-7D show how addition of Dasatinib pre-cryopreservation improved NK cell anti-tumor cytotoxicity and NK cell engraftment in vivo. (A) is a schematic outlining the experimental procedure performed (animals were irradiated on day −4; injected with 0.5×10⁶ MM1S cells on day −3; injected with 5×10⁶ fresh CAR NK cells or 10×10⁶ frozen CAR NK cells on day 0; and imaged weekly throughout life). (B) displays bioluminescent imaging over time (day 0, day 7, day 14, and day 21) for the mice engrafted with MM1s cells (CD70+ multiple myeloma cells) transduced with FireFlyluciferase (FFluc) with no treatment (MM1S alone), fresh day 15 NK cells transduced with a construct comprising a CD70 CAR and IL-15, thawed NK cells comprising the same construct that were treated with Dasatinib for 24 hrs or 72 hrs pre-cryopreservation and frozen on day 15 or on day 18 respectively, or NK cells comprising the same construct that were cryopreserved on day 18 and thawed without pre-cryopreservation Dasatinib treatment. (C) is a graphical quantification of the bioluminescence average radiance displayed in (B). (D) is a graphical quantification of the percentage of in vivo NK cell engraftment on day 10 following NK cell infusion into the mice displayed in (B).

FIGS. 8A-8B demonstrates that CAR NK cells cultured for 14 days and then treated with Dasatinib for 24 or 72 hours prior to freezing were superior in terms of in vivo tumor control when compared to CAR NK cells stored with standard freezing media protocols without deactivation. Furthermore, the Dasatinib treated cells demonstrated similar in vivo anti-tumor control compared to that of fresh CAR NK cells (cultured for 14 days). (A) Bioluminescence imaging (BLI) showing MM1S (CD70+ multiple myeloma cells, transduced with FireFlyluciferase (FFluc)) tumor growth over time (day −3, day 4, day 15, day 22, and day 29) in mice that received MM1S cells alone, MM1S cells plus Dasatinib, MM1S cells plus fresh (day 14 of culture) CD70-targeting CAR-NK cells, or MM1S cells plus frozen CAR-NK cells that were either treated pre-cryopreservation with Dasatinib for 24 hours or 72 hours or were not treated. (B) is a graphical quantification of the bioluminescence average radiance displayed in (8A).

FIGS. 9A-9E demonstrates that CAR NK cells cultured for 13 days and then treated with Dasatinib for 24 hours prior to freezing (MDACC) were superior to CAR NK cells stored with standard freezing media (CS10) protocols without deactivation. Additionally, the Dasatinib-treated cells demonstrated anti-tumor control in vivo that was similar to that of fresh CAR NK cells. (A) is a diagram illustrating the experimental procedure (animals were irradiated on day −4, injected with 0.5×10⁶ MM1S cells on day −3, injected with 5×10⁶ fresh CAR NK cells or 10×10⁶ frozen CAR NK cells on day 0, and imaged weekly). (B) Bioluminescence imaging (BLI) showing MM1S (CD70+ multiple myeloma cells, transduced with FireFlyluciferase (FFluc)) tumor growth over time (day −3, day 4, day 15, day 22, and day 29) in mice that received MM1S cells alone, MM1S cells plus Dasatinib, MM1S cells plus fresh (day 13 of culture) CD70-targeting CAR-NK cells, or MM1S cells plus frozen CAR-NK cells either treated pre-cryopreservation with Dasatinib for 24 hours or not treated pre-cryopreservation. (C) is a graphical quantification of the bioluminescence average radiance displayed in (9B). (D) is a graphical quantification of the absolute count of in vivo NK cell engraftment in the blood of mice 10 days or 20 days after receiving CAR-NK cells displayed in (9B). (E) displays the survival curves of the four groups of mice displayed in (9B), and (F) displays the associated statistical analysis.

FIGS. 10A-10D demonstrate CAR NK cells that were cultured for 13 days and treated with Dasatinib for 24 hours before undergoing freezing exhibited comparable in vivo antitumor control and engraftment capacities when compared to freshly prepared day 13 CAR NK cells. Furthermore, the data demonstrated that the activity of CAR NK cells remained unaffected when injected with freezing media. (A) is a diagram illustrating the experimental procedure (animals were irradiated on day-4, injected with 0.5×10⁶ MM1S cells on day −3, injected with 5×10⁶ fresh CAR NK cells or 10×10⁶ frozen CAR NK cells on day 0, and imaged weekly). (B) Bioluminescence imaging (BLI) showing MM1S (CD70+ multiple myeloma cells, transduced with FireFlyluciferase (Ffluc)) tumor growth over time (day −3, day 7, day 14, day 21, day 28 and day 35) in mice that received MM1S cells alone, MM1S cells plus fresh (day 13 of culture) CD70-targeting CAR-NK cells, or MM1S cells plus frozen CAR-NK cells. Frozen CAR NK cells were either resuspended in saline or freezing media in preparation for injection, and all CAR NK cells comprised the same constructs. (C) is a graphical quantification of the bioluminescence average radiance displayed in (10B). (D) is a graphical quantification of the percentage of in vivo NK cell engraftment in the blood of mice 10 days after receiving CAR-NK cells displayed in (10B).

FIGS. 11A-11C are FACS plots indicating that CAR NK cells that were cryopreserved after being cultured for 13 days and treated with Dasatinib for 24 hours prior to freezing exhibited in vivo engraftment levels that were comparable to those of freshly prepared (day 13 of culture) CAR NK cells at day 10 following infusion. Furthermore, the data demonstrated that the engraftment of CAR NK cells remained unaffected when injected with freezing media relative to saline. (A) Using negative and positive controls for CD138 (a tumor marker), and hCD45 (a natural killer cell marker), the quality of the antibodies and the gating strategy were evaluated. (B) Demonstrates the percentages of CD56/CD16+ and CD27+ (CAR marker) cells that were previously gated as CD138− and hCD45+ (left; example panel), fresh CD70-targeting CAR-NK cells (top right), and frozen CAR-NK cells either injected with saline (middle right) or freezing media (bottom right). (C) Demonstrates the percentages of hCD45+/CD138− (CAR NK cells) engraftment from 3 different tumor bearing mice that had been injected with either fresh (day 13 of culture) CD70-targeting CAR-NK cells or with pre-cryopreservation Dasatinib treated frozen CAR-NK cells. Frozen CAR-NK cells were thawed and either resuspended in saline or freezing media.

FIGS. 12A-12F depict the validation of the antitumor cytotoxicity of frozen CAR NK cells targeting TROP2 that were cultured for 13 days and treated with Dasatinib for 24 hours prior to freezing. The CAR NK cells treated with Dasatinib pre-cryopreservation demonstrated comparable antitumor cytotoxicity relative to fresh NK cells (day 13 of culture) in an in vivo mouse model of ovarian cancer (SKOV3 cells). (A) is a diagram of the experimental procedure performed. Animals were injected with 0.5×10⁶ SKOV3 cells on day −7; BLI began on day-2, animals were irradiated on day-1, and injected with 10×10⁶ fresh CAR NK cells or 10×10⁶ frozen CAR NK cells on day 0; and imaged weekly throughout life. (B) Bioluminescence imaging (BLI) showing SKOV3(TROP2+ ovarian cancer cells, transduced with FireFlyluciferase (FFluc)) tumor growth over time (day −2, day 5, day 12, day 19, day 26, day 33, day 40, day 47, day 54, day 61, and day 68) in mice that received no treatment, treatment with NT NK cells, treatment with fresh TROP2-targeting CAR NK cells, or treatment with pre-cryopreservation Dasatinib treated frozen/thawed TROP2-targeting CAR-NK cells (CAR NK cells comprised the same constructs). (C) is a graphical quantification of the bioluminescence average radiance displayed in (12B). (D) is a graphical quantification of the percentage of in vivo NK cell engraftment in the blood of mice 10 days or 20 days after receiving the NK cells displayed in (12B). (E) displays the survival curves of the four groups of mice in (12B), and (F) displays the associated statistical analysis.

FIGS. 13A-13B show how CAR NK cells treated with Dasatinib prior to freezing demonstrated comparable antitumor cytotoxicity to fresh NK cells in vivo in a mouse model of pancreatic ductal adenocarcinoma (PATC148). (A) displays bioluminescence imaging over time (day 3, day 6, day 13, day 20, day 27, day 34, and day 41) for the mice engrafted with PATC148 cells transduced with FireFlyluciferase (FFluc) with no treatment (PATC 148 alone), fresh NK cells transduced with a construct comprising TROP2-targeting CAR (iC9/TROP2CAR/IL15), or frozen CAR NK cells comprising the same construct that were treated with Dasatinib prior to freezing. (B) is a graphical quantification of the bioluminescence average radiance displayed in (13A).

FIGS. 14A-14C show that the addition of Dasatinib to NK cells prior to freezing enhanced their antitumor cytotoxicity post-thawing in in vitro glioblastoma tumor spheroid assays. (A) displays representative images of GSC272 spheroids (glioblastoma multiforme (GBM) tumor cell cancer stem cell lines transduced with mcherry) cultured alone (right column, top 2 panels), co-cultured with NK cells without Dasatinib treatment prior to freezing (column 1), or co-cultured with NK cells treated with Dasatinib prior to freezing (column 2), at E:T ratios of 1:1, 2:1, 3:1, or 5:1. Dasatinib alone was also added to some tumor cell comprising wells as control (right column, bottom 2 panels). (B) depicts quantification of total integrated red intensity over time showing a significant decrease in the total integrated red intensity (as a measure of tumor growth) when spheroids were co-cultured with NK cells that had been deactivated with Dasatinib prior to freezing. (C) depicts quantification of total integrated red intensity over time showing a significant decrease in the total integrated red intensity (as a measure of tumor growth) when GSC20 spheroids (GBM tumor cell cancer stem cell lines transduced with mcherry) were cultured alone, co-cultured with NK cells without Dasatinib treatment prior to freezing, or co-cultured with NK cells treated with Dasatinib prior to freezing. Together these results showed that NK cells deactivated with Dasatinib prior to freezing had in increased cytotoxicity against glioblastoma tumor cells when compared to NK cells that were not deactivated prior to freezing.

FIGS. 15A-15E shows the effects of various receptor tyrosine kinase inhibitors (Dasatinib, D; Bosutinib, B; Nilotinib, N; and Saracatinib, S; respectively) treatment before cryopreservation on NK cell viability and phenotypes after thawing. (A) depicts the recovery (survival) of NK cells following freeze/thawing where cells were treated with various tyrosine kinase inhibitors (Dasatinib, Bosutinib, Nilotinib, or Saracatinib; at final concentrations of 1 micromolar) pre-cryopreservation. Annexin V assay indicated the percentage of live anti-CD70 CAR NK cells (iCas9/CD27 CAR/IL-15) (Annexin V negative and live dead negative) after thawing to assess the recovery of NK cells treated with various kinase inhibitors before freezing. (B) depicts t-SNE analysis of thawed iC9/CD27 CAR/IL-15-NK cells (CAR) that were pre-treated with tyrosine kinase inhibitors (Dasatinib (D), Bosutinib (B), Nilotinib (N), or Saracatinib (S)) before cryopreservation; controls included untreated and cryopreserved CAR NK cells. Cells were evaluated post-thawing by CyTOF and merged to create a single t-SNE CUDA map (12,000 from 2 pooled donors per condition). FlowSOM analysis of the various conditions was then performed and the different FlowSOM metaclusters overlapped on the t-SNE CUDA map. Each colored region corresponded to a metacluster (1-10). (C) depicts a stacked bar graph with relative percentage frequencies of the different FlowSOM metaclusters for each of the CAR NK-cell conditions. (D) depicts contour plots showing the t-SNE CUDA cluster organization (as depicted in 15B) for the various CAR NK-cell conditions tested. (E) depicts a representative heatmap showing the expression levels of phenotypic and functional markers for the thawed CAR-NK cells. The Z-score for the expression level for each marker was represented by a color scale with bright red corresponding to the highest expression and dark blue to the lowest expression.

FIGS. 16A-16C depicts how addition of various tyrosine kinase inhibitors prior to freezing enhanced the antitumor cytotoxicity of CAR-NK cells post-thaw against solid cancer, as determined by xCELLigence impedance assays. The ovarian cancer cell line (SKOV3) (A) and renal cell carcinoma cell line (UMRC3) (B), were grown in 96 well RTCA E-Plates overnight. The next day, frozen iC9/CD27 CAR/IL-15-NK cells that were not pre-treated or were pre-treated with a tyrosine kinase inhibitor (Dasatinib, Bosutinib, Nilotinib, or Saracatinib) pre-cryopreservation were thawed and added at 2:1 effector to target (E:T) ratio. The cancer cell growth was measured continuously over time (X axis) by the xCELLigence device and represented as normalized cell index (Y axis). As shown in (C) compared to frozen/thawed iC9/CD27 CAR/IL-15-NK cells without any pre-treatment, frozen/thawed iC9/CD27 CAR/IL-15-NK cells that were pre-treated with tyrosine kinase inhibitors Dasatinib, Nilotinib, or Saracatinib, showed increased cytotoxicity against SKOV3 cells and/or UMRC3 cells.

FIGS. 17A-17C depicts how addition of various tyrosine kinase inhibitors prior to freezing enhanced the antitumor cytotoxicity of TCR-NK cells post-thaw against solid cancer, as determined by xCELLigence impedance assays. The melanoma cell line (A375) (A) and osteosarcoma cell line (Saos-2) (B), were grown in 96 well RTCA E-Plates overnight. The next day, frozen NYESO targeting TCR-NK cells that were not pre-treated or were pre-treated with a tyrosine kinase inhibitor (Dasatinib, Bosutinib, Nilotinib, or Saracatinib) pre-cryopreservation were thawed and added at 2:1 effector to target (E:T) ratio. The cancer cell growth was measured continuously over time (X axis) by the xCELLigence device and represented as normalized cell index (Y axis). As shown in (C) compared to frozen/thawed NYESO-TCR-NK cells without any pre-treatment, frozen/thawed NYESO-TCR-NK cells pre-treated with tyrosine kinase inhibitors Dasatinib, Nilotinib, Bosutinib, or Saracatinib showed increased cytotoxicity against A375 cells and/or Saos-2 cells.

FIGS. 18A-18B depicts how addition of Dasatinib pre-cryopreservation enhanced the antitumor cytotoxicity of NYESO targeting TCR-NK cells post-thaw against multiple myeloma cells as shown by chromium release assays. (A) Multiple myeloma cells (U266) were labelled with chromium-51 and co-cultured with frozen and thawed NYESO-TCR-NK cells that were not treated pre-cryopreservation or were treated pre-cryopreservation with various tyrosine kinase inhibitors (Dasatinib, Bosutinib, Nilotinib, or Saracatinib) at various E:T ratios (2:1, 1:1, 0.5:1, or 0.25:1). After four hours, chromium release was measured, which corresponded to cancer cell death. (B) Compared to NYESO-TCR-NK cells that were frozen without any pre-treatment, frozen NYESO-TCR-NK cells pre-treated with Dasatinib showed increased cytotoxicity against U266 cells.

### DETAILED DESCRIPTION

One limitation of using certain cryopreserved cells for clinical therapy is their small numbers and poor survival rates post thaw. In some embodiments, the present disclosure has addressed both of these limitations by using a GMP-compliant strategy for the ex vivo activation and/or expansion of cells, and subsequent temporary deactivation of said cells prior to cryopreservation, wherein such deactivation prior to cryopreservation is performed by treatment with at least one deactivating agent and results in effector cells that can be stored at sufficiently high numbers. Post thaw, the cells have improved survival rates, improved transgene expression rates, and/or improved cytotoxicity relative to their non-deactivated cryopreserved and subsequently thawed counterparts. In specific embodiments, any method disclosed herein indicates that this strategy could also be applied to cells without prior expansion and/or activation.

Accordingly, certain embodiments of the present disclosure provide methods and compositions concerning the preservation, such as for storage, of clinical-grade cells, including those intended for immunotherapy comprising effector cells. Growing and molding clinically relevant numbers of cells for infusion into patients while meeting time constraints is an extremely challenging endeavor even in the best of circumstances. The disclosed methods and compositions detail the technical processes of cellular preservation suitable for improving cell viability, cytotoxicity, and/or transgene expression levels relative to their control counterparts that do not comprise preparation using methodologies described herein.

In particular embodiments, further provided herein is a method for deactivating any type of mammalian cell. In some embodiments, the mammalian cells may be of any kind, including immune cells of any kind, including NK cells, T cells, B cells, NKT cells, macrophages and monocytes, gamma delta T cells, regulatory T cells, stem cells, induced pluripotent stem cells (iPSCs) or any cell derived from iPSCs, MSCs, hematopoietic stem cells, differentiated or committed cells from any organ, any fibroblasts. In particular embodiments, provided herein are methods for deactivating immune cells such as effector cells. In any case, the mammalian cells may be utilized for adoptive cell therapy. In some embodiments, the cells are NK cells. In some embodiments, the cells are not T cells. In some embodiments, the cells are NK cells comprising one or more transgenes. In some embodiments, a transgene is a chimeric antigen receptor (CAR), engineered cytokine and/or cytokine signaling pathway member, and/or a T cell receptor (TCR). In some embodiments, the NK cells may be modified by the hand of man in one or more ways, such as but not limited to, introduction of one or more engineered antigen receptors, including chimeric antigen receptors or T cell receptors, or CD16, CD32 and/or CD64 receptor. In some embodiments, the NK cells may express a heterologous cytokine, such as IL-2, IL-4, IL-7, IL-12, IL-15, IL-18, IL-21 and/or IL-23. In some embodiments, the NK cells may express a suicide gene. In a specific embodiment, the NK cells comprise a chimeric antigen receptor that targets a tumor antigen and a cytokine, such as IL-15, optionally with a suicide gene. In some embodiments, NK cells are gene edited using any methods. In some embodiments NK cells are combined with a monospecific, bispecific, and/or multi-specific antibody either ex vivo or in vivo. In some embodiments NK cells are activated and/or expanded or used directly ex vivo without prior activation/expansion.

In specific embodiments, following cell deactivation cells may be placed in a suitable cryopreservation media (e.g., freezing media). In some embodiments, a freezing media may comprise a cryoprotectant such as (but not limited to) dimethyl sulfoxide (DMSO), glycerin, glycerol, hydroxyethyl starch, or a combination thereof, serum from human, bovine or other animal source, or a serum alternative such as (but not limited) to platelet lysate, one or more cytokines or growth factors included but not limited to IL-1, IL-2, IL-3, IL-4, IL-6, IL-7, IL-9, IL-10, IL-12, IL-13, IL-15, IL-17, IL-18, IL-21, IL-22, interferon, tumor necrosis factor, stem cell factor, FLT3-ligand, APRIL, or a combination thereof. Serum may be utilized as a source of growth factors, adhesion factors, hormones, lipids and/or minerals and/or in certain cases is used to regulate cell membrane permeability and serves as a carrier for lipids, enzymes, micronutrients, and trace elements into the cell. In some embodiments, the freezing media allows for improved rates of successful freezing of individual doses of cells with improved viability and functionality. In some embodiments, the cells may be thawed and infused into patients per demand. Thus, the deactivated and frozen cells provide herein are an “off-the-shelf” cell therapy that can be thawed and infused into patients with no delay needed for production.

The methods and compositions described herein provide for adoptive cell therapy methods and cells to be stored as banks of cells for any purpose, including immunotherapy, without the need to recruit donors for cell collection, although this approach may also be used for the cryopreservation of autologous patient-directed products, as well.

### I. Definitions

It is contemplated that any embodiment discussed in this specification can be implemented with respect to any method or composition of the invention, and vice versa. Furthermore, compositions of the invention can be used to achieve methods of the invention.

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

Throughout this application, the term “about” is used to indicate that a value includes the inherent variation of error for the device, the method being employed to determine the value, or the variation that exists among the study subjects.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

The term “deactivate” or “deactivated” as used herein refers to a process or refers to a cell that has been induced into a less active state through exposure to one or more agents. A deactivated cell may also be considered at “rest” or a “resting” cell. In general as described herein, deactivation is a temporary process that can be lifted upon removal of the one or more agents that induced the deactivated state. In general, a deactivated cell will have a reduction or modified level of cytolytic activity, cytokine production, proliferation of NK cells, and/or one or more cytotoxicity markers such as but not limited to CD95, NKG2D, DNAM, OX-40, TRAIL, HLA-DR, CD2, CD25, ICOS, CCR5, CD62L, CXCR4, and/or C-kit relative to a cell that has not been deactivated.

The term “engineered” as used herein refers to an entity that is generated by the hand of man, including a cell, nucleic acid, polypeptide, vector, and so forth. In at least some cases, an engineered entity is synthetic and comprises elements that are not naturally present or configured in the manner in which it is utilized in the disclosure. In specific embodiments, a vector is engineered through recombinant nucleic acid technologies, and a cell is engineered through transfection or transduction of an engineered vector.

An “immune disorder,” “immune-related disorder,” or “immune-mediated disorder” refers to a disorder in which the immune response plays a key role in the development or progression of the disease. Immune-mediated disorders include autoimmune disorders, allograft rejection, graft versus host disease and inflammatory and allergic conditions.

An “immune response” is a response of a cell of the immune system, such as a B cell, or a T cell, or innate immune cell to a stimulus. In one embodiment, the response is specific for a particular antigen (an “antigen-specific response”).

An “autoimmune disease” refers to a disease in which the immune system produces an immune response (for example, a B-cell or a T-cell response) against an antigen that is part of the normal host (that is, an autoantigen), with consequent injury to tissues. An autoantigen may be derived from a host cell, or may be derived from a commensal organism such as the micro-organisms (known as commensal organisms) that normally colonize mucosal surfaces.

The term “load,” “loaded,” or loading” as used herein refers to adoptive cell therapy cells having one or more antibodies bound to the cells on the surface of the cells.

The term “pre-load,” “pre-loaded,” or “pre-loading” as used herein refers to adoptive cell therapy cells that have had one or more antibodies bound to the cells on the surface of the cells prior to use of the cells for any reason.

The phrases “pharmaceutical or pharmacologically acceptable” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure. Moreover, for animal (e.g., human) administration, it will be understood that preparations should meet sterility, pyrogenicity, general safety, and purity standards as required by FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

The term “pre-activated” or “pre-activation” as used herein refers to exposure of NK cells to IL-12, IL-15 or IL-2, and/or IL-18 that results in increased signaling pathways related to NK cell effector function, such as enrichment of genes involved in the IFN-γ response, TNF signaling, IL-2/STAT5 signaling, IL-6/JAK/STAT3 signaling, mTOR pathway and/or genes related to inflammatory immune responses. In specific cases there is increased expression of TRAIL, NKp44 and/or CD69.

As used herein, “prevent,” and similar words such as “prevented,” “preventing” etc., indicate an approach for preventing, inhibiting, or reducing the likelihood of the occurrence or recurrence of, a disease or condition, e.g., cancer. It also refers to delaying the onset or recurrence of a disease or condition or delaying the occurrence or recurrence of the symptoms of a disease or condition. As used herein, “prevention” and similar words also includes reducing the intensity, effect, symptoms and/or burden of a disease or condition prior to onset or recurrence of the disease or condition.

“Subject” and “patient” and “individual” may be interchangeable and may refer to either a human or non-human, such as primates, mammals, and vertebrates. In particular embodiments, the subject is a human. The subject can be any organism or animal subject that is an object of a method or material, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals. The subject can be a patient, e.g., have or be suspected of having a disease (that may be referred to as a medical condition), such as one or more infectious diseases, one or more genetic disorders, one or more cancers, or any combination thereof. The “subject” or “individual”, as used herein, may or may not be housed in a medical facility and may be treated as an outpatient of a medical facility. The individual may be receiving one or more medical compositions via the internet. An individual may comprise any age of a human or non-human animal and therefore includes both adult and juveniles (e.g., children) and infants and includes in utero individuals. A subject may or may not have a need for medical treatment; an individual may voluntarily or involuntarily be part of experimentation whether clinical or in support of basic science studies.

“Treating” or treatment of a disease or condition refers to executing a protocol, which may include administering one or more drugs or cellular therapy products to a patient, in an effort to alleviate signs or symptoms of the disease. Desirable effects of treatment include decreasing the rate of disease progression, ameliorating or palliating the disease state, and remission or improved prognosis. Alleviation can occur prior to signs or symptoms of the disease or condition appearing, as well as after their appearance. Thus, “treating” or “treatment” may include “preventing” or “prevention” of disease or undesirable condition. In addition, “treating” or “treatment” does not require complete alleviation of signs or symptoms, does not require a cure, and specifically includes protocols that have only a marginal effect on the patient.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, complete eradication of the tumor, a reduction in the size of a tumor, a reduction in the invasiveness of a tumor, reduction in the growth rate of the cancer, or prevention of metastasis. Treatment of cancer may also refer to prolonging survival of a subject with cancer.

The term “antigen presenting cells (APCs)” refers to a class of cells capable of presenting one or more antigens in the form of a peptide-MHC complex recognizable by specific effector cells of the immune system, and thereby inducing an effective cellular immune response against the antigen or antigens being presented. The term “APC” encompasses intact whole cells such as macrophages, B-cells, endothelial cells, activated T-cells, dendritic cells, cell lines (such as K562), or molecules, naturally occurring or synthetic, capable of presenting antigen, such as purified MHC Class I molecules complexed to 02-microglobulin.

### II. Deactivation and Cryopreservation of Cells

Cells of any kind may be deactivated and subsequently cryopreserved as described herein. The cells may be mammalian, in certain embodiments, and in specific cases they are mammalian cells to be utilized for research and/or therapy. The cells may be immune cells, in specific cases, including immune cells to be utilized for adoptive cell therapy. Such cells may or may not be NK cells, T cells, NKT cells, B cells, macrophages or monocytes, stem cells, induced pluripotent stem cells (iPSCs) or any cell derived from iPSCs, MSCs, hematopoietic stem cells, differentiated or committed cells from any organ, any fibroblasts, and so forth. In certain embodiments, the cells are NK cells. The cells may be obtained from an individual, cryopreserved using media encompassed herein, and then thawed and used for the individual and/or for another one or more other individuals. The cells may be obtained from an individual, manipulated to comprise one or more characteristics, deactivated and cryopreserved as described herein, and used for the individual and/or for another one or more other individuals.

A first plurality of cells from one collection of cells may be deactivated and cryopreserved using one or more particular deactivating agents and one or more particular cryopreservation media described in the art, while optionally a second plurality of cells from the same collection of cells may be deactivated and cryopreserved using one or more different deactivating agents and different cryopreservation media. Such a practice may or may not be employed depending on the application of the cells, the number and/or viability of the cells, and so forth. Exemplary cryopreservation media solutions are described in the international publication WO2021041399 “Cell cryopreservation medium” which is incorporated herein by reference for the purposes described herein.

In particular embodiments, cells are deactivated and cryopreserved as described herein substantially immediately following collection (e.g., isolation) of them from one or more individuals or from one or more sources (e.g., cryopreserved blood banks). In some embodiments, cells deactivated and cryopreserved as described herein are deactivated and cryopreserved after culture and/or expansion. In some embodiments, cells are pre-activated and/or activated as described herein prior to deactivation and cryopreservation. Following expansion and/or activation, the cells (such as immune cells) may be immediately manipulated for a later purpose (such as infusion), or they may be deactivated and stored (e.g., through cryopreservation). In certain aspects, the cells may be propagated for days, weeks, or months ex vivo as a bulk population within about 1, 2, 3, 4, 5, 6, 7, 8, 9, 10, 11, 12, 13, 14, or more days following cell collection (e.g., cell isolation).

In particular embodiments, a complete media change may need to be performed for cells prior to deactivation and/or cryopreservation. In some embodiments, a complete media change may need to be performed for cells based on the viable cell concentration prior to deactivation and/or cryopreservation. In some embodiments, a complete media change may need to be performed for cells prior to deactivation and/or cryopreservation, if the viable cell concentration is at least about 1×10⁴, 1×10⁵, 1×10⁶, 1×10⁷, or 1×10⁸ cells/mL. In some embodiments, a complete media change may need to be performed for cells prior to deactivation and/or cryopreservation, if the viable cell concentration is at least about 1×10⁶ cells/mL. In some embodiments, the cell culture media may be changed with warm complete media. In some embodiments, the warm complete media comprises any one of, any combination of, or all of RPMI, Clicks, and Human AB serum. In some embodiments, the warm complete media may be warmed to 37° C. for at least about 5 minutes, 10 minutes, 15 minutes, 20 minutes, or 25 minutes before the media change. In some embodiments, the warm complete media may be warmed to 37° C. for at least about 15 minutes before the media change.

In particular embodiments, a complete media change may not need to be performed for cells prior to deactivation and/or cryopreservation. In some embodiments, a complete media change may not need to be performed for cells prior to deactivation and/or cryopreservation, if the viable cell concentration equals or is less than about 1×10⁴, 1×10⁵, 1×10⁶, 1×10⁷, or 1×10⁸ cells/mL. In some embodiments, a complete media change may not need to be performed for cells prior to deactivation and/or cryopreservation, if the viable cell concentration equals or is less than about 1×10⁶/mL.

In particular embodiments, a cell for deactivation and/or cryopreservation is an NK cell. In certain embodiments, an NK cell surface antigen may be CD16, CS1, CD56, NKG2D, NKG2C, or any c-type lectin, a costimulatory molecule such as DNAM, 2B4, CD2, an NCR, or KIR. In some embodiments, the source of the NK cells is from cord blood (CB). In some embodiments, the source of the cord blood is cord blood from 1 donor or pooled from 2 or more individual cord blood units. The CB may be pooled from 3, 4, 5, 6, 7, or 8 individual cord blood units. In some embodiments, the NK cells (which may be CD56+) may be derived from cord blood mononuclear cells, from cord blood, from bone marrow or peripheral blood hematopoietic stem cells, from iPSCs, from peripheral blood, or from NK cell lines. In some embodiments, a source of the NK cells may be a fresh source or cryopreserved repository. In some cases, when the NK cells are sourced from cryopreservation, the NK cells were cryopreserved in a medium comprising at least one cryoprotectant, at least one serum or non-serum alternative to serum.

In certain embodiments, a deactivating agent may have a pharmacological effect on the activity of a cell, see for example Mestermann K, Giavridis T, Weber J, et al. The tyrosine kinase inhibitor Dasatinib acts as a pharmacologic on/off switch for CAR T cells. Sci Transl Med. 2019; 11(499); and/or Weber E W, Lynn R C, Sotillo E, Lattin J, Xu P, Mackall CL. Pharmacologic control of CAR-T cell function using Dasatinib. Blood Adv. 2019; 3(5):711-717; each of which are incorporated herein by reference for the purposes described herein.

In certain embodiments, a deactivating agent(s) is washed from the cells one or more times prior to cryopreservation.

### A. NK Cells

In some embodiments, cells are immune cells. In certain embodiments, cells are NK cells. In certain embodiments, cells are non-transduced NK cells (e.g., NK cells without any transduction of a construct). In certain embodiments, cells are transduced NK cells (e.g., NK cells transduced with a construct encoding a functional molecule, e.g., a CAR, TCR, cytokine, etc.). NK cells are emerging as an exciting source of cellular immunotherapy for patients with malignant hematologic disease as well as solid tumors; however, most studies using non-fresh adoptively transferred NK cells have been limited by inadequate persistence, poor in vivo expansion and disappointing anti-tumor activity of the infused cells. Thus, a barrier to overcome in the field of NK immunotherapy is the need for biology-driven approaches to increase NK cell availability while maintaining and/or improving antitumor functionality, such as by manipulation of the cells prior to administration as therapy. Accordingly, in certain embodiments, the present disclosure provides methods for expansion and/or activation, deactivation, and optionally storage of NK cells having enhanced efficacy of any kind compared to NK cells that are not so manipulated. Some embodiments of the present disclosure concern the isolation, activation, expansion, deactivation, cryopreservation, thawing, and use of NK cells, including for cancer immunotherapy.

In particular embodiments, the disclosure encompasses loaded (optionally), pre-activated (optionally), and expanded (optionally) NK cells that are then deactivated as described herein, and stored (optionally). In some embodiments, such cells exhibit enhanced anti-tumor functionality against cancer. In some embodiments, such cells display enhanced antibody-dependent cellular cytotoxicity (ADCC). In some embodiments, such cells exhibit enhanced transgene expression. In some embodiments, such cells exhibit enhanced survival rates. In some embodiments, such cells may have decreased expression of one or more genes.

In certain embodiments, NK cells described herein may be derived from any suitable source, such as cord blood (CB), including human CB. In particular cases, the NK cells are not derived from cord tissue (the insulating material (i.e., the Wharton's jelly) surrounding the vessels of the umbilical cord). In alternative embodiments, the NK cells are derived from human peripheral blood mononuclear cells (PBMC), unstimulated leukapheresis products (PBSC), human embryonic stem cells (hESCs), induced pluripotent stem cells (iPSCs), and/or bone marrow, or cord blood, bone marrow or peripheral blood hematopoietic stem cells, or NK cells lines derived from patients, such as NK-92, by methods well known in the art. In particular embodiments, the NK cells are isolated from pooled CB. The CB may be pooled from 2, 3, 4, 5, 6, 7, 8, 9, 10, or more units. The NK cells may be autologous or allogeneic with respect to a recipient individual. The isolated NK cells may or may not be haplotype matched for the subject to be administered the cell therapy. NK cells may or may not be detected by specific surface markers, such as CD16 and/or CD56 in humans. In some cases, the NK cells are depleted for the presence of one or more surface markers, such as depleted for CD3+, CD14+ and/or CD19+ cells. In specific embodiments, the NK cells are CD3− CD56+.

In certain aspects, the NK cells are isolated by the previously described method of ex vivo expansion of NK cells (Spanholtz et al., 2011; Shah et al., 2013). In this method, CB mononuclear cells are isolated by ficoll density gradient centrifugation. The cell culture may be depleted of any cells expressing CD3 and may be characterized to determine the percentage of CD56+/CD3−cells or NK cells. In other methods, umbilical CB is used to derive NK cells by the isolation of CD34+ cells.

### B. Activating Agents

In some embodiments, cells are expanded and/or activated prior to deactivation. Expansion and/or activation of cells can be through any suitable means known in the art.

In particular embodiments, NK cells are expanded in a particular manner and optionally are pre-activated in a particular manner. For example, the NK cells may be expanded in the presence of particular antigen presenting cells under particular culture conditions, in addition to the NK cells optionally being exposed to one or more cytokines as a pre-activation step. In some embodiments, following cell isolation, cells are pre-activated using a cytokine cocktail. In some embodiments, a cytokine cocktail comprises one or more, or any combination of, IL-2, IL-12, IL-15, and/or IL-18.

In some embodiments, the NK cells are pre-activated prior to optional expansion and/or additional activation, and deactivation and/or storage. The pre-activation step may or may not occur before any expansion step. In specific embodiments, the NK cells are pre-activated with one or more cytokines, and in specific embodiments, the NK cells are pre-activated with one or more of IL-12, IL-15, IL-2, and IL-18 and including two, three, or more. In cases where less than all three of IL-12, IL-15, and IL-18 are utilized, it may be that IL-12 and IL-15 but not IL-18; or IL-12 and IL-18 but not IL-15; or IL-15 and IL-18 but not IL-12. IL-2 may or may not be substituted for IL-15.

In some embodiments, the pre-activation cytokines are IL-12, IL-15, and IL-18. One or more additional cytokines may be used for the pre-activation step. The pre-activation may be for a short period of time such as 5-72 hours, such as 10-50 hours, particularly 10-20 hours, such as 12, 13, 14, 15, 16, 17, 18, 19, or 20 hours, and specifically about 16 hours in some cases. In some embodiments, the pre-activation culture may comprise IL-18 and/or IL-15 at a concentration of 10-100 ng/mL, such as 40-60 ng/mL, particular 45, 46, 47, 48, 49, 50, 51, 52, 53, 54, or 55 ng/mL, specifically about 50 ng/mL. In some cases, the pre-activation culture comprises IL-12 at a concentration of 0.1-150 ng/mL, including at a concentration of 1-20 ng/mL, such as a concentration of 10 ng/mL. In alternative embodiments the NK cells may be stimulated with IL-2, or other cytokines that bind the common gamma-chain (e.g., IL-7, IL-21, and others), and this may be in addition to IL-12, IL-15, and IL-18 or as an alternative to one or more of them. In such cases, the pre-activation culture may comprise IL-12 at a concentration of 0.1-150 ng/mL, such as 0.5-50 ng/mL, particularly 1-20 ng/mL, such as 2, 3, 4, 5, 6, 7, 8, 9, 10, 11, 12, 13, 14, or 15 ng/mL, specifically about 10 ng/mL.

In particular embodiments, following an optional pre-activation, NK cells are expanded to increase their quantity prior to deactivation and/or storage. The expanded cells may or may not be derived from pre-activated NK cells such that a pre-activation step may occur before an expansion step. The NK cell expansion step may be of any suitable length such that the NK cell population is expanded, but in specific cases the expansion step utilizes particular one or more reagents, such as in culture, to enhance their expansion. In certain cases the NK cells may not be expanded. IL-2 or IL-15 or IL-18 or any combination of the cytokines may be added to the expansion culture before or during expansion. In some embodiments, the NK cells can be expanded ex vivo in flasks. In some embodiments, the NK cells can be expanded ex vivo in several different bioreactor configurations with continuous perfusion of media/additives.

In some embodiments, the NK cells (whether pre-activated or not) may be washed (e.g., with PBS or Plasma Lyte or human serum albumin or culture media or combinations thereof) prior to and/or after expansion, such as 2, 3, 4, or 5 times, specifically 3 times. In particular embodiments, the NK cells are expanded in the presence of a feeder cell and/or an artificial antigen presenting cell (APC) such as an universal artificial antigen presenting cells (uAPCs) and/or artificial antigen presenting cells (aAPCs). The aAPCs may be engineered to express CD137 ligand and/or a membrane-bound cytokine. The membrane-bound cytokine may be membrane-bound IL-21 (mIL-21) or membrane-bound IL-15 (mIL-15). In particular embodiments, the aAPCs are engineered to express CD137 ligand and mIL-21. The aAPCs may be derived from cancer cells, such as leukemia cells. The aAPCs may not express endogenous HLA class I, II, or CD1d molecules. They may express ICAM-1 (CD54) and LFA-3 (CD58) or CD48. In particular, the aAPCs may be K562 cells, such as K562 cells engineered to express CD137 ligand and mIL-21. The engineering may be by any method known in the art, such as retroviral transduction, although any viral or non-viral vector may be utilized. The aAPCs may or may not be irradiated.

In some embodiments, antigen-presenting cells may be macrophages, B lymphocytes, and/or dendritic cells, which are distinguished by their expression of a particular MHC molecule. APCs internalize antigen and re-express a part of that antigen, together with the MHC molecule on their outer cell membrane. The MHC is a large genetic complex with multiple loci. The MHC loci encode two major classes of MHC membrane molecules, referred to as class I and class II MHCs. T helper lymphocytes generally recognize antigen associated with MHC class II molecules, and T cytotoxic lymphocytes recognize antigen associated with MHC class I molecules. In humans the MHC is referred to as the HLA complex and in mice the H-2 complex.

In some cases, aAPCs are useful in preparing therapeutic compositions and cell therapy products of the embodiments. For general guidance regarding the preparation and use of antigen-presenting systems, see, e.g., U.S. Pat. Nos. 6,225,042, 6,355,479, 6,362,001 and 6,790,662; U.S. Patent Application Publication Nos. 2009/0017000 and 2009/0004142; and International Publication No. WO2007/103009.

In some embodiments, APC systems (e.g., uAPC and/or aAPC) may comprise at least one exogenous assisting molecule. Any suitable number and combination of assisting molecules may be employed. The assisting molecule may be selected from assisting molecules such as co-stimulatory molecules and adhesion molecules. Exemplary co-stimulatory molecules include CD86, CD64 (FcγRI), 41BB ligand, and IL-21. Adhesion molecules may include carbohydrate-binding glycoproteins such as selectins, transmembrane binding glycoproteins such as integrins, calcium-dependent proteins such as cadherins, and single-pass transmembrane immunoglobulin (Ig) superfamily proteins, such as intercellular adhesion molecules (ICAMs), which promote, for example, cell-to-cell or cell-to-matrix contact. Exemplary adhesion molecules include LFA-3 and ICAMs, such as ICAM-1. Techniques, methods, and reagents useful for selection, cloning, preparation, and expression of exemplary assisting molecules, including co-stimulatory molecules and adhesion molecules, are exemplified in, e.g., U.S. Pat. Nos. 6,225,042, 6,355,479, and 6,362,001.

In some embodiments, the expansion may be for a particular duration in time, such as for about 2-30 days, such as 3-20 days, particularly 12-16 days, such as 12, 13, 14, 15, 16, 17, 18, or 19 days, specifically about 14 days. In some embodiments, the optionally pre-activated NK cells and APCs may be present at a ratio of about 3:1-1:3, such as 2:1, 1:1, 1:2, specifically about 1:2. In some embodiments, the expansion culture may further comprise one or more cytokines to promote expansion, such as IL-2. The IL-2 may be present at a concentration of about 10-500 U/mL, such as 100-300 U/mL, particularly about 200 U/mL. The IL-2 may be replenished in the expansion culture, including at a certain frequency, such as every 2-3 days. In some embodiments, the APCs may be added to the culture at least a second time, such as at about 7 days of expansion. Any cytokine(s) used in the pre-activation and/or expansion steps may be recombinant human cytokines.

In some embodiments, following expansion, the NK cells may be immediately deactivated, such as through treatment with one or more deactivating agent as described herein. Following deactivation, NK cells may be stored, such as by cryopreservation. In certain aspects, the cells may be propagated for days, weeks, or months ex vivo as a bulk population within about 1, 2, 3, 4, or 5 days following cell isolation.

In certain embodiments, activated and/or expanded NK cells can secrete type I cytokines, such as interferon-γ, tumor necrosis factor-α and granulocyte-macrophage colony-stimulating factor (GM-CSF), which activate both innate and adaptive immune cells as well as other cytokines and chemokines. In some embodiments, the measurement of these cytokines can be used to determine the activation status of NK cells. In addition, other methods known in the art for determination of NK cell activation may be used for characterization of the NK cells of the present disclosure. In some embodiments, measurement of such markers can be utilized to determine the efficacy of one or more deactivating agents, and/or the status of the NK cells activation phenotypes.

In some embodiments, with respect to particular pre-activation and expansion aspects of the disclosure, in specific embodiments the NK cells pre-activated with IL-12, IL-15, and IL-18 followed by expansion with APCs. In some embodiments, an APC is an aAPC, such as K562 cells expressing mIL-21 and CD137 ligand, provide a highly potent cellular product. In some embodiments, provided herein are NK cells for the treatment of various diseases, such as immunotherapy of patients with cancer, that are suitable for long-term storage such as through cryopreservation. In an exemplary method, an isolated NK cells may be subjected to a brief period, such as about 16 hours, of pre-activation with a combination of cytokines, such as interleukin-12 (IL-12), IL-15, and/or IL-18, followed by expansion using APCs, and/or exogenous IL-2, IL-2 or IL-15 or IL-18 or any combination of the cytokines may be added to the expansion culture at least a second time, following a suitable period of expansion, NK cells are deactivated by treatment with a deactivating agent for a suitable period of time, following deactivation NK cells can be stored such as through cryopreservation, following cryopreservation NK cells can be thawed and then utilized for any suitable downstream process, such as but not limited to, immunotherapy and/or additional manipulation (e.g., transformation, transduction, loading, etc.).

### C. Loading of NK Cells

In some embodiments, cells are optionally loaded with an agent, such as an antibody.

In certain embodiments, the NK cells may be loaded in any specific manner, including in culture, immediately before infusion, and/or added in vivo, for example, to produce a complex of NK cells with the antibodies. The conditions are suitable enough to allow for an effective amount of antibody to bind to the surface of the NK cells. In the case of use of monospecific antibodies, the Fc region of the monospecific antibody binds the NK cell while the antigen binding domain of the monospecific antibody is free to bind its target antigen. In the case of use of multispecific antibodies, one or more antigen binding domains of the antibody may bind to the surface of the NK cells, such as through an antigen on the surface of the NK cells, and the other antigen binding domain is free to bind its target antigen. In alternative cases of use of multispecific antibodies, one or more antigen binding domains of the antibody may bind to one target antigen on a target cell, and one or more alternative antigen binding domains of the antibody may bind to one or more alternative target antigens on a target cell.

In some embodiments, the culture conditions by which the NK cells become loaded may or may not be of a particular type having one or more specific parameters. In particular embodiments, the loading of the NK cells occurs in culture at a specific temperature, such as 37° C., although in alternative embodiments the temperature is 36° C. or 38° C., or lower or higher. In some embodiments, the duration of the loading step may be for any suitable amount of time, such as in a range of one minute to 24 hours or longer. For example, the range may be in the range of 1 min to 24 hrs, 1 min to 18 hrs, 1 min to 12 hours, 1 min to 6 hrs, 1 min to 1 hr, 30 min to 24 hrs, 30 min to 18 hrs, 30 min to 12 hrs, 30 min to 6 hrs, 30 min to 1 hr, 1-24 hrs, 1-18 hrs, 1-12 hrs, 1-6 hrs, 6-24 hrs, 6-18 hrs, 6-12 hrs, 12-24 hrs, 12-18 hrs, or 18-24 hrs. In some embodiments, the duration of the loading step may be greater than or equal to approximately 6, 8, 10, 12, 14, 16, 18, 20, 22, 24, 26, 28, 30, 32, 34, 36, 38, 40, 42, 44, 46, or 48 hours, or any range derivable therein. In specific embodiments, the cell culture media is basal media or complex media. In some cases, the culture comprises one or more reagents that were utilized during pre-activation and/or expansion steps, while in other cases the culture does not. In specific embodiments, the culture comprises one or more cytokines, including one or more of IL-12, IL-15, IL-2, and IL-18, for example. In some embodiments, the culture comprises APCs of any kind.

In some embodiments, antibodies are subjected in an effective amount to an effective amount of NK cells of the disclosure, thereby producing a complex that is “chimeric antigen receptor-like.” In some embodiments, an antigen binding domain of the antibody binds to the NK cells, such as through the antigen that is a cell surface protein. In some embodiments, a plurality of antibodies may be subjected to a plurality of NK cells such that there are multiple complexes of cell/antibody. In some embodiments, antibodies may be of any type, including monospecific, bispecific, or multispecific, and in specific cases the antibody engages both the NK cell and a target antigen through an antigen binding domain of the antibody (such as with engagers in the art that are fusion proteins consisting of two single-chain variable fragments (scFvs) of different antibodies). In some embodiments, wherein the antibody is monospecific, an antigen binding domain of the antibody binds a target antigen, such as a cancer antigen, and another part of the antibody binds the NK cells, such as an Fc region of the antibody. In some embodiments, wherein the antibody is multispecific, one or more antigen binding domains of the antibody binds the NK cell (such as through an NK cell surface antigen) and one or more antigen binding domains of the antibody binds one or more target antigens. In some embodiments, a multispecific antibody may be bispecific, trispecific, or tetraspecific, for example. In some embodiments, wherein the antibody is trispecific or tetraspecific, the additional antigen binding domains may bind other cells, such as stem cells.

In some embodiments, the antibodies may bind any NK cell surface antigen (that may or may not be receptors) on NK cells, such as CD16 (including CD16a or CD16b), CD56, a c-type lectin such as NKG2D, NKG2C, a costimulatory molecule such as CS1, DNAM, 2B4, CD2, an NCR, or KIR, and redirect the NK cells to a target, thus increasing the response and specificity against different tumors.

In some embodiments, the antibodies may bind any suitable antigen (e.g., antigens described herein, such as those that are described as targets of CARs and/or TCRs). In particular embodiments, an antibody targets EGFR. In particular embodiments, an antibody is bi-specific and targets EGFR and c-MET.

In some embodiments, generation of the complexes may be by any suitable means, such that the conditions are sufficient for the appropriate region of the antibody to bind the appropriate surface region of the NK cell. In some embodiments, any particular medium may be utilized. In specific cases, Plasma-Lyte A and/or human serum albumin are utilized, wherein in other cases they are not. Once the complexes are formed in culture, they may or may not be washed prior to deactivation or storage.

### D. Deactivating Agents

In some embodiments, cells are deactivated by treating the cells with a deactivating agent. In some embodiments, cells are deactivated by treating the cells with a deactivating agent prior to storage, for example but not limited to, cryopreservation, and under conditions suitable for the cells to become deactivated.

In some embodiments, a deactivating agent comprises one or more kinase inhibitors. In some embodiments, a deactivating agent is a tyrosine kinase (TK) inhibitor. In some embodiments, a cell is an NK cell and it is treated with a deactivating agent under conditions to produce a deactivated NK cell. In some embodiments, an NK cell is treated with a TK inhibitor to produce a deactivated NK cell. In some embodiments, an NK cell is treated with Dasatinib to produce a deactivated NK cell. In some embodiments, an NK cell is treated with Dasatinib, Nilotinib, Imatinib, Bosutinib, Saracatinib, and/or an mTOR inhibitor (e.g., rapamycin etc.) to produce a deactivated NK cell. In some embodiments, an NK cell is not treated with Bosutinib. In some embodiments, an NK cell is not treated with nilotinib. In some embodiments, an NK cell is not treated with Saracatinib. In some embodiments, an NK cell is not treated with an mTOR inhibitor.

In some embodiments, a deactivating agent is an agent that can be removed following a suitable treatment period. In some embodiments, the deactivating effects of a deactivating agent are reversable by removal of the deactivating agent. In some embodiments, following removal of a deactivating agent, cells (e.g., NK cells) can spontaneously reactivate without the addition of additional activating agents. In some embodiments, a deactivating agent is removed by washing off of the deactivating agent, and one of skill in the art understands the steps required to undertake cell culture, such as the expansion of cells, splitting of cells, washing of cells, etc. In some embodiments, a deactivating agent is removed prior to storage (e.g., cryopreservation) of a cell (e.g., an NK cell). In some embodiments, a deactivating agent is removed following storage (e.g., cryopreservation) of a cell (e.g., an NK cell).

In some embodiments, a cell (e.g., an NK cell) is treated with a deactivating agent for more than, less than, or exactly about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 hours, or any range derivable therein. In certain embodiments, an NK cell is treated with a deactivating agent for more than, less than, or exactly about 16 hours. In certain embodiments, an NK cell is treated with a deactivating agent for more than, less than, or exactly about 24 hours. In certain embodiments, an NK cell is treated with a deactivating agent for more than, less than, or exactly about 36 hours. In certain embodiments, an NK cell is treated with a deactivating agent for more than, less than, or exactly about 48 hours. In certain embodiments, an NK cell is treated with a deactivating agent for more than, less than, or exactly about 60 hours. In certain embodiments, an NK cell is treated with a deactivating agent for more than, less than, or exactly about 72 hours. In certain embodiments, an NK cell is treated with a deactivating agent for more than, less than, or exactly about 84 hours. In certain embodiments, an NK cell is treated with a deactivating agent for more than, less than, or exactly about 96 hours. In certain embodiments, an NK cell is treated with a deactivating agent for about 24 to 96 hours, about 24 to 72 hours, about 24 to 48 hours, about 36 to 84 hours, or about 48 to 72 hours.

In some embodiments, a cell (e.g., an NK cell) is treated with a deactivating agent after the cell is cultured for at least about, or about 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, or 30 days. In some embodiments, a cell (e.g., an NK cell) is treated with a deactivating agent after the cell is cultured for at least about, or about 11 days, 12 days, 13 days, 14 days, 15 days, or 16 days. In some embodiments, a cell (e.g., an NK cell) is treated with a deactivating agent after the cell is cultured for about 12 days. In some embodiments, a cell (e.g., an NK cell) is treated with a deactivating agent after the cell is cultured for about 13 days. In some embodiments, a cell (e.g., an NK cell) is treated with a deactivating agent after the cell is cultured for about 14 days. In some embodiments, a cell (e.g., an NK cell) is treated with a deactivating agent after the cell is cultured for about 15 days.

In certain embodiments, a deactivating agent is a kinase inhibitor. In some embodiments, a kinase inhibitor is a broad spectrum kinase inhibitor that may have inhibitory effects upon more than one tyrosine kinase and/or serine/threonine kinases. In certain embodiments, a deactivating agent is a mammalian (mechanistic) target of rapamycin (mTOR) inhibitor. In certain embodiments, an mTOR inhibitor is rapamycin (aka sirolimus), everolimus, and/or temsirolimus. In certain embodiments, an mTOR inhibitor is rapamycin. In certain embodiments, a deactivating agent is an FDA approved kinase inhibitor (e.g., see Robert Roskoski “Properties of FDA-approved small molecule protein kinase inhibitors: A 2021 update” Pharmalogical Research, March 2021; which is incorporated herein by reference for the purpose described herein). In certain embodiments, a deactivating agent is Dasatinib. In certain embodiments, a deactivating agent is Nilotinib.

In certain embodiments, the deactivating agent is a tyrosine kinase (TK) inhibitor. In certain embodiments, the TK inhibitor is Lorlatinib, Brigatinib, Ceritinib, Alectinib, Crizotinib, Bosutinib, Ponatinib, Nilotinib, Dasatinib, Saracatinib, Imatinib, Zanubrutinib, Acalabrutinib, Ibrutinib, Capmatinib, Pexidartinib, Dacomitinib, Osimertinib, Erlotinib, Gefitinib, Lapatinib, Afatinib, Pemigatinib, Erdafitinib, Nintedanib, Gilteritinib, Midostaurin, Tucatinib, Neratinib, Baricitinib, Ruxolitinib, Fedratinib, Tofacitinib, Ripretinib, Selumetinib, Binimetinib, Cobimetinib, Trametinib, Upadacitinib, Avapritinib, Selpercatinib, Cabozantinib, Fostamatinib, Larotrectinib, Entrectinib, Axitinib, Regorafenib, Pazopanib, Sorafenib, Lenvatinib, Vandetanib, and/or Sunitinib. In certain embodiments, the TK inhibitor is not Lorlatinib, Brigatinib, Ceritinib, Alectinib, Crizotinib, Bosutinib, Ponatinib, Nilotinib, Dasatinib, Saracatinib, Imatinib, Zanubrutinib, Acalabrutinib, Ibrutinib, Capmatinib, Pexidartinib, Dacomitinib, Osimertinib, Erlotinib, Gefitinib, Lapatinib, Afatinib, Pemigatinib, Erdafitinib, Nintedanib, Gilteritinib, Midostaurin, Tucatinib, Neratinib, Baricitinib, Ruxolitinib, Fedratinib, Tofacitinib, Ripretinib, Selumetinib, Binimetinib, Cobimetinib, Trametinib, Upadacitinib, Avapritinib, Selpercatinib, Cabozantinib, Fostamatinib, Larotrectinib, Entrectinib, Axitinib, Regorafenib, Pazopanib, Sorafenib, Lenvatinib, Vandetanib, and/or Sunitinib.

In certain embodiments, a TK inhibitor is an anaplastic lymphoma kinase (ALK) inhibitor. In certain embodiments, an ALK inhibitor is Lorlatinib, Brigatinib, Ceritinib, Alectinib, and/or Crizotinib. In certain embodiments, an ALK inhibitor is not Lorlatinib, Brigatinib, Ceritinib, Alectinib, and/or Crizotinib.

In certain embodiments, a TK inhibitor is a Break Point Cluster Tyrosine-protein kinase ABL1 fusion (BCR-Abl) inhibitor. In certain embodiments, a BCR-Abl inhibitor is Bosutinib, Ponatinib, Nilotinib, Dasatinib, Saracatinib, and/or Imatinib. In certain embodiments, the TK inhibitor is Dasatinib and/or Nilotinib. In certain embodiments the TK inhibitor is Dasatinib. In certain embodiments, the BCR-Abl inhibitor is not Bosutinib, Ponatinib, Nilotinib, Dasatinib, Saracatinib, and/or Imatinib.

In certain embodiments, a TK inhibitor is a Bruton tyrosine kinase (BTK) inhibitor. In certain embodiments, a BTK inhibitor is Zanubrutinib, Acalabrutinib, and/or Ibrutinib. In certain embodiments, a BTK inhibitor is not Zanubrutinib, Acalabrutinib, and/or Ibrutinib.

In certain embodiments, a TK inhibitor is a c-MET (a member of the MNNG HOS transforming gene family) inhibitor. In some embodiments, a c-MET inhibitor is Capmatinib. In some embodiments, a c-MET inhibitor is not Capmatinib.

In certain embodiments, a TK inhibitor is a colony stimulating factor 1 receptor (CSFR1) inhibitor. In some embodiments, a CSFR1 inhibitor is Pexidartinib. In some embodiments, a CSFR1 inhibitor is not Pexidartinib.

In certain embodiments, a TK inhibitor is an epidermal growth factor receptor (EGFR) inhibitor. In certain embodiments, an EGFR inhibitor is Dacomitinib, Osimertinib, Erlotinib, Gefitinib, Lapatinib, and/or Afatinib. In certain embodiments, an EGFR inhibitor is not Dacomitinib, Osimertinib, Erlotinib, Gefitinib, Lapatinib, and/or Afatinib.

In certain embodiments, a TK inhibitor is a fibroblast growth factor receptor (FGFR) inhibitor. In certain embodiments, a FGFR inhibitor is Pemigatinib, Erdafitinib, and/or Nintedanib. In certain embodiments, a FGFR inhibitor is not Pemigatinib, Erdafitinib, and/or Nintedanib.

In certain embodiments, a TK inhibitor is a Vascular endothelial growth factor receptor (VEGFR) inhibitor. In certain embodiments, a VEGFR inhibitor is Gilteritinib, Axitinib, Regorafenib, Pazopanib, Sorafenib, Lenvatinib, Cabozantinib, and/or Vandetanib. In certain embodiments, a VEGFR inhibitor is not Gilteritinib, Axitinib, Regorafenib, Pazopanib, Sorafenib, Lenvatinib, Cabozantinib, and/or Vandetanib.

In certain embodiments, a TK inhibitor is a fms-like tyrosine kinase 3 (FLT3) inhibitor. In certain embodiments, a FLT3 inhibitor is Midostaurin. In certain embodiments, a FLT3 inhibitor is not Midostaurin.

In certain embodiments, a TK inhibitor is a Receptor tyrosine-protein kinase erbB-2 (aka HER2) inhibitor. In some embodiments, a HER2 inhibitor is Tucatinib, Lapatinib, Afatinib, and/or Neratinib. In some embodiments, a HER2 inhibitor is not Tucatinib, Lapatinib, Afatinib, and/or Neratinib.

In certain embodiments, a TK inhibitor is a Janus Kinase 1, 2, and/or 3 (JAK) inhibitor. In some embodiments, a JAK inhibitor is Baricitinib, Ruxolitinib, Fedratinib, and/or Tofacitinib. In some embodiments, a JAK inhibitor is not Baricitinib, Ruxolitinib, Fedratinib, and/or Tofacitinib.

In certain embodiments, a TK inhibitor is a platelet-derived growth factor receptor (PDGFR) inhibitor. In certain embodiments, a PDGFR inhibitor is Ripretinib, Upadacitinib, and/or Avapritinib. In certain embodiments, a PDGFR inhibitor is not Ripretinib, Upadacitinib, and/or Avapritinib.

In certain embodiments, a TK inhibitor is a mitogen-activated protein kinase kinase 1/2 (MEK1/2) inhibitor. In some embodiments, a MEK1/2 inhibitor is Selumetinib, Binimetinib, Cobimetinib, and/or Trametinib. In some embodiments, a MEK1/2 inhibitor is not Selumetinib, Binimetinib, Cobimetinib, and/or Trametinib.

In certain embodiments, a TK inhibitor is proto-oncogene tyrosine-protein kinase receptor (RET) inhibitor. In certain embodiments, a RET inhibitor is Alectinib, Lenvatinib, Selpercatinib and/or Cabozantinib. In certain embodiments, a RET inhibitor is not Alectinib, Lenvatinib, Selpercatinib and/or Cabozantinib.

In certain embodiments, a TK inhibitor is a tyrosine-protein kinase (SYK) inhibitor. In some embodiments, a SYK inhibitor is Fostamatinib. In some embodiments, a SYK inhibitor is not Fostamatinib.

In certain embodiments, a TK inhibitor is a Trk system potassium uptake protein A, B, and/or C (TRKA/B/C) inhibitor. In some embodiments, a TRKA/B/C inhibitor is Larotrectinib, and/or Entrectinib. In some embodiments, a TRKA/B/C inhibitor is not Larotrectinib, and/or Entrectinib.

In certain embodiments, a TK inhibitor is a proto-oncogene tyrosine-protein kinase ROS (ROS1) inhibitor. In some embodiments, a ROS1 inhibitor is Crizotinib, and/or Entrectinib. In some embodiments, a ROS1 inhibitor is not Crizotinib, and/or Entrectinib.

In certain embodiments, a TK inhibitor is a mast/stem cell growth factor receptor Kit (KIT) inhibitor. In certain embodiments, a KIT inhibitor is Ripretinib, and/or Imatinib. In certain embodiments, a KIT inhibitor is not Ripretinib, and/or Imatinib.

In certain embodiments, a cell is treated with a deactivating agent at a concentration of about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nM, or any range derivable therein. In certain embodiments, a cell is treated with a deactivating agent at a concentration of about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM, or any range derivable therein. In certain embodiments, a cell is treated with a deactivating agent at a concentration of about 0.1-1, 1-5, 5-10, 10-20, 20-30, 30-40, 40-50, 50-60, 60-70, 70-80, 80-90, 90-100, 100-110, 110-120, 120-130, 130-140, 140-150, 150-160, 160-170, 170-180, 180-190, or 190-200 nM. In certain embodiments, a cell is treated with a deactivating agent at a concentration of about 0.1-1, 1-5, 5-10, 10-20, 20-30, 30-40, 40-50, 50-60, 60-70, 70-80, 80-90, 90-100, 100-110, 110-120, 120-130, 130-140, 140-150, 150-160, 160-170, 170-180, 180-190, or 190-200 μM. In certain embodiments, a cell is treated with a deactivating agent at a concentration of about 50, 100, 150, 200, 250, 300, 350, 400, 450, 500, 550, 600, 650, 700, 750, 800, 850, 900, 950, 1,000, 1,050, 1,100, 1,150, 1,200, 1,250, 1,300, 1,350, 1,400, 1,450, 1,500, 1,550, 1,600, 1,650, 1,700, 1,750, 1,800, 1,850, 1,900, 1,950, 2,000, 2,500, 3,000, 3,500, 4,000, 4,500, 5,000, 5,500, 6,000, 6,500, 7,000, 7,500, 8,000, 8,500, 9,000, 9,500, or 10,000 nM, or any range derivable therein. In certain embodiments, a cell is treated with a deactivating agent at a concentration of about 50, 100, 150, 200, 250, 300, 350, 400, 450, 500, 550, 600, 650, 700, 750, 800, 850, 900, 950, 1,000, 1,050, 1,100, 1,150, 1,200, 1,250, 1,300, 1,350, 1,400, 1,450, 1,500, 1,550, 1,600, 1,650, 1,700, 1,750, 1,800, 1,850, 1,900, 1,950, 2,000, 2,500, 3,000, 3,500, 4,000, 4,500, 5,000, 5,500, 6,000, 6,500, 7,000, 7,500, 8,000, 8,500, 9,000, 9,500, or 10,000 μM, or any range derivable therein.

In certain embodiments, cells are deactivated by treating the cells with a deactivating agent. In some embodiments, cells are deactivated by treating the cells with a deactivating agent prior to storage, for example but not limited to, cryopreservation, and under conditions suitable for the cells to become deactivated. In some embodiments, a deactivating agent is at a concentration of at least or about 2000, 1000, 500, 200, 100, 50, 40, 30, 25, 24, 23, 22, 21, 20, 19, 18, 17, 16, 15, 14, 13, 12, 11, 10, 9, 8, 7, 6, 5, 4, 3, 2, or 1 mM, or any range derivable therein. In some embodiments, a deactivating agent is at a concentration of at least about, or about 11 mM. In some embodiments, a deactivating agent is at a concentration of at least about, or about 10 mM. In some embodiments, a deactivating agent is at a concentration of at least about, or about 9 mM. In some embodiments, a deactivating agent is in a suitable aqueous or non-aqueous solvent. In some embodiments, a deactivating agent is in a suitable non-aqueous solvent. In some embodiments, a deactivating agent is in DMSO. In some embodiments, a deactivating agent and/or any solvent are stored under cold conditions.

In certain embodiments, cells are treated by a deactivating agent based on the total volume of cell culture. In some embodiments, for every 1 mL of cell culture volume, at least or about 0.001, 0.01, 0.05, 0.1, 0.15, 0.2, or 0.3 μL of a deactivating agent is added to the cell culture. In some embodiments, for every 1 mL of cell culture volume, at least about, or about 0.05 μL of a deactivating agent at a concentration of at least about, or about 10 mM in DMSO is added to the cell culture. In some embodiments, for every 1 mL of cell culture volume, at least about, or about 0.1 μL of a deactivating agent at a concentration of at least about, or about 10 mM in DMSO is added to the cell culture. In some embodiments, for every 1 mL of cell culture volume, at least about, or about 0.15 μL of a deactivating agent at a concentration of at least about, or about 10 mM in DMSO is added to the cell culture. In some embodiments, cells are treated by a deactivating agent at a final concentration of at least about, or about 0.05 μM, 0.1 μM, 0.5 μM, 1 μM, 5 μM, 10 μM, 50 μM, or 100 μM, or any range derivable therein. In some embodiments, cells are treated by a deactivating agent at a final concentration of at least about, or about 0.5 μM. In some embodiments, cells are treated by a deactivating agent at a final concentration of at least about, or about 1 μM. In some embodiments, cells are treated by a deactivating agent at a final concentration of at least about, or about 5 μM.

In certain embodiments, treatment of a cell (e.g., an NK cell) with a deactivating agent results in an increased expression of one or more of C-kit, CCR-5, CD62L and/or CXCR4, and/or decreased expression of one or more of NKG2D, DNAM, OX-40, TRAIL, HLA-DR, CD2, CD25, ICOS, and/or CD95 relative to an activated NK cell.

### E. Harvesting Cells for Cryopreservation

In some embodiments, following deactivation, cells are harvested before being placed in cryopreservation media for storage (e.g., cryopreservation).

In certain embodiments, wash media may be used to wash the cells following deactivation. In some embodiments, the wash media comprises HSA PlasmaLyte-A buffer at a concentration of about 0.1%, 0.2%, 0.3%, 0.4%, 0.5%, 0.6%, 0.7%, 0.8%, 0.9%, or 1.0% HSA, or any range derivable therein. HSA PlasmaLyte-A buffer may be prepared by adding a desired amount of HSA at a concentration of about 25% into 1 L of PlasmaLyte-A. For example, in some embodiments, 0.5% HSA PlasmaLyte-A buffer may be prepared by adding 20 mL of 25% HSA to 1 L of Plasma-Lyte A.

In certain embodiments, cells may be washed by wash media using centrifugation under mild and non-damaging conditions, such as a low centrifugation speed, with no or low brake, and/or for a short centrifugation time. In some embodiments, as would be understood by the skilled person, different combinations of centrifugation parameters may be employed to achieve cell washing while reducing potential damages to the cells. In some embodiments, centrifugation and handling of cells may be performed under sterile conditions using sterile containers and/or equipment.

In certain embodiments, cells and culture media may be used for one or more quality control tests. The one or more quality control tests may include the following aspects, for examples, cell count, cell viability, microorganisms (e.g., mycoplasma measured by PCR), viruses (e.g., adventitious virus), cytokines (e.g., IL-15 and IL-15 by Elisa), immunophenotyping, endotoxins, vector copy number, replication-competent retrovirus (RCR, e.g., measured by QPCR), and/or residual bead.

In certain embodiments, cells may be washed before being placed in cryopreservation media. In some embodiments, an exemplary method of washing cells comprises: harvesting cells from one or more cell culture flasks and pooling into one or more appropriately labelled sterile centrifuge tubes; gently re-suspending the pooled cell suspension; washing cells, comprising centrifuging the cells using mild and/or non-damaging centrifuge settings, aseptically aspirating supernatant and discarding, gently re-suspending cell pellet using a suitable buffer (e.g., 0.5% HSA PlasmaLyte buffer); collecting quality control samples; and calculating total viable cells per mL from quality control count and the total viable cells recovery post harvesting.

In certain embodiments, cells may be placed in cryopreservation media with or without washing. In some embodiments, cell pellets may be harvested by centrifuging using mild and/or non-damaging centrifuge settings, and the cell pellets may be gently dispersed in freeze media and transferred to a refrigerator (e.g., at about 4° C.) for a period of time.

### F. Cryopreservation Media

In some embodiments, following deactivation and/or harvesting, cells are placed in a cryopreservation media for storage (e.g., cryopreservation).

In certain embodiments, at least one cytokine and/or at least one growth factor may be added to a cryopreservation media. In some embodiments, a cryoprotectant may be dimethyl sulfoxide (DMSO), glycerin, glycerol, hydroxyethyl starch, dextran trehalose, or a combination thereof. In some embodiments, a non-serum alternative may comprise platelet lysate and/or a blood product lysate or human or animal serum albumin. In some cases, the at least one cytokine (which may be interleukin (IL)-1, IL-2, IL-3, IL-4, IL-6, IL-7, IL-9, IL-10, IL-12, IL-13, IL-15, IL-17, IL-18, IL-21, IL-22, IL-23, interferon, tumor necrosis factor, stem cell factor, FLT3-ligand, APRIL, thrombopoietin, erythropoietin, or a combination thereof) is a natural protein, a recombinant protein, a synthetic protein, or a mixture thereof.

In particular embodiments, a cryopreservation media suitable for cryopreserving deactivated cells of the present disclosure may comprise dimethyl sulfoxide (DMSO); serum (including human serum); and one or more cytokines of any kind. In specific embodiments, any one or more components of the cryopreservation media are natural proteins, which may also be referred to as endogenous or recombinant proteins. In specific cases the endogenous proteins are the one or more cytokines. The cryopreservation media may also comprise one or more FDA-approved agents, and the one or more FDA-approved agents may be the one or more cytokines, in certain cases.

In particular embodiments, cryopreservation media compositions comprise, consist of, or consist essentially of at least one cryoprotectant, at least one serum (or non-serum alternative to serum), and at least one cytokine and/or at least one growth factor. Examples of cryoprotectants include dimethyl sulfoxide (DMSO), glycerin, glycerol, hydroxyethyl starch, or a combination thereof. For the composition, the non-serum alternative may comprise platelet lysate and/or a blood product lysate and/or human serum albumin and/or animal serum albumin. The human serum may be human AB serum. Any cytokine may be a natural protein, a recombinant protein, a synthetic protein, or a mixture thereof, including at least one cytokine being a Food and Drug Administration (FDA)-approved cytokine. In specific cases, the composition comprises two or more cytokines. Merely as examples, at least one cytokine is IL-1, IL-2, IL-3, IL-4, IL-6, IL-7, IL-9, IL-10, IL-12, IL-13, IL-15, IL-17, IL-18, IL-21, IL-22, interferon, tumor necrosis factor, stem cell factor, FLT3-ligand, APRIL, or a combination thereof.

In particular cases, the one or more cytokines include IL-2, IL-15, IL-12, IL-18, and/or IL-21. The cells may be suspended in GMP cryopreservation medium comprising DMSO (e.g., 1-10%, such as 1, 2, 3, 4, 5, 6, 7, 8, 9 or 10%, particularly 5%), 95% Human AB Serum (e.g., 90-99%, such as 91, 92, 93, 94, 95, 96, 97, 98, or 99%, particularly 95%), Platelet lysate (e.g., 90-99%, such as 91, 92, 93, 94, 95, 96, 97, 98, or 99%, particularly 95%), IL-2 (e.g., 50-500 U/mL, such as 100, 200, 300, 400, 500, 1000, or 5000 U/mL, particularly 400 U/mL), IL-15 (5-500 ng/ml) and/or IL-21 (e.g., 1-500 ng/mL, such as 10, 20, 30, 40, 50, 100, or 500 ng/mL, particularly 20 ng/mL). In particular cases, the cells are frozen in liquid nitrogen using a rate controlled method.

In particular embodiments, the cryoprotectant comprises a particular amount of the composition; in specific aspects, the cryoprotectant comprises 4-6% of the composition or 5-10% of the composition; in specific cases, the cryoprotectant comprises 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10% of the composition. The serum may comprise a particular amount of the composition, such as comprising 5-99, 5-90, 5-85, 5-80, 5-75, 5-70, 5-65, 5-60, 5-55, 5-50, 5-45, 5-40, 5-35, 5-30, 5-25, 5-20, 5-15, 5-10, 10-99, 10-90, 10-85, 10-80, 10-75, 10-70, 10-65, 10-60, 10-55, 10-50, 10-45, 10-40, 10-35, 10-30, 10-25, 10-20, 10-15, 25-99, 25-90, 25-85, 25-08, 25-75, 25-70, 25-65, 25-60, 25-55, 25-50, 25-45, 25-40, 25-35, 25-30, 50-99, 50-90, 50-85, 50-80, 50-75, 50-70, 40-65, 50-60, or 50-55% of the composition. The platelet lysate may comprise a certain amount of the composition, such as 5-99, 5-90, 5-85, 5-80, 5-75, 5-70, 5-65, 5-60, 5-55, 5-50, 5-45, 5-40, 5-35, 5-30, 5-25, 5-20, 5-15, 5-10, 10-99, 10-90, 10-85, 10-80, 10-75, 10-70, 10-65, 10-60, 10-55, 10-50, 10-45, 10-40, 10-35, 10-30, 10-25, 10-20, 10-15, 25-99, 25-90, 25-85, 25-08, 25-75, 25-70, 25-65, 25-60, 25-55, 25-50, 25-45, 25-40, 25-35, 25-30, 50-99, 50-90, 50-85, 50-80, 50-75, 50-70, 40-65, 50-60, or 50-55% of the composition. In specific aspects, the platelet lysate comprises 95% of the composition.

In embodiments wherein the composition comprises IL-2, it may be present at a concentration of 1-5000, 1-4000, 1-3000, 1-2000, 10-1000, 100-5000, 100-4000, 100-3000, 100-1000, 100-1000, 100-500, 500-5000, 500-4000, 500-3000, 500-2000, 500-1000, 1000-5000, 1000-4000, 1000-3000, 1000-2000, or 2000-5000 U/mL, including specifically at 100, 200, 300, 400, or 500 U/mL. In embodiments wherein the composition comprises IL-21, it may be present at a concentration of 10-3000, 10-2500, 10-2000, 10-1000, 10-500, 100-3000, 100-2000, 100-1000, 500-3000, 500-2000, 500-1000, or 1000-3000 ng/mL, including specifically being present at a concentration of 10, 15, 20, or 25 ng/mL. In specific cases, the IL-15 is present in the composition at a concentration of 10-2000, 10-1000, 10-500, 100-2000, 100-1000, 100-500, 500-2000, 500-1000, or 1000-2000 ng/mL.

For cryopreservation, as one example, the deactivated cells (such as NK cells for adoptive therapy, including cord blood NK cells) may be suspended in a GMP cryopreservation medium comprising, for example, 5% DMSO, 95% Human AB Serum, 400 units IL-2/ml, and 20ng IL-21/ml. They may be frozen using dry ice, liquid nitrogen, a non-liquid nitrogen freezer, via dump freezing, a rate-controlled freezing method (e.g., using a control rate freezer), and/or a non-rate controlled freezing method, for example. In some embodiments, after freezing, the deactivated cells may be stored in vapor phase in liquid nitrogen storage unit.

In some embodiments, a cryopreservation medium comprises glucose, a pH indicator, one or more salts, one or more amino acids, and one or more vitamins. Examples of pH indicators include at least phenol red, bromophenol blue, methyl orange, bromocresol purple, Congo red, and so forth. Examples of salts include at least sodium chloride, sodium bicarbonate, disodium phosphate, potassium chloride, magnesium sulfate, calcium nitrate, or a combination thereof. Examples of amino acids include glutamine, arginine, asparagine, cysteine, leucine, isoleucine, lysine, serine, aspartic acid, glutamic acid, hydroxyproline, proline, threonine, tyrosine, valine, histidine, methionine, phenylalanine, glycine, tryptophan, reduced glutathione, or a combination thereof. In some embodiments, one or more amino acids are greater in amount in the media than one or more other amino acids, whereas one or more other amino acids may be in the same amount in the media. For example, glutamine may or may not be greatest in amount in the media, followed by arginine. Asparagine, cysteine, leucine, isoleucine, or a combination thereof may or may not be substantially the same amount in the media. Aspartic acid, glutamic acid, hydroxyproline, proline, threonine, tyrosine, valine, or a combination thereof may or may not be substantially the same amount in the media. Histidine, methionine, phenylalanine, or a combination thereof may or may not be substantially the same amount in the media. One or more specific vitamins may be present in the media, including i-inositol; choline chloride; para-aminobenzoic acid, folic acid, nicotinamide, pyridoxine hydrochloride, thiamine hydrochloride; calcium pantothenate; biotin; riboflavin; cyanocobalamin; or a combination thereof may be present in the media. The vitamins may or may not be present in the media at specific amounts. For example, i-inositol may be present in the greatest amount, followed by choline chloride. Certain vitamins may be substantially equal in the media, including para-aminobenzoic acid, folic acid, nicotinamide, pyridoxine hydrochloride, thiamine hydrochloride, or a combination thereof, in some cases. Biotin and riboflavin may or may not be essentially equal in amount in the media. Cyanocobalamin may or may not be present as the least amount of any vitamin in the media.

In some embodiments, the cells may be cultured in a media that is substantially similar or identical to RPMI 1640 medium, also known as RPMI medium, that is a growth medium developed by Moore et al. (Moore G E, Gerner R E, Franklin H A (1967). “Culture of normal human leukocytes”. JAMA. 199 (8): 519-524) at Roswell Park Memorial Institute.

In a specific example, one liter of RPMI 1640 contains or comprises the following: Glucose (2 g); pH indicator (phenol red, 5 mg); Salts (6 g sodium chloride, 2 g sodium bicarbonate, 1.512 g disodium phosphate, 400 mg potassium chloride, 100 mg magnesium sulfate, and 100 mg calcium nitrate); Amino acids (300 mg glutamine; 200 mg arginine; 50 mg each asparagine, cystine, leucine, and isoleucine; 40 mg lysine hydrochloride; 30 mg serine; 20 mg each aspartic acid, glutamic acid, hydroxyproline, proline, threonine, tyrosine, and valine; 15 mg each histidine, methionine, and phenylalanine; 10 mg glycine; 5 mg tryptophan; and 1 mg reduced glutathione); and Vitamins (35 mg i-inositol; 3 mg choline chloride; 1 mg each para-aminobenzoic acid, folic acid, nicotinamide, pyridoxine hydrochloride, and thiamine hydrochloride; 0.25 mg calcium pantothenate; 0.2 mg each biotin and riboflavin; and 0.005 mg cyanocobalamin).

In specific embodiments, the composition comprises: a) one or more of platelet lysate, PlasmaLyte, and Roswell Park Memorial Institute (RPMI) media; (b) one or more of dextran that can be formulated in dextrose or in saline (for example), albumin, and DMSO; and (c) one or more of IL-2, IL-15, and IL-21. In specific embodiments, any composition comprises platelet lysate between 50% and 90% of the composition, including about 50% of the composition or about 90% of the composition. In cases wherein PlasmaLyte is utilized, it may be between about 32.5% and 70% of the composition, including at about 32.5%, 35%, 50%, or 70% of the composition. The RPMI may be between 32.5% and 50% of the composition, including at about 32.5%, 35%, or 50% of the composition. In cases wherein dextran is utilized, the dextran may be about 25-40% of the composition, including at about 25% or about 40% of the composition. In cases wherein albumin is utilized, it may be about 1-99% of the composition, including at about 20% of the composition. In cases wherein DMSO is utilized, it may be about 5-7.5% of the composition, including specifically at about 5% or 7.5% of the composition.

In some embodiments, the cells of the disclosure may be preserved in the following particular formulations for cryopreservation at any time. In some embodiments, the cells of the disclosure may be preserved in the following particular formulations for cryopreservation following treatment with a deactivating agent. Examples of particular formulations with certain concentrations as described below may be utilized.

TABLE 1
Exemplary cryopreservation media

50% RPMI; 25% dextran; 20% human albumin, 5% DMSO
50% RPMI; 25% dextran; 20% human albumin, 5% DMSO + IL-2/IL-15
35% RPMI; 40% dextran; 20% human albumin, 5% DMSO
35% RPMI; 40% dextran; 20% human albumin, 5% DMSO + IL-2/IL-15
32.5% RPMI; 40% dextran; 20% human albumin, 7.5% DMSO
32.5% RPMI; 40% dextran; 20% human albumin, 7.5% DMSO + IL-2/IL-15
50% PlasmaLyte; 25% dextran; 20% human albumin, 5% DMSO
50% PlasmaLyte; 25% dextran; 20% human albumin, 5% DMSO + IL-2/IL-15
35% PlasmaLyte; 40% dextran; 20% human albumin, 5% DMSO
35% PlasmaLyte; 40% dextran; 20% human albumin, 5% DMSO + IL-2/IL-15
32.5% PlasmaLyte; 40% dextran; 20% human albumin, 7.5% DMSO
32.5% PlasmaLyte; 40% dextran; 20% human albumin, 7.5% DMSO + IL-2/IL-15
70% PlasmaLyte; 25% dextran; 5% DMSO + IL-2/IL-15
90% PLT Lys + 10% DMSO + IL-2/IL-15
50% PLT lys + 25% dextran + 20% human albumin + 5% DMSO + IL-2/IL-15
50% AB serum + 25% dextran + 20% human albumin + 5% DMSO + IL-2/IL-15
50% PLT lys + 25% dextran + 20% human albumin + 5% DMSO + IL-2/IL-21
50% RPMI + 25% dextran + 20% human albumin + 5% DMSO + IL-2/IL-21
50% PlasmaLyte + 25% dextran + 20% human albumin + 5% DMSO + IL-2/IL-21
50% AB serum + 25% dextran + 20% human albumin + 5% DMSO + IL-2/IL-21
50% PLT Lys; 25% Dextran in NACL; 20% human albumin; 5% DMSO + IL-2/IL-15
50% PLT Lys; 25% Dextran in Dextrose; 20% human albumin; 5% DMSO + IL-2/IL-15
25% PLT Lys; 50% Dextran in NACL; 20% human albumin; 5% DMSO + IL-2/IL-15
25% PLT Lys; 50% Dextran in Dextrose; 20% human albumin; 5% DMSO + IL-2/IL-15
25% Dextran in NACL; 70% human albumin; 5% DMSO + IL-2/IL-15
25% Dextran in Dextrose; 70% human albumin; 5% DMSO + IL-2/IL-15
50% Dextran in NACL; 45% human albumin; 5% DMSO + IL-2/IL-15
50% Dextran in Dextrose; 45% human albumin; 5% DMSO + IL-2/IL-15
50% PlasmaLyte; 45% human albumin; 5% DMSO + IL-2/IL-15
25% PlasmaLyte; 70% human albumin; 5% DMSO + IL-2/IL-15
Crystor Cell preservation Media CS10 (1% to 100%)
Crystor Cell preservation Media CS10 (1% to 100%) + IL-2/15

TABLE 2
Exemplary cryopreservation media with particular concentrations

50% Platelet lysate; 25% Dextran in NaCL; 20% human albumin; 5% DMSO; plus 10-200iu
of interleukin 2 and/or 1-10 ng/ml of interleukin 15
50% Platelet lysate; 25% Dextran in Dextrose; 20% human albumin; 5% DMSO; plus 10-
200iu of interleukin 2 and/or 1-10 ng/ml of interleukin 15
25% Platelet lysate; 50% Dextran in NaCL; 20% human albumin; 5% DMSO; plus 10-200iu
of interleukin 2 and/or 1-10 ng/ml of interleukin 15
25% Platelet lysate; 50% Dextran in Dextrose; 20% human albumin; 5% DMSO; plus 10-
200iu of interleukin 2 and/or 1-10 ng/ml of interleukin 15
25% Dextran in NaCL; 70% human albumin; 5% DMSO; plus 10-200iu of interleukin 2
and/or 1-10 ng/ml of interleukin 15
25% Dextran in Dextrose; 70% human albumin; 5% DMSO; plus 10-200iu of interleukin 2
and/or 1-10 ng/ml of interleukin 15
50% Dextran in NaCL; 45% human albumin; 5% DMSO; plus 10-200iu of interleukin 2
and/or 1-10 ng/ml of interleukin 15
50% Dextran in Dextrose; 45% human albumin; 5% DMSO; plus 10-200iu of interleukin 2
and/or 1-10 ng/ml of interleukin 15
50% PlasmaLyte; 45% human albumin; 5% DMSO; plus 10-200iu of interleukin 2 and/or 1-
10 ng/ml of interleukin 15
25% PlasmaLyte; 70% human albumin; 5% DMSO; plus 10-200iu of interleukin 2 and/or 1-
10 ng/ml of interleukin 15
90% Platelet lysate, 10% DMSO
90% Platelet lysate, 10% DMSO plus 10-200iu of interleukin 2 and/or 1-10 ng/ml of
interleukin 15
Crystor Cell preservation Media CS10 (1% to 100%)
Crystor Cell preservation Media CS10 (1% to 100%) + IL-2

### III. Cells for Storage

Cells to be stored (e.g., cryopreserved) may be of any kind including prokaryotic or eukaryotic, but in specific embodiments the cells are mammalian cells. In particular embodiments, cells are deactivated prior to storage (e.g., treated with one or more deactivating agents pre-cryopreservation). In more specific embodiments, the mammalian cells are immune cells. In even more specific embodiments, the immune cells are NK cells. In some embodiments, the NK cells may be derived from any source as described herein. In particular embodiments, the NK cells are derived from human CB.

In some embodiments, the mammalian cells may be utilized for research or therapeutic purposes of any kind. In specific embodiments, the cells are immune cells of any kind, including NK cells, T cells, NK T cells, PBMCs, antigen presenting cells (APCs), B cells, mononuclear cells, dendritic cells, macrophages, monocytes, neutrophils, induced pluripotent stem cells (iPSCs), hematopoietic stem cells, or any cell derived from hematopoietic stem cells, iPSCs, and/or MSCs, differentiated or committed cells from any organ, any fibroblasts, and so forth. The cells may or may not be stem cells, in some examples.

As described herein, cells are deactivated prior to storage (e.g., cryopreservation). Additionally, in some embodiments, cells are modified prior to and/or after deactivation and/or storage. For example, the cells may be transfected or transduced with a vector or electroporated with a plasmid that encodes a particular gene product, such as a gene product that imparts a therapeutic activity to the cells. In specific embodiments, the cells are transfected or transduced or electroporated with one or more antigen receptors, including T cell receptors (TCRs) or chimeric antigen receptors (CARs), cytokines, homing receptors or any other genes. In specific cases, the cells are CAR-expressing immune cells, such as CAR-expressing NK cells. In specific cases, the cells are gene edited.

In certain embodiments, cells may be stored at various concentrations and/or in various volumes in suitable containers. In some embodiments, cell may be stored at a concentration of at least about, or about 3×10⁴, 3×10⁵, 3×10⁶, 5×10⁶, 1×10⁷, 2.5×10⁷, 5×10⁷, or 1×10⁸ cells per mL, or any range derivable therein. In some embodiments, cell may be stored in one or more vials with the size of at least about, or about 0.5 mL, 1 mL, 2 mL, 6 mL, 20 mL, 50 mL, 100 mL, 150 mL, or 200 mL, or any range derivable therein.

In certain embodiments, NK cells are derived from human peripheral blood mononuclear cells (PBMC), unstimulated leukapheresis products (PBSC), human embryonic stem cells (hESCs), induced pluripotent stem cells (iPSCs), bone marrow, or umbilical cord blood by methods well known in the art. Specifically, the NK cells may be isolated from cord blood (CB), peripheral blood (PB), bone marrow, or stem cells. In particular embodiments, the immune cells are isolated from pooled CB. The CB may be pooled from 2, 3, 4, 5, 6, 7, 8, 10, or more units. The immune cells may be autologous or allogeneic. The isolated NK cells may be completely matched, completely mismatched, haplotype matched (half matched) or more than haplotype but less than completely matched with the subject to be administered the cell therapy. NK cells can be detected by specific surface markers, such as CD16 and CD56 in humans.

In certain aspects, the starting population of NK cells is obtained by isolating mononuclear cells using ficoll density gradient centrifugation. The cell culture may be depleted of any cells expressing CD3, CD14, and/or CD19 cells and may be characterized to determine the percentage of CD56⁺/CD3⁻ cells or NK cells. They may also be subjected to positive selection with CD56 or other specific NK cell antibodies, in certain procedures.

The cells may be expanded in the presence of APCs, such as universal APCs and/or artificial APCs. The expansion may be for about 2-30 days, or longer, such as 3-20 days, particularly 12-16 days, such as 12, 13, 14, 15, 16, 17, 18, or 19 days, specifically about 14 days. The NK cells and APCS may be present at a ratio of about 3:1-1:3, such as 2:1, 1:1, 1:2, specifically about 1:2. The expansion culture may further comprise cytokines to promote expansion, such as IL-2, IL-2, IL-15, IL-21, and/or IL-18. The cytokines may be present at a concentration of about 10-500 U/mL, such as 100-300 U/mL, particularly about 200 U/mL. The cytokines may be replenished in the expansion culture, such as every 2-3 days. The APCs may be added to the culture at least a second time, such as after transgene (e.g., CAR, TCR, cytokine, etc.) transduction. In particular embodiments, the cytokines are present in the cryopreservation medium at a level that avoids providing a therapeutic effect to the individual upon receipt of the cells, for example if and when the medium is included with the cells upon administering them to a subject. The cells may be comprised in at least some of the cryopreservation medium either because of residual medium upon preparation of the cells for administering, or the cells may be comprised in at least some of the cryopreservation medium by intended design. Following thawing of the cells, the cells may or may not be washed prior to administering to a subject.

In some specific embodiments, the starting population of cells are MNCs isolated from a single CB unit by ficoll density gradient. The cells can then be washed and depleted of the CD3, CD14 and CD19 positive cells, such as by using the CliniMACS immunomagnetic beads (Miltenyi Biotec). The unlabeled, enriched CB-NK cells can be collected, washed with CliniMACS buffer, counted, and combined with irradiated (e.g., 100 Gy) APCs, such as in a 1:2 ratio. The cell mixture (e.g., 1×10⁶ cells/mL) may be transferred to cell culture flasks containing NK Complete Medium (e.g., 90% Stem Cell Growth Medium, 10% FBS, 2 mM L-glutamine) and IL-2, such as 50-500, such as 100-300, such as 200 U/mL. The cells can be incubated at 37° C. in 5% CO₂. On Day 3, a media change may be performed by collecting the cells by centrifugation and resuspending them in NK Complete Medium (e.g., 1×10⁶ cells/mL) containing IL-2, such as 50-500, such as 100-300, such as 200 U/mL. The cells may be incubated at 37° C. in 5% CO₂. On Day 5, the number of wells needed for RetroNectin transduction can be determined by the number of CB-NK cells in culture. The RetroNectin solution may be plated to wells of 24-well culture plates. The plates can be sealed and stored in a 4° C. refrigerator.

In some specific embodiments, on Day 6, a 2^{nd} NK selection as described on Day 0 can be performed prior to transduction of the CB-NK cells. The cells can be washed with CliniMACS buffer, centrifuged, and resuspended in NK Complete Medium at 0.5×10⁶/mL with IL-2, such as 100-1000, particularly 600 U/mL. The RetroNectin plates can then be washed with NK complete medium and incubated at 37° C. until use. The NK complete medium in each well can be replaced with retroviral supernatant, followed by centrifugation of plates at 32° C. The retroviral supernatant may then be aspirated and replaced with fresh retroviral supernatant. The CB-NK cell suspension containing 0.5×10⁶ cells and IL-2, 600 U/mL, may be added to each well, and the plates may be centrifuged. The plates can then be incubated at 37° C. with 5% CO₂. On Day 9, the CAR transduced CB-NK cells can be removed from the transduction plates, collected by centrifugation and stimulated with irradiated (e.g., 100 Gy) aAPCs, such as in a ratio of 1:2, in NK Complete Medium with IL-2, 200 U/mL. The cell culture flasks were incubated at 37° C. with 5% CO₂. On Day 12, media change may be performed. On Day 14, the cells can be collected by centrifugation, the supernatant may be aspirated and the cells can be resuspended in fresh NK Complete Medium containing IL-2, 200 U/mL. The cell culture flasks are incubated at 37° C. with 5% CO₂. If more than 1×10⁵ CD3+ cells/kg are present, a magnetic immunodepletion of CD3+ cells may be performed using CliniMACS CD3 Reagent. On Day 14 or 15, the cells are harvested and the cells are then deactivated by treatment with a deactivation agent as described herein. Following a suitable period of deactivation, the cells can be prepared for storage (e.g., cryopreservation). After any suitable period of storage, the cells can be thawed and utilized for any suitable purpose, such as but not limited to immunotherapy and/or additional modifications.

Expanded NK cells can secrete type I cytokines, such as interferon-γ, tumor necrosis factor-α and granulocyte-macrophage colony-stimulating factor (GM-CSF), which activate both innate and adaptive immune cells as well as other cytokines and chemokines. The measurement of these cytokines can be used to determine the activation status of NK cells. In addition, other methods known in the art for determination of NK cell activation may be used for characterization of the NK cells of the present disclosure.

In specific embodiments, the cells are manipulated to express one or more engineered antigen receptors (including one or more chimeric antigen receptors and/or one or more engineered TCRs); one or more cytokines; one or more suicide genes; CD47; HLA-G; HLA-E; or a combination thereof.

### A. Engineered Antigen Receptors

In some embodiments, the cells to be deactivated and cryopreserved are manipulated to express one or more engineered antigen receptors, either before deactivation and cryopreservation and/or after deactivation, cryopreservation, and subsequent thawing.

In some embodiments, the cells (e.g., NK cells) may be genetically modified to express one or more engineered antigen receptors, including at least one or more chimeric antigen receptors (CARs) and/or one or more TCRs. In specific embodiments, the engineered antigen receptors are directed to target one or more cancer antigens.

In some embodiments, the engineered antigen receptors include CARs, including activating or stimulatory CARs, costimulatory CARs (see WO 2014/055668), and/or inhibitory CARs (iCARs, see Fedorov et al., 2013). The CARs generally include an extracellular antigen (or ligand) binding domain linked to one or more intracellular signaling components, in some aspects via linkers and/or transmembrane domain(s). Such molecules typically mimic or approximate a signal through a natural antigen receptor, a signal through such a receptor in combination with a costimulatory receptor, and/or a signal through a costimulatory receptor alone.

In some embodiments, the cells (e.g., NK cells) may be modified to encode at least one CAR, and the CAR may be first generation, second generation, or third or a subsequent generation, for example. The CAR may or may not be bispecific for two or more different antigens. The CAR may comprise one or more costimulatory domains. NK cells may also be modified to express a receptor to enhance their binding to an antibody, such as CD16, CD32 and/or CD64 receptor. Each costimulatory domain may comprise the costimulatory domain of any one or more of, for example, members of the TNFR superfamily, CD28, CD137 (4-1BB), CD134 (OX40), DAP10, DAP12, CD27, CD2, CD5, ICAM-1, LFA-1 (CD11a/CD18), Lck, TNFR-I, TNFR-II, Fas, CD30, CD27, NKG2D, 2B4M, CD40 or combinations thereof, for example. In specific embodiments, the CAR comprises CD3zeta. In certain embodiments, the CAR lacks one or more specific costimulatory domains; for example, the CAR may lack 4-1BB and/or lack CD28.

In particular embodiments, the CAR polypeptide in the cells comprises an extracellular spacer domain that links the antigen binding domain and the transmembrane domain, and this may be referred to as a hinge. Extracellular spacer domains may include, but are not limited to, Fc fragments of antibodies or fragments or derivatives thereof, hinge regions of antibodies or fragments or derivatives thereof, CH2 regions of antibodies, CH3 regions antibodies, artificial spacer sequences or combinations thereof. Examples of extracellular spacer domains include but are not limited to CD8-alpha hinge, CD28, artificial spacers made of polypeptides such as Gly3, or CH1, CH3 domains of IgGs (such as human IgG1 or IgG4). In specific cases, the extracellular spacer domain may comprise (i) a hinge, CH2 and CH3 regions of IgG4, (ii) a hinge region of IgG4, (iii) a hinge and CH2 of IgG4, (iv) a hinge region of CD8-alpha or CD4, (v) a hinge, CH2 and CH3 regions of IgG1, (vi) a hinge region of IgG1 or (vii) a hinge and CH2 of IgG1, (viii) a hinge region of CD28, or a combination thereof. In specific embodiments, the hinge is from IgG1 and in certain aspects the CAR polypeptide comprises a particular IgG1 hinge amino acid sequence or is encoded by a particular IgG1 hinge nucleic acid sequence.

In some embodiments, the transmembrane domain in the CAR may be derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 zeta, CD3 epsilon, CD3 gamma, CD3 delta, CD45, CD4, CD5, CD8, CD9, CD 16, CD22, CD30, CD33, CD37, CD64, CD80, CD86, CD 134, CD137, CD154, ICOS/CD278, GITR/CD357, NKG2D, and DAP molecules, such as DAP10 or DAP12. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine may be found at each end of a synthetic transmembrane domain.

Certain embodiments of the present disclosure concern the use of nucleic acids, including nucleic acids encoding an antigen-specific CAR polypeptide, including a CAR that has been humanized to reduce immunogenicity (hCAR), comprising an intracellular signaling domain, a transmembrane domain, and an extracellular domain comprising one or more signaling motifs. In certain embodiments, the CAR may recognize an epitope comprising the shared space between one or more antigens. In certain embodiments, the binding region can comprise complementary determining regions of a monoclonal antibody, variable regions of a monoclonal antibody, and/or antigen binding fragments thereof. In another embodiment, that specificity is derived from a peptide (e.g., cytokine) that binds to a receptor.

It is contemplated that the human CAR nucleic acids may be human genes used to enhance cellular immunotherapy for human patients. In a specific embodiment, the invention includes a full-length CAR cDNA or coding region. The antigen binding regions or domain can comprise a fragment of the V_{H} and V_{L} chains of a single-chain variable fragment (scFv) derived from a particular human monoclonal antibody, such as those described in U.S. Pat. No. 7,109,304, incorporated herein by reference. The fragment can also be any number of different antigen binding domains of a human antigen-specific antibody. In a more specific embodiment, the fragment is an antigen-specific scFv encoded by a sequence that is optimized for human codon usage for expression in human cells. The CAR may be bi-specific for two non-identical antigenic targets or tri-specific for three non-identical antigenic targets, and so forth.

The arrangement could be multimeric, such as a diabody or multimers. The multimers are most likely formed by cross pairing of the variable portion of the light and heavy chains into a diabody. The hinge portion of the construct can have multiple alternatives from being totally deleted, to having the first cysteine maintained, to a proline rather than a serine substitution, to being truncated up to the first cysteine. The Fc portion can be deleted. Any protein that is stable and/or dimerizes can serve this purpose. One could use just one of the Fc domains, e.g., either the CH2 or CH3 domain from human immunoglobulin. One could also use the hinge, CH2 and CH3 region of a human immunoglobulin that has been modified to improve dimerization. One could also use just the hinge portion of an immunoglobulin. One could also use portions of CD8alpha.

In certain embodiments, the CAR may be co-expressed with one or more cytokines to improve persistence when there is a low amount of tumor-associated antigen. For example, the CAR may be co-expressed with one or more cytokines, such as IL-7, IL-2, IL-15, IL-12, IL-23, IL-18, IL-21, IL-7, GMCSF, or a combination thereof. In some embodiments, the cells (e.g., NK cells) expressing the CAR are engineered to express one or more heterologous cytokines and/or are engineered to upregulate normal expression of one or more heterologous cytokines. The cells may or may not be transduced or transfected for one or more cytokines on the same vector as other genes. In certain embodiments, an engineered antigen receptor is co-expressed with the cytokine IL-15.

The sequence of the open reading frame encoding the engineered antigen receptor can be obtained from a genomic DNA source, a cDNA source, or can be synthesized (e.g., via PCR), or combinations thereof. Depending upon the size of the genomic DNA and the number of introns, it may be desirable to use cDNA or a combination thereof as it is found that introns stabilize the mRNA. Also, it may be further advantageous to use endogenous or exogenous non-coding regions to stabilize the mRNA.

It is contemplated that the chimeric construct can be introduced into immune cells of any kind as naked DNA or in a suitable vector. Methods of stably transfecting cells by electroporation using naked DNA are known in the art. See, e.g., U.S. Pat. No. 6,410,319. Naked DNA generally refers to the DNA encoding a chimeric receptor contained in a plasmid expression vector in proper orientation for expression.

Alternatively, a viral vector (e.g., a retroviral vector, adenoviral vector, adeno-associated viral vector, or lentiviral vector) or a non-viral method can be used to introduce the chimeric construct into immune cells. Suitable vectors for use in accordance with the method of the present disclosure are non-replicating in the immune cells. A large number of vectors are known that are based on viruses, where the copy number of the virus maintained in the cell is low enough to maintain the viability of the cell, such as, for example, vectors based on HIV, SV40, EBV, HSV, or BPV. Non-viral vectors include plasmids, transposons, nanoparticles, liposome, lipids, metals, or a combination thereof.

In some embodiments, the genetically engineered antigen receptors include recombinant TCRs and/or TCRs cloned from naturally occurring T cells. A “T cell receptor” or “TCR” refers to a molecule that contains a variable a and β chains (also known as TCRα and TCRβ, respectively) or a variable 7 and 6 chains (also known as TCRγ and TCRδ, respectively) and that is capable of specifically binding to an antigen peptide bound to a MHC receptor. In some embodiments, the TCR is in the αβ form.

Typically, TCRs that exist in αβ and 76 forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules. In some embodiments, a TCR also can contain a constant domain, a transmembrane domain and/or a short cytoplasmic tail (see, e.g., Janeway et al, 1997). For example, in some aspects, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction. Unless otherwise stated, the term “TCR” should be understood to encompass functional TCR fragments thereof. The term also encompasses intact or full-length TCRs, including TCRs in the αβ form or 76 form.

Thus, for purposes herein, reference to a TCR includes any TCR or functional fragment, such as an antigen-binding portion of a TCR that binds to a specific antigenic peptide bound in an MHC molecule, i.e. MHC-peptide complex. An “antigen-binding portion” or antigen-binding fragment” of a TCR, which can be used interchangeably, refers to a molecule that contains a portion of the structural domains of a TCR, but that binds the antigen (e.g. MHC-peptide complex) to which the full TCR binds. In some cases, an antigen-binding portion contains the variable domains of a TCR, such as variable a chain and variable p chain of a TCR, sufficient to form a binding site for binding to a specific MHC-peptide complex, such as generally where each chain contains three complementarity determining regions.

In some embodiments, the variable domains of the TCR chains associate to form loops, or complementarity determining regions (CDRs) analogous to immunoglobulins, which confer antigen recognition and determine peptide specificity by forming the binding site of the TCR molecule and determine peptide specificity. Typically, like immunoglobulins, the CDRs are separated by framework regions (FRs) (see, e.g., Jores et al., 1990; Chothia et al., 1988; Lefranc et al., 2003). In some embodiments, CDR3 is the main CDR responsible for recognizing processed antigen, although CDR1 of the alpha chain has also been shown to interact with the N-terminal part of the antigenic peptide, whereas CDR1 of the beta chain interacts with the C-terminal part of the peptide. CDR2 is thought to recognize the MHC molecule. In some embodiments, the variable region of the β-chain can contain a further hypervariability (HV4) region.

In some embodiments, the TCR chains contain a constant domain. For example, like immunoglobulins, the extracellular portion of TCR chains (e.g., a-chain, β-chain) can contain two immunoglobulin domains, a variable domain (e.g., Vₐ or Vp; typically amino acids 1 to 116 based on Kabat numbering Kabat et al., “Sequences of Proteins of Immunological Interest, US Dept. Health and Human Services, Public Health Service National Institutes of Health, 1991, 5^{th} ed.) at the N-terminus, and one constant domain (e.g., a-chain constant domain or Cₐ, typically amino acids 117 to 259 based on Kabat, 1-chain constant domain or Cp, typically amino acids 117 to 295 based on Kabat) adjacent to the cell membrane. For example, in some cases, the extracellular portion of the TCR formed by the two chains contains two membrane-proximal constant domains, and two membrane-distal variable domains containing CDRs. The constant domain of the TCR domain contains short connecting sequences in which a cysteine residue forms a disulfide bond, making a link between the two chains. In some embodiments, a TCR may have an additional cysteine residue in each of the α and β chains such that the TCR contains two disulfide bonds in the constant domains.

In some embodiments, the TCR chains can contain a transmembrane domain. In some embodiments, the transmembrane domain is positively charged. In some cases, the TCR chains contains a cytoplasmic tail. In some cases, the structure allows the TCR to associate with other molecules like CD3. For example, a TCR containing constant domains with a transmembrane region can anchor the protein in the cell membrane and associate with invariant subunits of the CD3 signaling apparatus or complex.

Generally, CD3 is a multi-protein complex that can possess three distinct chains (γ, δ, and ε) in mammals and the ζ-chain. For example, in mammals the complex can contain a CD3γ chain, a CD3δ chain, two CD3ε chains, and a homodimer of CD3δ chains. The CD3γ, CD3δ, and CD3ε chains are highly related cell surface proteins of the immunoglobulin superfamily containing a single immunoglobulin domain. The transmembrane regions of the CD3γ, CD3δ, and CD3ε chains are negatively charged, which is a characteristic that allows these chains to associate with the positively charged T cell receptor chains. The intracellular tails of the CD3γ, CD3δ, and CD3ε chains each contain a single conserved motif known as an immunoreceptor tyrosine-based activation motif or ITAM, whereas each CD3ζ chain has three. Generally, ITAMs are involved in the signaling capacity of the TCR complex. These accessory molecules have negatively charged transmembrane regions and play a role in propagating the signal from the TCR into the cell. The CD3− and ζ-chains, together with the TCR, form what is known as the T cell receptor complex.

In some embodiments, the TCR may be a heterodimer of two chains a and β (or optionally γ and δ) or it may be a single chain TCR construct. In some embodiments, the TCR is a heterodimer containing two separate chains (α and β chains or γ and δ chains) that are linked, such as by a disulfide bond or disulfide bonds. In some embodiments, a TCR for a target antigen (e.g., a cancer antigen) is identified and introduced into the cells. In some embodiments, nucleic acid encoding the TCR can be obtained from a variety of sources, such as by polymerase chain reaction (PCR) amplification of publicly available TCR DNA sequences. In some embodiments, the TCR is obtained from a biological source, such as from cells such as from a T cell (e.g. cytotoxic T cell), T cell hybridomas or other publicly available source. In some embodiments, the T cells can be obtained from in vivo isolated cells. In some embodiments, a high-affinity T cell clone can be isolated from a patient, and the TCR isolated. In some embodiments, the T cells can be a cultured T cell hybridoma or clone. In some embodiments, the TCR clone for a target antigen has been generated in transgenic mice engineered with human immune system genes (e.g., the human leukocyte antigen system, or HLA). See, e.g., tumor antigens (see, e.g., Parkhurst et al., 2009 and Cohen et al., 2005). In some embodiments, phage display is used to isolate TCRs against a target antigen (see, e.g., Varela-Rohena et al., 2008 and Li, 2005). In some embodiments, the TCR or antigen-binding portion thereof can be synthetically generated from knowledge of the sequence of the TCR.

### B. Antigens

The CARs and TCRs of the disclosure target one or more particular antigens. Among the antigens targeted by the genetically engineered antigen receptors are those expressed in the context of a disease, condition, or cell type to be targeted via the adoptive cell therapy. Among the diseases and conditions are proliferative, neoplastic, and malignant diseases and disorders, including cancers and tumors, including hematologic cancers, cancers of the immune system, such as lymphomas, leukemias, and/or myelomas, such as B, T, and myeloid leukemias, lymphomas, and multiple myelomas. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

Among the antigens targeted by the genetically engineered antigen receptors are those expressed in the context of a disease, condition, or cell type to be targeted via the adoptive cell therapy. Among the diseases and conditions are proliferative, neoplastic, and malignant diseases and disorders, including cancers and tumors, including hematologic cancers, cancers of the immune system, such as lymphomas, leukemias, and/or myelomas, such as B, T, and myeloid leukemias, lymphomas, and multiple myelomas. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

Any suitable antigen may be targeted in the present method. The antigen may be associated with certain cancer cells but not associated with non-cancerous cells, in some cases. Exemplary antigens include, but are not limited to, antigenic molecules from infectious agents, auto-/self-antigens, tumor-/cancer-associated antigens, and tumor neoantigens (Linnemann et al., 2015). In particular aspects, the antigens include CD19, EBNA, CD123, HER2, CA-125, TRAIL/DR4, CD20, CD70, CD38, trop2, HLA-G, CD123, CLL1, carcinoembryonic antigen, alphafetoprotein, CD56, AKT, Her3, epithelial tumor antigen, CD319 (CS1), ROR1, folate binding protein, HIV-1 envelope glycoprotein gpl20, HIV-1 envelope glycoprotein gp41, CD5, CD23, CD30, HERV-K, IL-11Ralpha, kappa chain, lambda chain, CSPG4, CD33, CD47, CLL-1, U5snRNP200, CD200, BAFF-R, BCMA, CD99, p53, mutated p53, Ras, mutated ras, c-Myc, cytoplasmic serine/threonine kinases (e.g., A-Raf, B-Raf, and C-Raf, cyclin-dependent kinases), MAGE-A1, MAGE-A2, MAGE-A3, MAGE-A4, MAGE-A6, MAGE-A10, MAGE-A12, MART-1, melanoma-associated antigen, BAGE, DAM-6, -10, GAGE-1, -2, -8, GAGE-3, -4, -5, -6, -7B, NA88-A, MC1R, mda-7, gp75, Gp100, PSA, PSM, Tyrosinase, tyrosinase-related protein, TRP-1, TRP-2, ART-4, CAMEL, CEA, Cyp-B, hTERT, hTRT, iCE, MUC1, MUC2, Phosphoinositide 3-kinases (PI3Ks), TRK receptors, PRAME, P15, RU1, RU2, SART-1, SART-3, Wilms' tumor antigen (WTi), AFP, -catenin/m, Caspase-8/m, CDK-4/m, ELF2M, GnT-V, G250, HAGE, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mbcr-abl, BCR-ABL, interferon regulatory factor 4 (IRF4), ETV6/AML, LDLR/FUT, Pml/RAR, Tumor-associated calcium signal transducer 1 (TACSTD1) TACSTD2, receptor tyrosine kinases (e.g., Epidermal Growth Factor receptor (EGFR) (in particular, EGFRvIII), platelet derived growth factor receptor (PDGFR), vascular endothelial growth factor receptor (VEGFR)), VEGFR2, cytoplasmic tyrosine kinases (e.g., src-family, syk-ZAP70 family), integrin-linked kinase (ILK), signal transducers and activators of transcription STAT3, STATS, and STATE, hypoxia inducible factors (e.g., HIF-1 and HIF-2), Nuclear Factor-Kappa B (NF-B), Notch receptors (e.g., Notchl-4), NY ESO 1, c-Met, mammalian targets of rapamycin (mTOR), WNT, extracellular signal-regulated kinases (ERKs), and their regulatory subunits, PMSA, PR-3, MDM2, Mesothelin, renal cell carcinoma-5T4, SM22-alpha, carbonic anhydrases I (CAI) and IX (CAIX) (also known as G250), STEAD, TEL/AML1, GD2, proteinase3, hTERT, sarcoma translocation breakpoints, EphA2, ML-IAP, EpCAM, ERG (TMPRSS2 ETS fusion gene), NA17, PAX3, ALK, androgen receptor, cyclin B1, polysialic acid, MYCN, RhoC, GD3, fucosyl GM1, mesothelian, PSCA, sLe, PLAC1, GM3, BORIS, Tn, GLoboH, NY-BR-1, RGsS, SAGE, SART3, STn, PAX5, OY-TES1, sperm protein 17, LCK, HMWMAA, AKAP-4, SSX2, XAGE 1, B7H3, legumain, TIE2, Page4, MAD-CT-1, FAP, MAD-CT-2, fos related antigen 1, CBX2, CLDN6, SPANX, TPTE, ACTL8, ANKRD30A, CDKN2A, MAD2L1, CTAG1B, SUNC1, and LRRN1. Examples of sequences for antigens are known in the art, for example, in the GenBank® database: CD19 (Accession No. NG_007275.1), EBNA (Accession No. NG_002392.2), WTi (Accession No. NG_009272.1), CD123 (Accession No. NC_000023.11), NY-ESO (Accession No. NC_000023.11), EGFRvIII (Accession No. NG_007726.3), MUC1 (Accession No. NG_029383.1), HER2 (Accession No. NG_007503.1), CA-125 (Accession No. NG_055257.1), WTi (Accession No. NG_009272.1), Mage-A3 (Accession No. NG_013244.1), Mage-A4 (Accession No. NG_013245.1), Mage-A10 (Accession No. NC_000023.11), TRAIL/DR4 (Accession No. NC_000003.12), and/or CEA (Accession No. NC_000019.10).

Tumor-associated antigens may be derived from prostate, breast, colorectal, lung, pancreatic, renal, mesothelioma, ovarian, liver, brain, bone, stomach, spleen, testicular, cervical, anal, gall bladder, thyroid, or melanoma cancers, as examples. Exemplary tumor-associated antigens or tumor cell-derived antigens include MAGE 1, 3, and MAGE 4 (or other MAGE antigens such as those disclosed in International Patent Publication No. WO 99/40188); PRAME; BAGE; RAGE, Lage (also known as NY ESO 1); SAGE; and HAGE or GAGE.

These non-limiting examples of tumor antigens are expressed in a wide range of tumor types such as melanoma, lung carcinoma, sarcoma, and bladder carcinoma. See, e.g., U.S. Pat. No. 6,544,518. Prostate cancer tumor-associated antigens include, for example, prostate specific membrane antigen (PSMA), prostate-specific antigen (PSA), prostatic acid phosphates, NKX3.1, and six-transmembrane epithelial antigen of the prostate (STEAP).

Other tumor associated antigens include Plu-1, HASH-1, HasH-2, Cripto and Criptin. Additionally, a tumor antigen may be a self-peptide hormone, such as full length gonadotrophin hormone releasing hormone (GnRH), a short 10 amino acid long peptide, useful in the treatment of many cancers.

Antigens may include epitopic regions or epitopic peptides derived from genes mutated in tumor cells or from genes transcribed at different levels in tumor cells compared to normal cells, such as telomerase enzyme, survivin, mesothelin, mutated ras, bcr/abl rearrangement, Her2/neu, mutated or wild-type p53, cytochrome P450 1B1, and abnormally expressed intron sequences such as N-acetylglucosaminyltransferase-V; clonal rearrangements of immunoglobulin genes generating unique idiotypes in myeloma and B-cell lymphomas; tumor antigens that include epitopic regions or epitopic peptides derived from oncoviral processes, such as human papilloma virus proteins E6 and E7; Epstein bar virus protein LMP2; nonmutated oncofetal proteins with a tumor-selective expression, such as carcinoembryonic antigen and alpha-fetoprotein.

In other embodiments, an antigen is obtained or derived from a pathogenic microorganism or from an opportunistic pathogenic microorganism (also called herein an infectious disease microorganism), such as a virus, fungus, parasite, and bacterium. In certain embodiments, antigens derived from such a microorganism include full-length proteins.

Illustrative pathogenic organisms whose antigens are contemplated for use in the method described herein include human immunodeficiency virus (HIV), herpes simplex virus (HSV), respiratory syncytial virus (RSV), cytomegalovirus (CMV), Epstein-Barr virus (EBV), Influenza A, B, and C, vesicular stomatitis virus (VSV), vesicular stomatitis virus (VSV), polyomavirus (e.g., BK virus and JC virus), adenovirus, Staphylococcus species including Methicillin-resistant Staphylococcus aureus (MRSA), and Streptococcus species including Streptococcus pneumoniae. As would be understood by the skilled person, proteins derived from these and other pathogenic microorganisms for use as antigen as described herein and nucleotide sequences encoding the proteins may be identified in publications and in public databases such as GENBANK®, SWISS-PROT®, and TREMBL®.

Antigens derived from human immunodeficiency virus (HIV) include any of the HIV virion structural proteins (e.g., gp120, gp41, p17, p24), protease, reverse transcriptase, or HIV proteins encoded by tat, rev, nef, vif, vpr and vpu.

Antigens derived from herpes simplex virus (e.g., HSV 1 and HSV2) include, but are not limited to, proteins expressed from HSV late genes. The late group of genes predominantly encodes proteins that form the virion particle. Such proteins include the five proteins from (UL) which form the viral capsid: UL6, UL18, UL35, UL38 and the major capsid protein UL19, UL45, and UL27, each of which may be used as an antigen as described herein. Other illustrative HSV proteins contemplated for use as antigens herein include the ICP27 (H1, H2), glycoprotein B (gB) and glycoprotein D (gD) proteins. The HSV genome comprises at least 74 genes, each encoding a protein that could potentially be used as an antigen.

Antigens derived from cytomegalovirus (CMV) include CMV structural proteins, viral antigens expressed during the immediate early and early phases of virus replication, glycoproteins I and III, capsid protein, coat protein, lower matrix protein pp65 (ppUL83), p52 (ppUL44), IE1 and 1E2 (UL123 and UL122), protein products from the cluster of genes from UL128-UL150 (Rykman, et al., 2006), envelope glycoprotein B (gB), gH, gN, and pp150. As would be understood by the skilled person, CMV proteins for use as antigens described herein may be identified in public databases such as GENBANK®, SWISS-PROT®, and TREMBL® (see e.g., Bennekov et al., 2004; Loewendorf et al., 2010; Marschall et al., 2009).

Antigens derived from Epstein-Ban virus (EBV) that are contemplated for use in certain embodiments include EBV lytic proteins gp350 and gp110, EBV proteins produced during latent cycle infection including Epstein-Ban nuclear antigen (EBNA)-1, EBNA-2, EBNA-3A, EBNA-3B, EBNA-3C, EBNA-leader protein (EBNA-LP) and latent membrane proteins (LMP)-1, LMP-2A and LMP-2B (see, e.g., Lockey et al., 2008).

Antigens derived from respiratory syncytial virus (RSV) that are contemplated for use herein include any of the eleven proteins encoded by the RSV genome, or antigenic fragments thereof. NS 1, NS2, N (nucleocapsid protein), M (Matrix protein) SH, G and F (viral coat proteins), M2 (second matrix protein), M2-1 (elongation factor), M2-2 (transcription regulation), RNA polymerase, and phosphoprotein P.

Antigens derived from Vesicular stomatitis virus (VSV) that are contemplated for use include any one of the five major proteins encoded by the VSV genome, and antigenic fragments thereof: large protein (L), glycoprotein (G), nucleoprotein (N), phosphoprotein (P), and matrix protein (M) (see, e.g., Rieder et al., 1999).

Antigens derived from an influenza virus that are contemplated for use in certain embodiments include hemagglutinin (HA), neuraminidase (NA), nucleoprotein (NP), matrix proteins M1 and M2, NS1, NS2 (NEP), PA, PB1, PB1-F2, and PB2.

Exemplary viral antigens also include, but are not limited to, adenovirus polypeptides, alphavirus polypeptides, calicivirus polypeptides (e.g., a calicivirus capsid antigen), coronavirus polypeptides, distemper virus polypeptides, Ebola virus polypeptides, enterovirus polypeptides, flavivirus polypeptides, hepatitis virus (AE) polypeptides (a hepatitis B core or surface antigen, a hepatitis C virus E1 or E2 glycoproteins, core, or non-structural proteins), herpesvirus polypeptides (including a herpes simplex virus or varicella zoster virus glycoprotein), infectious peritonitis virus polypeptides, leukemia virus polypeptides, Marburg virus polypeptides, orthomyxovirus polypeptides, papilloma virus polypeptides, parainfluenza virus polypeptides (e.g., the hemagglutinin and neuraminidase polypeptides), paramyxovirus polypeptides, parvovirus polypeptides, pestivirus polypeptides, picorna virus polypeptides (e.g., a poliovirus capsid polypeptide), pox virus polypeptides (e.g., a vaccinia virus polypeptide), rabies virus polypeptides (e.g., a rabies virus glycoprotein G), reovirus polypeptides, retrovirus polypeptides, and rotavirus polypeptides.

In certain embodiments, the antigen may be bacterial antigens. In certain embodiments, a bacterial antigen of interest may be a secreted polypeptide. In other certain embodiments, bacterial antigens include antigens that have a portion or portions of the polypeptide exposed on the outer cell surface of the bacteria.

Antigens derived from Staphylococcus species including Methicillin-resistant Staphylococcus aureus (MRSA) that are contemplated for use include virulence regulators, such as the Agr system, Sar and Sae, the Arl system, Sar homologues (Rot, MgrA, SarS, SarR, SarT, SarU, SarV, SarX, SarZ and TcaR), the Srr system and TRAP. Other Staphylococcus proteins that may serve as antigens include Clp proteins, HtrA, MsrR, aconitase, CcpA, SvrA, Msa, CfvA and CfvB (see, e.g., Staphylococcus: Molecular Genetics, 2008 Caister Academic Press, Ed. Jodi Lindsay). The genomes for two species of Staphylococcus aureus (N315 and Mu50) have been sequenced and are publicly available, for example at PATRIC (PATRIC: The VBI PathoSystems Resource Integration Center, Snyder et al., 2007). As would be understood by the skilled person, Staphylococcus proteins for use as antigens may also be identified in other public databases such as GenBank®, Swiss-Prot®, and TrEMBL®.

Antigens derived from Streptococcus pneumoniae that are contemplated for use in certain embodiments described herein include pneumolysin, PspA, choline-binding protein A (CbpA), NanA, NanB, SpnHL, PavA, LytA, Pht, and pilin proteins (RrgA; RrgB; RrgC). Antigenic proteins of Streptococcuspneumoniae are also known in the art and may be used as an antigen in some embodiments (see, e.g., Zysk et al., 2000). The complete genome sequence of a virulent strain of Streptococcus pneumoniae has been sequenced and, as would be understood by the skilled person, S. pneumoniae proteins for use herein may also be identified in other public databases such as GENBANK®, SWISS-PROT®, and TREMBL®. Proteins of particular interest for antigens according to the present disclosure include virulence factors and proteins predicted to be exposed at the surface of the pneumococci (see, e.g., Frolet et al., 2010).

Examples of bacterial antigens that may be used as antigens include, but are not limited to, Actinomyces polypeptides, Bacillus polypeptides, Bacteroides polypeptides, Bordetella polypeptides, Bartonella polypeptides, Borrelia polypeptides (e.g., B. burgdorferi OspA), Brucella polypeptides, Campylobacter polypeptides, Capnocytophaga polypeptides, Chlamydia polypeptides, Corynebacterium polypeptides, Coxiella polypeptides, Dermatophilus polypeptides, Enterococcus polypeptides, Ehrlichia polypeptides, Escherichia polypeptides, Francisella polypeptides, Fusobacterium polypeptides, Haemobartonella polypeptides, Haemophilus polypeptides (e.g., H. influenzae type b outer membrane protein), Helicobacter polypeptides, Klebsiella polypeptides, L-form bacteria polypeptides, Leptospira polypeptides, Listeria polypeptides, Mycobacteria polypeptides, Mycoplasma polypeptides, Neisseria polypeptides, Neorickettsia polypeptides, Nocardia polypeptides, Pasteurella polypeptides, Peptococcus polypeptides, Peptostreptococcus polypeptides, Pneumococcus polypeptides (i.e., S. pneumoniae polypeptides) (see description herein), Proteus polypeptides, Pseudomonas polypeptides, Rickettsia polypeptides, Rochalimaea polypeptides, Salmonella polypeptides, Shigella polypeptides, Staphylococcus polypeptides, group A streptococcus polypeptides (e.g., S. pyogenes M proteins), group B streptococcus (S. agalactiae) polypeptides, Treponema polypeptides, and Yersinia polypeptides (e.g., Y pestis F1 and V antigens).

Examples of fungal antigens include, but are not limited to, Absidia polypeptides, Acremonium polypeptides, Alternaria polypeptides, Aspergillus polypeptides, Basidiobolus polypeptides, Bipolaris polypeptides, Blastomyces polypeptides, Candida polypeptides, Coccidioides polypeptides, Conidiobolus polypeptides, Cryptococcus polypeptides, Curvalaria polypeptides, Epidermophyton polypeptides, Exophiala polypeptides, Geotrichum polypeptides, Histoplasma polypeptides, Madurella polypeptides, Malassezia polypeptides, Microsporum polypeptides, Moniliella polypeptides, Mortierella polypeptides, Mucor polypeptides, Paecilomyces polypeptides, Penicillium polypeptides, Phialemonium polypeptides, Phialophora polypeptides, Prototheca polypeptides, Pseudallescheria polypeptides, Pseudomicrodochium polypeptides, Pythium polypeptides, Rhinosporidium polypeptides, Rhizopus polypeptides, Scolecobasidium polypeptides, Sporothrix polypeptides, Stemphylium polypeptides, Trichophyton polypeptides, Trichosporon polypeptides, and Xylohypha polypeptides.

Examples of protozoan parasite antigens include, but are not limited to, Babesia polypeptides, Balantidium polypeptides, Besnoitia polypeptides, Cryptosporidium polypeptides, Eimeria polypeptides, Encephalitozoon polypeptides, Entamoeba polypeptides, Giardia polypeptides, Hammondia polypeptides, Hepatozoon polypeptides, Isospora polypeptides, Leishmania polypeptides, Microsporidia polypeptides, Neospora polypeptides, Nosema polypeptides, Pentatrichomonas polypeptides, Plasmodium polypeptides. Examples of helminth parasite antigens include, but are not limited to, Acanthocheilonema polypeptides, Aelurostrongylus polypeptides, Ancylostoma polypeptides, Angiostrongylus polypeptides, Ascaris polypeptides, Brugia polypeptides, Bunostomum polypeptides, Capillaria polypeptides, Chabertia polypeptides, Cooperia polypeptides, Crenosoma polypeptides, Dictyocaulus polypeptides, Dioctophyme polypeptides, Dipetalonema polypeptides, Diphyllobothrium polypeptides, Diplydium polypeptides, Dirofilaria polypeptides, Dracunculus polypeptides, Enterobius polypeptides, Filaroides polypeptides, Haemonchus polypeptides, Lagochilascaris polypeptides, Loa polypeptides, Mansonella polypeptides, Muellerius polypeptides, Nanophyetus polypeptides, Necator polypeptides, Nematodirus polypeptides, Oesophagostomum polypeptides, Onchocerca polypeptides, Opisthorchis polypeptides, Ostertagia polypeptides, Parafilaria polypeptides, Paragonimus polypeptides, Parascaris polypeptides, Physaloptera polypeptides, Protostrongylus polypeptides, Setaria polypeptides, Spirocerca polypeptides Spirometra polypeptides, Stephanofilaria polypeptides, Strongyloides polypeptides, Strongylus polypeptides, Thelazia polypeptides, Toxascaris polypeptides, Toxocara polypeptides, Trichinella polypeptides, Trichostrongylus polypeptides, Trichuris polypeptides, Uncinaria polypeptides, and Wuchereria polypeptides. (e.g., P. falciparum circumsporozoite (PfCSP)), sporozoite surface protein 2 (PfSSP2), carboxyl terminus of liver state antigen 1 (PfLSA1 c-term), and exported protein 1 (PfExp-1), Pneumocystis polypeptides, Sarcocystis polypeptides, Schistosoma polypeptides, Theileria polypeptides, Toxoplasma polypeptides, and Trypanosoma polypeptides.

Examples of ectoparasite antigens include, but are not limited to, polypeptides (including antigens as well as allergens) from fleas; ticks, including hard ticks and soft ticks; flies, such as midges, mosquitoes, sand flies, black flies, horse flies, horn flies, deer flies, tsetse flies, stable flies, myiasis-causing flies and biting gnats; ants; spiders, lice; mites; and true bugs, such as bed bugs and kissing bugs.

In certain embodiments, the technologies disclosed herein utilizing genetically modify immune cells, such as NK cells, comprise (i) non-viral gene transfer using an electroporation device (e.g., a nucleofector), (ii) CARs that signal through endodomains (e.g., CD28/CD3-ζ, CD137/CD3-ζ, or other combinations), (iii) CARs with variable lengths of extracellular domains connecting the antigen-recognition domain to the cell surface, and, in some cases, (iv) artificial antigen presenting cells (aAPC) derived from K562 to be able to robustly and numerically expand CAR⁺ immune cells (Singh et al., 2008; Singh et al., 2011).

As shown herein, in some embodiments, a cell which comprises one or more engineered antigen receptors (e.g., CARs) has increased expression of said engineered antigen receptor following deactivation, cryopreservation, and thawing relative to an otherwise comparable cell that was not deactivated prior to cryopreservation. In some embodiments, such a cell has higher engineered antigen receptor mediated signaling relative to an appropriate control cell.

### C. Suicide Genes

In particular embodiments, a cell described herein comprises a suicide gene to control its use and allow for termination of a cell therapy at a desired event and/or time. In some embodiments, the suicide gene is employed in transduced cells for the purpose of eliciting death for the transduced cells when needed. The cells of the present disclosure that have been modified to harbor a vector encompassed by the disclosure may comprise one or more suicide genes. In some embodiments, the term “suicide gene” as used herein is defined as a gene which, upon administration of a prodrug or other agent, effects transition of a gene product to a compound which kills its host cell. In other embodiments, a suicide gene encodes a gene product that is, when desired, targeted by an agent (such as an antibody) that targets the suicide gene product.

In some cases, the cell therapy may be subject to utilization of one or more suicide genes of any kind when an individual receiving the cell therapy and/or having received the cell therapy shows one or more symptoms of one or more adverse events, such as cytokine release syndrome, neurotoxicity, anaphylaxis/allergy, and/or on-target/off tumor toxicities (as examples) or is considered at risk for having the one or more symptoms, including imminently. The use of the suicide gene may be part of a planned protocol for a therapy or may be used only upon a recognized need for its use. In some cases the cell therapy is terminated by use of agent(s) that targets the suicide gene or a gene product therefrom because the therapy is no longer required.

In some embodiments, utilization of the suicide gene may be instigated upon onset of at least one adverse event for the individual, and that adverse event may be recognized by any means, including upon routine monitoring that may or may not be continuous from the beginning of the cell therapy. The adverse event(s) may be detected upon examination and/or testing. In cases wherein the individual has cytokine release syndrome (which may also be referred to as cytokine storm), the individual may have elevated inflammatory cytokine(s) (merely as examples: interferon-gamma, granulocyte macrophage colony-stimulating factor, IL-10, IL-6 and TNF-alpha); fever; fatigue; hypotension; hypoxia, tachycardia; nausea; capillary leak; cardiac/renal/hepatic dysfunction; or a combination thereof, for example. In cases wherein the individual has neurotoxicity, the individual may have confusion, delirium, aplasia, and/or seizures. In some cases, the individual is tested for a marker associated with onset and/or severity of cytokine release syndrome, such as C-reactive protein, IL-6, TNF-alpha, and/or ferritin.

Examples of suicide genes include engineered nonsecretable (including membrane bound) tumor necrosis factor (TNF)-alpha mutant polypeptides (see PCT/US19/62009, which is incorporated by reference herein in its entirety), and they may be affected by delivery of an antibody that binds the TNF-alpha mutant. Examples of suicide gene/prodrug combinations that may be used are Herpes Simplex Virus-thymidine kinase (HSV-tk) and ganciclovir, acyclovir, or FIAU; oxidoreductase and cycloheximide; cytosine deaminase and 5-fluorocytosine; thymidine kinase thymidylate kinase (Tdk::Tmk) and AZT; and deoxycytidine kinase and cytosine arabinoside. The E. coli purine nucleoside phosphorylase, a so-called suicide gene that converts the prodrug 6-methylpurine deoxyriboside to toxic purine 6-methylpurine, may be utilized. Other suicide genes include CD20, CD52, inducible caspase 9, purine nucleoside phosphorylase (PNP), Cytochrome p450 enzymes (CYP), Carboxypeptidases (CP), Carboxylesterase (CE), Nitroreductase (NTR), Guanine Ribosyltransferase (XGRTP), Glycosidase enzymes, Methionine-α,γ-lyase (MET), and Thymidine phosphorylase (TP), as examples.

In particular embodiments, vectors that encode a engineered antigen receptor (e.g., CAR and/or TCR), or any vector in a cell (e.g., NK cell) encompassed herein, include one or more suicide genes. The suicide gene may or may not be on the same vector as an engineered antigen receptor. In cases wherein the suicide gene is present on the same vector as the engineered antigen receptor, the suicide gene and the engineered antigen receptor may be separated by an IRES or 2A (e.g., P2A, T2A, etc.) element, for example.

### D. Cytokines

In some embodiments, cells disclosed herein (e.g., NK cells) are engineered to express one or more heterologous cytokines and/or are engineered to upregulate normal expression of one or more heterologous cytokines. The cells may or may not be transduced or transfected for one or more cytokines on the same vector as other genes.

One or more cytokines may be co-expressed from a vector, including as a separate polypeptide from the engineered antigen receptor and/or suicide gene. Interleukin-15 (IL-15), for example, is tissue restricted and only under pathologic conditions is it observed at any level in the serum, or systemically. IL-15 possesses several attributes that are desirable for adoptive therapy. IL-15 is a homeostatic cytokine that induces development and cell proliferation of natural killer cells, promotes the eradication of established tumors via alleviating functional suppression of tumor-resident cells, and inhibits activation-induced cell death (AICD). In addition to IL-15, other cytokines are envisioned. These include, but are not limited to, cytokines, chemokines, and other molecules that contribute to the activation and proliferation of cells used for human application. NK cells expressing IL-15 are capable of continued supportive cytokine signaling, which is useful for their survival post-infusion.

In specific embodiments, the cells express one or more exogenously provided cytokines. As one example, the cytokine is IL-15, IL-12, IL-2, IL-18, IL-21, IL-23, GMCSF, or a combination thereof. The cytokine may be exogenously provided to the cells (e.g., NK cells) because it is expressed from an expression vector within the cell. In an alternative case, an endogenous cytokine in the cell is upregulated upon manipulation of regulation of expression of the endogenous cytokine, such as genetic recombination at the promoter site(s) of the cytokine. In cases wherein the cytokine is provided on an expression construct to the cell, the cytokine may be encoded from the same vector as a suicide gene and/or as the CAR. In some embodiments, the present disclosure concerns deactivation and optional storage of an NK cell, wherein the NK cell comprises one or more CARs, IL-15, and optionally a suicide gene.

### E. Knockout or Knockdown of Endogenous Genes

In some embodiments, the cells of the present disclosure that are deactivated and cryopreserved are modified to have altered expression of certain genes such as glucocorticoid receptor, TGFβ receptor (e.g., TGFβ-RII), and/or CISH. In one embodiment, the immune cells may be modified to express a dominant negative TGFβ receptor II (TGFβRIIDN) which can function as a cytokine sink to deplete endogenous TGFβ.

Cytokine signaling is essential for the normal function of hematopoietic cells. The SOCS family of proteins plays an important role in the negative regulation of cytokine signaling, acting as an intrinsic brake. CIS, a member of the SOCS family of proteins encoded by the CISH gene, has been identified as an important checkpoint molecule in NK cells in mice. Thus, in some embodiments, the present disclosure concerns the knockout of CISH in immune cells to improve cytotoxicity of NK cells and CD8⁺ T cells, for example. This approach may be used alone or in combination with other checkpoint inhibitors to improve anti-tumor activity.

In some embodiments, the altered gene expression is carried out by effecting a disruption in the gene, such as a knock-out, insertion, missense or frameshift mutation, such as biallelic frameshift mutation, deletion of all or part of the gene, e.g., one or more exon or portion therefore, and/or knock-in. For example, the altered gene expression can be effected by sequence-specific or targeted nucleases, including DNA-binding targeted nucleases such as zinc finger nucleases (ZFN) and transcription activator-like effector nucleases (TALENs), and RNA-guided nucleases such as a CRISPR-associated nuclease (Cas), specifically designed to be targeted to the sequence of the gene or a portion thereof.

In some embodiments, the alteration of the expression, activity, and/or function of the gene is carried out by disrupting the gene. In some aspects, the gene is modified so that its expression is reduced by at least at or about 20, 30, or 40%, generally at least at or about 50, 60, 70, 80, 90, or 95% as compared to the expression in the absence of the gene modification or in the absence of the components introduced to effect the modification.

In some embodiments, the alteration is transient or reversible, such that expression of the gene is restored at a later time. In other embodiments, the alteration is not reversible or transient, e.g., is permanent.

In some embodiments, gene alteration is carried out by induction of one or more double-stranded breaks and/or one or more single-stranded breaks in the gene, typically in a targeted manner. In some embodiments, the double-stranded or single-stranded breaks are made by a nuclease, e.g. an endonuclease, such as a gene-targeted nuclease. In some aspects, the breaks are induced in the coding region of the gene, e.g. in an exon. For example, in some embodiments, the induction occurs near the N-terminal portion of the coding region, e.g. in the first exon, in the second exon, or in a subsequent exon.

In some aspects, the double-stranded or single-stranded breaks undergo repair via a cellular repair process, such as by non-homologous end-joining (NHEJ) or homology-directed repair (HDR). In some aspects, the repair process is error-prone and results in disruption of the gene, such as a frameshift mutation, e.g., biallelic frameshift mutation, which can result in complete knockout of the gene. For example, in some aspects, the disruption comprises inducing a deletion, mutation, and/or insertion. In some embodiments, the disruption results in the presence of an early stop codon. In some aspects, the presence of an insertion, deletion, translocation, frameshift mutation, and/or a premature stop codon results in disruption of the expression, activity, and/or function of the gene.

In some embodiments, gene alteration is achieved using antisense techniques, such as by RNA interference (RNAi), short interfering RNA (siRNA), short hairpin (shRNA), and/or ribozymes are used to selectively suppress or repress expression of the gene. siRNA technology is RNAi which employs a double-stranded RNA molecule having a sequence homologous with the nucleotide sequence of mRNA which is transcribed from the gene, and a sequence complementary with the nucleotide sequence. siRNA generally is homologous/complementary with one region of mRNA which is transcribed from the gene, or may be siRNA including a plurality of RNA molecules which are homologous/complementary with different regions. In some aspects, the siRNA is comprised in a polycistronic construct.

In some embodiments, the cells of the immediate disclosure may also encompass gene editing of the cells to remove 1, 2, 3, 4, 5, 6, 7, 8, 9, 10, or more endogenous genes in the cells. In some cases the gene editing occurs in NK cells expressing one or more heterologous antigen receptors, whereas in other cases the gene editing occurs in NK cells that do not express a heterologous antigen receptor but that ultimately will express one or more heterologous antigen receptors, in at least some cases. In particular embodiments, the NK cells that are gene edited are expanded and/or activated NK cells. In particular embodiments, the NK cells that are gene edited are NK cells that are or have previously been deactivated.

In particular cases, one or more endogenous genes of the NK cells are modified, such as disrupted in expression where the expression is reduced in part or in full. In specific cases, one or more genes are knocked down or knocked out using processes of the disclosure. In specific cases, multiple genes are knocked down or knocked out in the same step as processes of the disclosure. The genes that are edited in the NK cells may be of any kind, but in specific embodiments the genes are genes whose gene products inhibit activity and/or proliferation of NK cells. In specific cases the genes that are edited in the NK cells allow the NK cells to work more effectively in a tumor microenvironment. In specific cases, the genes are one or more of NKG2A, SIGLEC-7, LAG3, TIM3, CISH, FOXO1, TGFBR2, TIGIT, CD96, ADORA2, NR3C1, PD1, PDL-1, PDL-2, CD47, SIRPA, SHIP1, ADAM17, RPS6, 4EBP1, CD25, CD40, IL21R, ICAM1, CD95, CD80, CD86, IL10R, TDAG8, CD5, CD7, SLAMF7, CD38, LAG3, TCR, beta2-microglobulin, HLA, CD73, CREB1, CREM, ICER, and CD39. In specific embodiments, the TGFBR2 gene is knocked out or knocked down in the NK cells. 1. ZFPs and ZFNs

In some embodiments, the DNA-targeting molecule includes a DNA-binding protein such as one or more zinc finger protein (ZFP) or transcription activator-like protein (TAL), fused to an effector protein such as an endonuclease. Examples include ZFNs, TALEs, and TALENs.

In some embodiments, the DNA-targeting molecule comprises one or more zinc-finger proteins (ZFPs) or domains thereof that bind to DNA in a sequence-specific manner. A ZFP or domain thereof is a protein or domain within a larger protein that binds DNA in a sequence-specific manner through one or more zinc fingers, regions of amino acid sequence within the binding domain whose structure is stabilized through coordination of a zinc ion. The term zinc finger DNA binding protein is often abbreviated as zinc finger protein or ZFP. Among the ZFPs are artificial ZFP domains targeting specific DNA sequences, typically 9-18 nucleotides long, generated by assembly of individual fingers.

ZFPs include those in which a single finger domain is approximately 30 amino acids in length and contains an alpha helix containing two invariant histidine residues coordinated through zinc with two cysteines of a single beta turn, and having two, three, four, five, or six fingers. Generally, sequence-specificity of a ZFP may be altered by making amino acid substitutions at the four helix positions (−1, 2, 3 and 6) on a zinc finger recognition helix. Thus, in some embodiments, the ZFP or ZFP-containing molecule is non-naturally occurring, e.g., is engineered to bind to a target site of choice.

In some embodiments, the DNA-targeting molecule is or comprises a zinc-finger DNA binding domain fused to a DNA cleavage domain to form a zinc-finger nuclease (ZFN). In some embodiments, fusion proteins comprise the cleavage domain (or cleavage half-domain) from at least one Type IIS restriction enzyme and one or more zinc finger binding domains, which may or may not be engineered. In some embodiments, the cleavage domain is from the Type IIS restriction endonuclease Fok I. Fok I generally catalyzes double-stranded cleavage of DNA, at 9 nucleotides from its recognition site on one strand and 13 nucleotides from its recognition site on the other.

Many gene-specific engineered zinc fingers are available commercially. For example, Sangamo Biosciences (Richmond, CA, USA) has developed a platform (CompoZr) for zinc-finger construction in partnership with Sigma-Aldrich (St. Louis, MO, USA), allowing investigators to bypass zinc-finger construction and validation altogether, and provides specifically targeted zinc fingers for thousands of proteins (Gaj et al., Trends in Biotechnology, 2013, 31(7), 397-405). In some embodiments, commercially available zinc fingers are used or are custom designed. (See, for example, Sigma-Aldrich catalog numbers CSTZFND, CSTZFN, CTil-lKT, and PZD0020).

### 2. TALs, TALEs and TALENs

In some embodiments, the DNA-targeting molecule comprises a naturally occurring or engineered (non-naturally occurring) transcription activator-like protein (TAL) DNA binding domain, such as in a transcription activator-like protein effector (TALE) protein, See, e.g., U.S. Patent Publication No. 2011/0301073, incorporated by reference in its entirety herein.

A TALE DNA binding domain or TALE is a polypeptide comprising one or more TALE repeat domains/units. The repeat domains are involved in binding of the TALE to its cognate target DNA sequence. A single “repeat unit” (also referred to as a “repeat”) is typically 33-35 amino acids in length and exhibits at least some sequence homology with other TALE repeat sequences within a naturally occurring TALE protein. Each TALE repeat unit includes 1 or 2 DNA-binding residues making up the Repeat Variable Di-residue (RVD), typically at positions 12 and/or 13 of the repeat. The natural (canonical) code for DNA recognition of these TALEs has been determined such that an HD sequence at positions 12 and 13 leads to a binding to cytosine (C), NG binds to T, NI to A, NN binds to G or A, and NO binds to T and non-canonical (atypical) RVDs are also known. In some embodiments, TALEs may be targeted to any gene by design of TAL arrays with specificity to the target DNA sequence. The target sequence generally begins with a thymidine.

In some embodiments, the molecule is a DNA binding endonuclease, such as a TALE nuclease (TALEN). In some aspects the TALEN is a fusion protein comprising a DNA-binding domain derived from a TALE and a nuclease catalytic domain to cleave a nucleic acid target sequence.

In some embodiments, the TALEN recognizes and cleaves the target sequence in the gene. In some aspects, cleavage of the DNA results in double-stranded breaks. In some aspects the breaks stimulate the rate of homologous recombination or non-homologous end joining (NHEJ). Generally, NHEJ is an imperfect repair process that often results in changes to the DNA sequence at the site of the cleavage. In some aspects, repair mechanisms involve rejoining of what remains of the two DNA ends through direct re-ligation or via the so-called microhomology-mediated end joining. In some embodiments, repair via NHEJ results in small insertions or deletions and can be used to disrupt and thereby repress the gene. In some embodiments, the modification may be a substitution, deletion, or addition of at least one nucleotide. In some aspects, cells in which a cleavage-induced mutagenesis event, i.e. a mutagenesis event consecutive to an NHEJ event, has occurred can be identified and/or selected by well-known methods in the art.

In some embodiments, TALE repeats are assembled to specifically target a gene. (Gaj et al., 2013). A library of TALENs targeting 18,740 human protein-coding genes has been constructed (Kim et al., 2013). Custom-designed TALE arrays are commercially available through Cellectis Bioresearch (Paris, France), Transposagen Biopharmaceuticals (Lexington, KY, USA), and Life Technologies (Grand Island, NY, USA). Specifically, TALENs that target CD38 are commercially available (See Gencopoeia, catalog numbers HTN222870-1, HTN222870-2, and HTN222870−3). Exemplary molecules are described, e.g., in U.S. Patent Publication Nos. US 2014/0120622, and 2013/0315884.

In some embodiments the TALENs are introduced as trans genes encoded by one or more plasmid vectors. In some aspects, the plasmid vector can contain a selection marker which provides for identification and/or selection of cells which received said vector.

3. RGENs (CRISPR/Cas systems)

In some embodiments, the alteration is carried out using one or more DNA-binding nucleic acids, such as alteration via an RNA-guided endonuclease (RGEN). For example, the alteration can be carried out using clustered regularly interspaced short palindromic repeats (CRISPR) and CRISPR-associated (Cas) proteins. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus. Methods of utilizing a CRISPR system are well known in the art, but are described briefly herein.

The CRISPR/Cas nuclease or CRISPR/Cas nuclease system can include a non-coding RNA molecule (guide) RNA, which sequence-specifically binds to DNA, and a Cas protein (e.g., Cas9), with nuclease functionality (e.g., two nuclease domains). One or more elements of a CRISPR system can derive from a type I, type II, or type III CRISPR system, e.g., derived from a particular organism comprising an endogenous CRISPR system, such as Streptococcus pyogenes.

In some aspects, a Cas nuclease and gRNA (including a fusion of crRNA specific for the target sequence and fixed tracrRNA) are introduced into the cell. In general, target sites at the 5′ end of the gRNA target the Cas nuclease to the target site, e.g., the gene, using complementary base pairing. The target site may be selected based on its location immediately 5′ of a protospacer adjacent motif (PAM) sequence, such as typically NGG, or NAG. In this respect, the gRNA is targeted to the desired sequence by modifying the first 20, 19, 18, 17, 16, 15, 14, 14, 12, 11, or 10 nucleotides of the guide RNA to correspond to the target DNA sequence. In general, a CRISPR system is characterized by elements that promote the formation of a CRISPR complex at the site of a target sequence. Typically, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex.

The CRISPR system can induce double stranded breaks (DSBs) at the target site, followed by disruptions or alterations as discussed herein. In other embodiments, Cas9 variants, deemed “nickases,” are used to nick a single strand at the target site. Paired nickases can be used, e.g., to improve specificity, each directed by a pair of different gRNAs targeting sequences such that upon introduction of the nicks simultaneously, a 5′ overhang is introduced. In other embodiments, catalytically inactive Cas9 is fused to a heterologous effector domain such as a transcriptional repressor or activator, to affect gene expression.

The target sequence may comprise any polynucleotide, such as DNA or RNA polynucleotides. The target sequence may be located in the nucleus or cytoplasm of the cell, such as within an organelle of the cell. Generally, a sequence or template that may be used for recombination into the targeted locus comprising the target sequences is referred to as an “editing template” or “editing polynucleotide” or “editing sequence”. In some aspects, an exogenous template polynucleotide may be referred to as an editing template. In some aspects, the recombination is homologous recombination.

Typically, in the context of an endogenous CRISPR system, formation of the CRISPR complex (comprising the guide sequence hybridized to the target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. The tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of the CRISPR complex, such as by hybridization along at least a portion of the tracr sequence to all or a portion of a tracr mate sequence that is operably linked to the guide sequence. The tracr sequence has sufficient complementarity to a tracr mate sequence to hybridize and participate in formation of the CRISPR complex, such as at least 50%, 60%, 70%, 80%, 90%, 95% or 99% of sequence complementarity along the length of the tracr mate sequence when optimally aligned.

One or more vectors driving expression of one or more elements of the CRISPR system can be introduced into the cell such that expression of the elements of the CRISPR system direct formation of the CRISPR complex at one or more target sites. Components can also be delivered to cells as proteins and/or RNA. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. The vector may comprise one or more insertion sites, such as a restriction endonuclease recognition sequence (also referred to as a “cloning site”). In some embodiments, one or more insertion sites are located upstream and/or downstream of one or more sequence elements of one or more vectors. When multiple different guide sequences are used, a single expression construct may be used to target CRISPR activity to multiple different, corresponding target sequences within a cell.

A vector may comprise a regulatory element operably linked to an enzyme-coding sequence encoding the CRISPR enzyme, such as a Cas protein. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes are known; for example, the amino acid sequence of S. pyogenes Cas9 protein may be found in the SwissProt database under accession number Q99ZW2.

The CRISPR enzyme can be Cas9 (e.g., from S. pyogenes or S. pneumonia). The CRISPR enzyme can direct cleavage of one or both strands at the location of a target sequence, such as within the target sequence and/or within the complement of the target sequence. The vector can encode a CRISPR enzyme that is mutated with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). In some embodiments, a Cas9 nickase may be used in combination with guide sequence(s), e.g., two guide sequences, which target respectively sense and antisense strands of the DNA target. This combination allows both strands to be nicked and used to induce NHEJ or HDR.

In some embodiments, an enzyme coding sequence encoding the CRISPR enzyme is codon optimized for expression in particular cells, such as eukaryotic cells. The eukaryotic cells may be those of or derived from a particular organism, such as a mammal, including but not limited to human, mouse, rat, rabbit, dog, sheep, or non-human primate. In general, codon optimization refers to a process of modifying a nucleic acid sequence for enhanced expression in the host cells of interest by replacing at least one codon of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

In general, a guide sequence is any polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more.

Exemplary gRNA sequences for NR3CS (glucocorticoid receptor) include Ex3 NR3C1 sG1 5-TGC TGT TGA GGA GCT GGA-3 (SEQ ID NO:1) and Ex3 NR3C1 sG2 5-AGC ACA CCA GGC AGA GTT-3 (SEQ ID NO:2). Exemplary gRNA sequences for TGF-beta receptor 2 include EX3 TGFBR2 sG1 5-CGG CTG AGG AGC GGA AGA-3 (SEQ ID NO:3) and EX3 TGFBR2 sG2 5-TGG-AGG-TGA-GCA-ATC-CCC-3 (SEQ ID NO:4). The T7 promoter, target sequence, and overlap sequence may have the sequence TTAATACGACTCACTATAGG (SEQ ID NO:5)+target sequence+gttttagagctagaaatagc (SEQ ID NO:6).

Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), Clustal W, Clustal X, BLAT, Novoalign (Novocraft Technologies, ELAND (Illumina, San Diego, Calif.), SOAP (available at soap.genomics.org.cn), and Maq (available at maq.sourceforge.net).

The CRISPR enzyme may be part of a fusion protein comprising one or more heterologous protein domains. A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (RP), chloramphenicol acetyltransferase (CAT) beta galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A CRISPR enzyme may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4A DNA binding domain fusions, and herpes simplex virus (HSV) BP16 protein fusions. Additional domains that may form part of a fusion protein comprising a CRISPR enzyme are described in US 20110059502, incorporated herein by reference.

### D. Methods of Delivery

The cells encompassed herein may harbor a recombinant vector. In some embodiments, cells may be transformed, transfected, and/or transduced either before deactivation and cryopreservation, or following thawing after deactivation and cryopreservation. One of skill in the art would be well-equipped to construct a vector through standard recombinant techniques (see, for example, Sambrook et al., 2001 and Ausubel et al., 1996, both incorporated herein by reference) for the expression of the antigen receptors of the present disclosure. Vectors include but are not limited to, plasmids, cosmids, viruses (bacteriophage, animal viruses, and plant viruses), and artificial chromosomes (e.g., YACs), such as retroviral vectors (e.g. derived from Moloney murine leukemia virus vectors (MoMLV), MSCV, SFFV, MPSV, SNV etc), lentiviral vectors (e.g. derived from HIV-1, HIV-2, SIV, BIV, FIV etc.), adenoviral (Ad) vectors including replication competent, replication deficient and gutless forms thereof, adeno-associated viral (AAV) vectors, simian virus 40 (SV-40) vectors, bovine papilloma virus vectors, Epstein-Barr virus vectors, herpes virus vectors, vaccinia virus vectors, Harvey murine sarcoma virus vectors, murine mammary tumor virus vectors, Rous sarcoma virus vectors, parvovirus vectors, polio virus vectors, vesicular stomatitis virus vectors, maraba virus vectors and group B adenovirus enadenotucirev vectors.

### 1. Viral Vectors

Viral vectors encoding an antigen receptor may be provided in certain aspects of the present disclosure. In generating recombinant viral vectors, non-essential genes are typically replaced with a gene or coding sequence for a heterologous (or non-native) protein. A viral vector is a kind of expression construct that utilizes viral sequences to introduce nucleic acid and possibly proteins into a cell. The ability of certain viruses to infect cells or enter cells via receptor mediated-endocytosis, and to integrate into host cell genomes and express viral genes stably and efficiently have made them attractive candidates for the transfer of foreign nucleic acids into cells (e.g., mammalian cells). Non-limiting examples of virus vectors that may be used to deliver a nucleic acid of certain aspects of the present invention are described below.

Lentiviruses are complex retroviruses, which, in addition to the common retroviral genes gag, pol, and env, contain other genes with regulatory or structural function. Lentiviral vectors are well known in the art (see, for example, U.S. Pat. Nos. 6,013,516 and 5,994,136).

Recombinant lentiviral vectors are capable of infecting non-dividing cells and can be used for both in vivo and ex vivo gene transfer and expression of nucleic acid sequences. For example, recombinant lentivirus capable of infecting a non-dividing cell—wherein a suitable host cell is transfected with two or more vectors carrying the packaging functions, namely gag, pol and env, as well as rev and tat—is described in U.S. Pat. No. 5,994,136, incorporated herein by reference.

### 2. Regulatory Elements

Expression cassettes included in vectors useful in the present disclosure in particular contain (in a 5′-to-3′ direction) a eukaryotic transcriptional promoter operably linked to a protein-coding sequence, splice signals including intervening sequences, and a transcriptional termination/polyadenylation sequence. The promoters and enhancers that control the transcription of protein encoding genes in eukaryotic cells are composed of multiple genetic elements. The cellular machinery is able to gather and integrate the regulatory information conveyed by each element, allowing different genes to evolve distinct, often complex patterns of transcriptional regulation. A promoter used in the context of the present disclosure includes constitutive, inducible, and tissue-specific promoters.

### 3. Promoter/Enhancers

The expression constructs provided herein comprise a promoter to drive expression of the antigen receptor. A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30110 bp—upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the Plactamase (penicillinase), lactose and tryptophan (trp-) promoter systems. In addition to producing nucleic acid sequences of promoters and enhancers synthetically, sequences may be produced using recombinant cloning and/or nucleic acid amplification technology, including PCR™, in connection with the compositions disclosed herein. Furthermore, it is contemplated that the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 1989, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally, any promoter/enhancer combination (as per, for example, the Eukaryotic Promoter Data Base EPDB, through world wide web at epd.isb-sib.ch/) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

Non-limiting examples of promoters include early or late viral promoters, such as, SV40 early or late promoters, cytomegalovirus (CMV) immediate early promoters, Rous Sarcoma Virus (RSV) early promoters; eukaryotic cell promoters, such as, e.g., beta actin promoter, GAPDH promoter, metallothionein promoter; and concatenated response element promoters, such as cyclic AMP response element promoters (cre), serum response element promoter (sre), phorbol ester promoter (TPA) and response element promoters (tre) near a minimal TATA box. It is also possible to use human growth hormone promoter sequences (e.g., the human growth hormone minimal promoter described at Genbank, accession no. X05244, nucleotide 283-341) or a mouse mammary tumor promoter (available from the ATCC, Cat. No. ATCC 45007). In certain embodiments, the promoter is CMV IE, dectin-1, dectin-2, human CD11c, F4/80, SM22, RSV, SV40, Ad MLP, beta-actin, MHC class I or MHC class II promoter, however any other promoter that is useful to drive expression of the therapeutic gene is applicable to the practice of the present disclosure.

In certain aspects, methods of the disclosure also concern enhancer sequences, i.e., nucleic acid sequences that increase a promoter's activity and that have the potential to act in cis, and regardless of their orientation, even over relatively long distances (up to several kilobases away from the target promoter). However, enhancer function is not necessarily restricted to such long distances as they may also function in close proximity to a given promoter.

### 4. Initiation Signals and Linked Expression

A specific initiation signal also may be used in the expression constructs provided in the present disclosure for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments, the use of internal ribosome entry sites (IRES) elements are used to create multigene, or polycistronic, messages. IRES elements are able to bypass the ribosome scanning model of 5′ methylated Cap dependent translation and begin translation at internal sites. IRES elements from two members of the picornavirus family (polio and encephalomyocarditis) have been described, as well an IRES from a mammalian message. IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

Additionally, certain 2A sequence elements could be used to create linked- or co-expression of genes in the constructs provided in the present disclosure. For example, cleavage sequences could be used to co-express genes by linking open reading frames to form a single cistron. An exemplary cleavage sequence is the F2A (Foot-and-mouth disease virus 2A) or a “2A-like” sequence (e.g., Thosea asigna virus 2A; T2A).

### 5. Origins of Replication

In order to propagate a vector in a host cell, it may contain one or more origins of replication sites (often termed “ori”), for example, a nucleic acid sequence corresponding to oriP of EBV as described above or a genetically engineered oriP with a similar or elevated function in programming, which is a specific nucleic acid sequence at which replication is initiated. Alternatively a replication origin of other extra-chromosomally replicating virus as described above or an autonomously replicating sequence (ARS) can be employed.

### 6. Selection and Screenable Markers

In some embodiments, cells containing a construct of the present disclosure may be identified in vitro or in vivo by including a marker in the expression vector. Such markers would confer an identifiable change to the cell permitting easy identification of cells containing the expression vector. Generally, a selection marker is one that confers a property that allows for selection. A positive selection marker is one in which the presence of the marker allows for its selection, while a negative selection marker is one in which its presence prevents its selection. An example of a positive selection marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selection markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes as negative selection markers such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selection and screenable markers are well known to one of skill in the art.

### 7. Other Methods of Nucleic Acid Delivery

In addition to viral delivery of the nucleic acids encoding transgenes as described herein, the following are additional methods of recombinant gene delivery to a given host cell and are thus considered in the present disclosure.

Introduction of a nucleic acid, such as DNA or RNA, into the immune cells of the current disclosure may use any suitable methods for nucleic acid delivery for transformation of a cell, as described herein or as would be known to one of ordinary skill in the art. Such methods include, but are not limited to, direct delivery of DNA such as by ex vivo transfection, by injection, including microinjection); by electroporation; by calcium phosphate precipitation; by using DEAE-dextran followed by polyethylene glycol; by direct sonic loading; by liposome mediated transfection and receptor-mediated transfection; by microprojectile bombardment; by agitation with silicon carbide fibers; by Agrobacterium-mediated transformation; by desiccation/inhibition-mediated DNA uptake, and any combination of such methods. Through the application of techniques such as these, organelle(s), cell(s), tissue(s) or organism(s) may be stably or transiently transformed.

### IV. Methods of Treatment

In some embodiments, the present disclosure provides methods for immunotherapy comprising administering an effective amount of the deactivated and cryopreserved cells of the present disclosure following thawing and reactivation. In some embodiments, a medical disease or disorder is treated by transfer of a cell population previously deactivated and cryopreserved, such as an NK cell population that elicits an immune response.

In some embodiments, methods comprising technologies of the present disclosure comprise administering to a subject a therapeutically effective amount of an optionally loaded, optionally pre-activated, optionally engineered, and optionally expanded cells (e.g., NK cells), that have been deactivated, optionally cryopreserved, and reactivated (e.g., spontaneous reactivation following removal of a deactivating agent), thereby treating or preventing the disease in the subject, including reducing the risk of, reducing the severity of, and/or delaying the onset of the disease. In certain embodiments of the present disclosure, cancer or infection is treated by transfer of a composition comprising cells described herein. In at least some cases, wherein the cells are NK cells, because of their release of pro-inflammatory cytokines, NK cells may reverse the anti-inflammatory tumor microenvironment and increase adaptive immune responses by promoting differentiation, activation, and/or recruitment of accessory immune cell to sites of malignancy.

Cells may be thawed using thawing methods and conditions that are suitable for a clinical product. The cells following thawing may or may not be washed to remove substantially all of the cryopreservation medium and/or deactivating agent prior to administration of the cells to an individual. The cells following thawing may be diluted without washing and infused. The cells may be delivered to an individual substantially immediately upon thawing, or there may be a delay before delivery on the order of 1-24 hours or 1 or more days, for example, including if the cells were washed before infusion. The delivery may be by any route and may depend on the medical condition being treated. The delivery may be local or systemic. With respect to infusion volumes of doses of cells being delivered, the infusion volume may or may not depend on whether or not the subject has already received a dose of cells. For example, a first dose of cells may or may not be greater in volume than a subsequent dose. Multiple infusion volumes may be of the same volume. In some embodiments, the infusion volume of the cells is 1, 5, 10, 15, 20, 30, 40, 50, 60, 70, 75, 80, 90, 100, 125, 150, 175, 200, 225, 250, 275, or 300 or more mL. The liquid in which the cells are suspended for infusion may be of any kind. In specific embodiments, the liquid is PLASMA-LYTE A or a similar solution. The liquid in which the cells are suspended for infusion may or may not comprise human serum albumin, for example. Albumin is a cryoprotectant that can also be used as a non-serum alternative, so it has dual effects. Prior to delivery to an individual in need thereof, the thawed cells may be tested for one or more characteristic, such as the presence of microbes, for example by contamination; viability; cell count, and so forth. In specific embodiments, the cells for infusion are comprised in a solution that comprises one or more other therapeutic agents than the cells themselves.

In certain embodiments of the present disclosure, cancer or infection is treated by transfer of a deactivated and cryopreserved, and subsequently thawed and reactivated population, such as an NK cell population that elicits an immune response. Provided herein are methods for treating or delaying progression of cancer in an individual comprising administering to the individual an effective amount an antigen-specific cell therapy. The present methods may be applied for the treatment of immune disorders, solid cancers, hematologic cancers, viral infections, and regenerative medicine.

Tumors for which the present treatment methods are useful include any malignant cell type, such as those found in a solid tumor or a hematological tumor. Exemplary solid tumors can include, but are not limited to, a tumor of an organ selected from the group consisting of pancreas, colon, cecum, stomach, brain, head, neck, ovary, kidney, larynx, sarcoma, lung, bladder, melanoma, prostate, and breast. Exemplary hematological tumors include tumors of the bone marrow, T or B cell malignancies, leukemias, lymphomas, blastomas, myelomas, and the like. Further examples of cancers that may be treated using the methods provided herein include, but are not limited to, lung cancer (including small-cell lung cancer, non-small cell lung cancer, adenocarcinoma of the lung, and squamous carcinoma of the lung), cancer of the peritoneum, gastric or stomach cancer (including gastrointestinal cancer and gastrointestinal stromal cancer), pancreatic cancer, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, vulval cancer, thyroid cancer, various types of head and neck cancer, and melanoma.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; lentigo malignant melanoma; acral lentiginous melanomas; nodular melanomas; malignant melanoma in giant pigmented nevus; epithelioid cell melanoma; blue nevus, malignant; sarcoma; fibrosarcoma; fibrous histiocytoma, malignant; myxosarcoma; liposarcoma; renal cell carcinoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; hairy cell leukemia; chronic lymphocytic leukemia (CLL); acute lymphoblastic leukemia (ALL); acute myeloid leukemia (AML); and chronic myeloblastic leukemia.

Particular embodiments concern methods of treatment of leukemia. Leukemia is a cancer of the blood or bone marrow and is characterized by an abnormal proliferation (production by multiplication) of blood cells, usually white blood cells (leukocytes) but can involve red blood cells (erythroleukemia). It is part of the broad group of diseases called hematological neoplasms. Leukemia is a broad term covering a spectrum of diseases. Leukemia is clinically and pathologically split into its acute and chronic forms.

Acute leukemia is characterized by the rapid proliferation of immature blood cells. This crowding makes the bone marrow unable to produce healthy blood cells. Acute forms of leukemia can occur in children and young adults. In fact, it is a more common cause of death for children in the U.S. than any other type of malignant disease. Immediate treatment is required in acute leukemia due to the rapid progression and accumulation of the malignant cells, which then spill over into the bloodstream and spread to other organs of the body. Central nervous system (CNS) involvement is uncommon, although the disease can occasionally cause cranial nerve palsies. Chronic leukemia is distinguished by the excessive buildup of relatively mature, but still abnormal, blood cells. Typically taking months to years to progress, the cells are produced at a much higher rate than normal cells, resulting in many abnormal white blood cells in the blood. Chronic leukemia mostly occurs in older people, but can theoretically occur in any age group. Whereas acute leukemia must be treated immediately, chronic forms are sometimes monitored for some time before treatment to ensure maximum effectiveness of therapy.

Furthermore, the diseases are classified into lymphocytic or lymphoblastic, which indicate that the cancerous change took place in a type of marrow cell that normally goes on to form lymphocytes, and myelogenous or myeloid, which indicate that the cancerous change took place in a type of marrow cell that normally goes on to form red cells, some types of white cells, and platelets (see lymphoid cells vs. myeloid cells).

Acute lymphocytic leukemia (also known as acute lymphoblastic leukemia, or ALL) is the most common type of leukemia in young children. This disease also affects adults, especially those aged 65 and older. Chronic lymphocytic leukemia (CLL) most often affects adults over the age of 55. It sometimes occurs in younger adults, but it almost never affects children. Acute myelogenous leukemia (also known as acute myeloid leukemia, or AML) occurs more commonly in adults than in children. This type of leukemia was previously called “acute nonlymphocytic leukemia.” Chronic myelogenous leukemia (CML) occurs mainly in adults. A very small number of children also develop this disease.

Lymphoma is a type of cancer that originates in lymphocytes (a type of white blood cell in the vertebrate immune system). There are many types of lymphoma. According to the U.S. National Institutes of Health, lymphomas account for about five percent of all cases of cancer in the United States, and Hodgkin's lymphoma in particular accounts for less than one percent of all cases of cancer in the United States. Because the lymphatic system is part of the body's immune system, patients with a weakened immune system, such as from HIV infection or from certain drugs or medication, also have a higher incidence of lymphoma.

In certain embodiments of the disclosure, compositions comprising the deactivated, cryopreserved, thawed, and reactivated cells (e.g., NK cells) are delivered to an individual in need thereof, such as an individual that has cancer or an infection. In at least some cases, the cells can enhance the individual's immune system to attack the respective cancer or pathogenic cells. In some cases, the individual is provided with one or more doses of the compositions comprising cells of the present disclosure. In cases where the individual is provided with two or more doses of the cells of the present disclosure, the duration between the administrations should be sufficient to allow time for propagation in the individual, and in specific embodiments the duration between doses is 1, 2, 3, 4, 5, 6, 7, or more days.

In some embodiments, the source of NK cells that are pre-activated (optionally) expanded (optionally), deactivated and cryopreserved, and subsequently thawed and reactivated, may be of any kind, but in specific embodiments the cells are obtained from a bank of umbilical cord blood, peripheral blood, human embryonic stem cells, or induced pluripotent stem cells, for example. Suitable doses for a therapeutic effect would be at least 10⁵ or between about 10⁵ and about 10¹² cells per dose, for example, preferably in a series of dosing cycles. An exemplary dosing regimen consists of four one-week dosing cycles of escalating doses, starting at least at about 10⁵ cells on Day 0, for example increasing incrementally up to a target dose of about 10¹² cells within several weeks of initiating an intra-patient dose escalation scheme. Suitable modes of administration include intravenous, subcutaneous, intracavitary (for example by reservoir-access device), intraperitoneal, and direct injection into a tumor mass.

In some embodiments, the compositions comprising the cells generated according to the present methods have many potential uses, including experimental and therapeutic uses. In particular, it is envisaged that such cell populations are useful in suppressing undesirable or inappropriate immune responses. In such methods, a small number of cells (e.g., NK cells) are removed from a patient and then manipulated and expanded ex vivo before deactivating and storing them for a period of time, then thawing and reactivating the cells and reinfusing them into the patient. Examples of diseases which may be treated in this way are autoimmune diseases and conditions in which suppressed immune activity is desirable, e.g., for allo-transplantation tolerance. A therapeutic method could comprise obtaining NK cells from a mammal; expanding the NK cells ex vivo in accordance with the methods of the present methods as described herein; deactivating and cryopreserving the cells through methods described herein; thawing and reactivating the cells through methods described herein, and administering the compositions comprising the NK cells to the mammal to be treated.

A pharmaceutical composition of the present disclosure comprising cells as described herein can be used alone or in combination with other well-established agents useful for treating cancer. Whether delivered alone or in combination with other agents, the pharmaceutical composition of the present disclosure can be delivered via various routes and to various sites in a mammalian, particularly human, body to achieve a particular effect. One skilled in the art will recognize that, although more than one route can be used for administration, a particular route can provide a more immediate and more effective reaction than another route. For example, intradermal delivery may be advantageously used over inhalation for the treatment of melanoma. Local or systemic delivery can be accomplished by administration comprising application or instillation of the formulation into body cavities, inhalation or insufflation of an aerosol, or by parenteral introduction, comprising intramuscular, intravenous, intraportal, intrahepatic, peritoneal, subcutaneous, or intradermal administration.

Certain embodiments of the present disclosure provide methods for treating or preventing an immune-mediated disorder. In one embodiment, the subject has an autoimmune disease. Non-limiting examples of autoimmune diseases include: alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac spate-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn's disease, discoid lupus, essential mixed cryoglobulinemia, fibromyalgia-fibromyositis, glomerulonephritis, Graves' disease, Guillain-Barre, Hashimoto's thyroiditis, idiopathic pulmonary fibrosis, idiopathic thrombocytopenia purpura (ITP), IgA neuropathy, juvenile arthritis, lichen planus, lupus erthematosus, Meniere's disease, mixed connective tissue disease, multiple sclerosis, type 1 or immune-mediated diabetes mellitus, myasthenia gravis, nephrotic syndrome (such as minimal change disease, focal glomerulosclerosis, or mebranous nephropathy), pemphigus vulgaris, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynaud's phenomenon, Reiter's syndrome, Rheumatoid arthritis, sarcoidosis, scleroderma, Sjogren's syndrome, stiff-man syndrome, systemic lupus erythematosus, lupus erythematosus, ulcerative colitis, uveitis, vasculitides (such as polyarteritis nodosa, takayasu arteritis, temporal arteritis/giant cell arteritis, or dermatitis herpetiformis vasculitis), vitiligo, and Wegener's granulomatosis. Thus, some examples of an autoimmune disease that can be treated using the methods disclosed herein include, but are not limited to, multiple sclerosis, rheumatoid arthritis, systemic lupus erythematosis, type I diabetes mellitus, Crohn's disease; ulcerative colitis, myasthenia gravis, glomerulonephritis, ankylosing spondylitis, vasculitis, or psoriasis. The subject can also have an allergic disorder such as Asthma.

In yet another embodiment, the subject is the recipient of a transplanted organ or stem cells and immune cells are used to prevent and/or treat rejection. In particular embodiments, the subject has or is at risk of developing graft versus host disease. GVHD is a possible complication of any transplant that uses or contains stem cells from either a related or an unrelated donor. There are two kinds of GVHD, acute and chronic. Acute GVHD appears within the first three months following transplantation. Signs of acute GVHD include a reddish skin rash involving small areas of the body initially (chest, back, arms, legs) and that may spread and become more severe encompassing >80% of the body, with peeling or blistering skin. Acute GVHD can also affect the gastrointestinal (GI) tract, in which case nausea and vomiting (upper GI GVHD) and/or abdominal cramping and diarrhea (lower GI GVHD) are present. Yellowing of the skin and eyes (jaundice) indicates that acute GVHD has affected the liver. Chronic GVHD is ranked based on its severity: stage/grade 1 is mild; stage/grade 4 is severe. Chronic GVHD develops three months or later following transplantation. The symptoms of chronic GVHD are similar to those of acute GVHD, but in addition, chronic GVHD may also affect the mucous glands in the eyes, salivary glands in the mouth, and glands that lubricate the stomach lining and intestines. Any of the populations of immune cells disclosed herein can be utilized. Examples of a transplanted organ include a solid organ transplant, such as kidney, liver, skin, pancreas, lung and/or heart, or a cellular transplant such as islets, hepatocytes, myoblasts, bone marrow, or hematopoietic or other stem cells. The transplant can be a composite transplant, such as tissues of the face. Immune cells can be administered prior to transplantation, concurrently with transplantation, or following transplantation. In some embodiments, the immune cells are administered prior to the transplant, such as at least 1 hour, at least 12 hours, at least 1 day, at least 2 days, at least 3 days, at least 4 days, at least 5 days, at least 6 days, at least 1 week, at least 2 weeks, at least 3 weeks, at least 4 weeks, or at least 1 month prior to the transplant. In one specific, non-limiting example, administration of the therapeutically effective amount of immune cells occurs 3-5 days prior to transplantation.

In some embodiments, cells as described herein administered to a patient that is receiving a transplant can be sensitized with antigens specific to the transplanted material prior to administration. According to this embodiment, the transplant recipient will have a decreased immune/inflammatory response to the transplanted material and, as such, the likelihood of rejection of the transplanted tissue is minimized. Similarly, with regard to the treatment of graft versus host disease, the cells (e.g., NK cells) can be sensitized with antigens specific to the host. According to this embodiment, the recipient will have a decreased immune/inflammatory response to self-antigens.

Administration of compositions comprising cells as described herein can be utilized whenever immunosuppression or inhibition of inflammation is desired, for example, at the first sign or symptoms of a disease or inflammation. These may be general, such as pain, edema, elevated temperature, or may be specific signs or symptoms related to dysfunction of affected organ(s). For example, in renal transplant rejection there may be an elevated serum creatinine level, whereas in GVHD, there may be a rash, and in asthma, there may be shortness of breath and wheezing.

In a further embodiment, administration of a therapeutically effective amount of compositions comprising cells as described herein to a subject treats or inhibits inflammation in the subject. Thus, the method includes administering a therapeutically effective amount of compositions comprising cells as described herein (e.g., NK cells) to the subject to inhibit the inflammatory process. Examples of inflammatory disorders include, but are not limited to, asthma, encephalitis, inflammatory bowel disease, chronic obstructive pulmonary disease (COPD), allergic disorders, septic shock, pulmonary fibrosis, undifferentiated spondyloarthropathy, undifferentiated arthropathy, arthritis, inflammatory osteolysis, and chronic inflammation resulting from chronic viral or bacterial infections. The methods disclosed herein can also be used to treat allergic disorders.

In some embodiments, the subject can be administered nonmyeloablative lymphodepleting chemotherapy prior to immune cell therapy comprising cells as described herein. The nonmyeloablative lymphodepleting chemotherapy can be any suitable such therapy, which can be administered by any suitable route. The nonmyeloablative lymphodepleting chemotherapy can comprise, for example, the administration of cyclophosphamide and fludarabine, particularly if the cancer is melanoma, which can be metastatic. An exemplary route of administering cyclophosphamide and fludarabine is intravenously. Likewise, any suitable dose of cyclophosphamide and fludarabine can be administered and is the most common regimen as lymphodepleting chemotherapy before the administration of CAR-T cells or CAR-NK cells. In particular aspects, around 60 mg/kg of cyclophosphamide is administered for two days after which around 25 mg/m² fludarabine is administered for five days.

In certain embodiments, a growth factor that promotes the growth and activation of the immune cells is administered to the subject either concomitantly with the immune cells or subsequently to the immune cells. The immune cell growth factor can be any suitable growth factor that promotes the growth and activation of the immune cells. Examples of suitable immune cell growth factors include interleukin (IL)-2, IL-7, IL-15, and IL-12, which can be used alone or in various combinations, such as IL-2 and IL-7, IL-2 and IL-15, IL-7 and IL-15, IL-2, IL-7 and IL-15, IL-12 and IL-7, IL-12 and IL-15, or IL-12 and IL2.

Therapeutically effective doses of immune cells can be administered by a number of routes, including parenteral administration, for example, intravenous, intraperitoneal, intramuscular, intrasternal, or intraarticular injection, or infusion.

The therapeutically effective dose of immune cells for use in adoptive cell therapy is that amount that achieves a desired effect in a subject being treated. For instance, this can be the dose of immune cells necessary to inhibit advancement, or to cause regression of an autoimmune or alloimmune disease, or which is capable of relieving symptoms caused by an autoimmune disease, such as pain and inflammation. It can be the amount necessary to relieve symptoms associated with inflammation, such as pain, edema and elevated temperature. It can also be the amount necessary to diminish or prevent rejection of a transplanted organ.

The immune cell population can be administered in treatment regimens consistent with the disease, for example a single or a few doses over one to several days to ameliorate a disease state or periodic doses over an extended time to inhibit disease progression and prevent disease recurrence. The precise dose to be employed in the formulation will also depend on the route of administration, and the seriousness of the disease or disorder, and should be decided according to the judgment of the practitioner and each patient's circumstances. The therapeutically effective dose of immune cells will be dependent on the subject being treated, the severity and type of the affliction, and the manner of administration. In some embodiments, doses that could be used in the treatment of human subjects range from at least 3.8×10⁴, at least 3.8×10⁵, at least 3.8×10⁶, at least 3.8×10⁷, at least 3.8×10⁸, at least 3.8×10⁹, or at least 3.8×10¹⁰ immune cells/m². In a certain embodiment, the dose used in the treatment of human subjects ranges from about 3.8×10⁹ to about 3.8×10¹⁰ immune cells/m². In additional embodiments, a therapeutically effective amount of immune cells can vary from about 5×10⁶ cells per kg body weight to about 7.5×10⁸ cells per kg body weight, such as about 2×10⁷ cells to about 5×10⁸ cells per kg body weight, or about 5×10⁷ cells to about 2×10⁶ cells per kg body weight. In some embodiments, doses that could be used in the treatment of human subjects range from at least about, or about 4×10⁶, 8×10⁶, 4×10⁷, 8×10⁷, 4×10⁸, 8×10⁸, 4×10⁹, 8×10⁹, 4×10¹⁰, or 8×10¹⁰ cells per patient per dose. In some embodiments, a dose that could be used in the treatment of human subjects is at least about, or about 4×10⁶ cells per patient per dose. In some embodiments, a dose that could be used in the treatment of human subjects is at least about, or about 8×10⁶ cells per patient per dose. In some embodiments, a patient may be treated with multiple doses. In some embodiments, a patient may be treated with different doses at different times, respectively. In some embodiments, a patient may be treated with the same dose at different times, respectively. In some embodiments, a patient may be treated with different doses at certain times and treated with the same dose at other times, respectively. The exact amount of immune cells is readily determined by one of skill in the art based on the age, weight, sex, and physiological condition of the subject. Effective doses can be extrapolated from dose-response curves derived from in vitro or animal model test systems.

The immune cells may be administered in combination with one or more other therapeutic agents for the treatment of the immune-mediated disorder. Combination therapies can include, but are not limited to, one or more anti-microbial agents (for example, antibiotics, anti-viral agents and anti-fungal agents), anti-tumor agents (for example, fluorouracil, methotrexate, paclitaxel, fludarabine, etoposide, doxorubicin, or vincristine), immune-depleting agents (for example, fludarabine, etoposide, doxorubicin, or vincristine), immunosuppressive agents (for example, azathioprine, or glucocorticoids, such as dexamethasone or prednisone), anti-inflammatory agents (for example, glucocorticoids such as hydrocortisone, dexamethasone or prednisone, or non-steroidal anti-inflammatory agents such as acetylsalicylic acid, ibuprofen or naproxen sodium), cytokines (for example, interleukin-10 or transforming growth factor-beta), hormones (for example, estrogen), or a vaccine. In addition, immunosuppressive or tolerogenic agents including but not limited to calcineurin inhibitors (e.g., cyclosporin and tacrolimus); mTOR inhibitors (e.g., Rapamycin); mycophenolate mofetil, antibodies (e.g., recognizing CD3, CD4, CD40, CD154, CD45, IVIG, or B cells); chemotherapeutic agents (e.g., Methotrexate, Treosulfan, Busulfan); irradiation; or chemokines, interleukins or their inhibitors (e.g., BAFF, IL-2, anti-IL-2R, IL-4, JAK kinase inhibitors) can be administered. Such additional pharmaceutical agents can be administered before, during, or after administration of the immune cells, depending on the desired effect. This administration of the cells and the agent can be by the same route or by different routes, and either at the same site or at a different site.

In certain embodiments, the compositions comprising cells as described herein are administered in combination with a second therapeutic agent. For example, the second therapeutic agent may comprise T cells, an immunomodulatory agent, a monoclonal antibody, a chemotherapeutic agent, hormone(s), drugs of any kind, surgery, radiation, etc. In non-limiting examples, the immunomodulatory agent is lenalidomide, the monoclonal antibody is rituximab, ofatumumab, or lumiliximab, and the chemotherapeutic agent is fludarabine or cyclophosphamide.

A composition of the present disclosure can be provided in unit dosage form wherein each dosage unit, e.g., an injection, contains a predetermined amount of the composition, alone or in appropriate combination with other active agents. The term unit dosage form as used herein refers to physically discrete units suitable as unitary dosages for human and animal subjects, each unit containing a predetermined quantity of the composition of the present disclosure, alone or in combination with other active agents, calculated in an amount sufficient to produce the desired effect, in association with a pharmaceutically acceptable diluent, carrier, or vehicle, where appropriate. The specifications for the unit dosage forms of the present disclosure depend on the particular pharmacodynamics associated with the pharmaceutical composition in the particular subject.

Desirably an effective amount or sufficient number of the cells of the present disclosure is present in the composition and introduced into the subject such that long-term, specific, anti-tumor responses are established to reduce the size of a tumor or eliminate tumor growth or regrowth than would otherwise result in the absence of such treatment. Desirably, the amount of the cells of the present disclosure (e.g., NK cells that had been deactivated, cryopreserved, thawed, and reactivated) reintroduced into the subject causes a 10%, 20%, 30%, 40%, 50%, 60%, 70%, 80%, 90%, 95%, 98%, or 100% decrease in tumor size when compared to otherwise same conditions wherein the NK cells are not deactivated prior to cryopreservation as described herein.

### V. Pharmaceutical Compositions

Also provided herein are pharmaceutical compositions and formulations comprising cells (e.g., NK cells) that were subject to deactivation and cryopreservation, and a pharmaceutically acceptable carrier.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The pharmaceutical compositions may comprise different types of carriers depending on whether it is to be administered in solid, liquid or aerosol form, and whether it need to be sterile for such routes of administration as injection. The presently disclosed compositions can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The compositions comprising the cells of the present disclosure may be formulated into a composition in a free base, neutral or salt form. Pharmaceutically acceptable salts, where appropriate include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

Further in accordance with the present disclosure, the compositions of the present disclosure suitable for administration are provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a the composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present disclosure, the composition is combined with the carrier in any convenient and practical manner, i.e., by solution, suspension, emulsification, admixture, encapsulation, absorption and the like. Such procedures are routine for those skilled in the art.

In a specific embodiment of the present disclosure, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc.

In further embodiments, the present disclosure may concern the use of a pharmaceutical lipid vehicle compositions that include compositions comprising cells of the present disclosure and optionally an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds that contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a nanoparticle or in a lipid vehicle. For example, the compositions comprising the NK cells and antibodies may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present disclosure administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

The therapeutic compositions comprising the cells of the present disclosure may be administered intravenously, intramuscularly, subcutaneously, topically, orally, transdermally, intraperitoneally, intraorbitally, by implantation, by inhalation, intrathecally, intraventricularly, or intranasally. In some embodiments, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice, in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain embodiments, the effective dose of the pharmaceutical composition is one which can provide a blood level of about 1 μM to 150 μM. In another embodiment, the effective dose provides a blood level of about 4 μM to 100 μM; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 M or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

### A. Alimentary Compositions and Formulations

In particular embodiments of the present disclosure, the compositions comprising the cells of the present disclosure are formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain embodiments, the active compounds may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792, 451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof, a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration the compositions of the present disclosure may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

Additional formulations that are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

### B. Parenteral Compositions and Formulations

In further embodiments, compositions may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intravenously, intrathecally, intraventricularly, intra-tumorally, subcutaneously, or intraperitoneally U.S. Pat. Nos. 6,613,308; 5,466,468; 5,543,158; 5,641,515; and 5,399,363 (each specifically incorporated herein by reference in its entirety).

In some embodiments, solutions comprising cells of the immediate disclosure and active compounds may provide said active compounds as free base or pharmacologically acceptable salts that may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and either added hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the cells of the present disclosure and any optional active compounds in the required amount in the appropriate solvent with various of the other ingredients enumerated above, as required, followed by suitable filtration methods and/or sterilization methods (e.g., filtered sterilization). Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

### C. Miscellaneous Pharmaceutical Compositions and Formulations

In other particular embodiments of the disclosure, the active compound compositions comprising the cells of the immediate disclosure may be formulated for administration via various miscellaneous routes, for example, topical (i.e., transdermal) administration, mucosal administration (intranasal, vaginal, etc.) and/or inhalation.

Pharmaceutical compositions for topical administration may include the active compound formulated for a medicated application such as an ointment, paste, cream or powder. Ointments include all oleaginous, adsorption, emulsion and water-soluble based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and laurocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present invention may also comprise the use of a “patch”. For example, the patch may supply one or more active substances at a predetermined rate and in a continuous manner over a fixed period of time.

In certain embodiments, the pharmaceutical compositions may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroethylene support matrix is described in U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present invention for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

### VI. Combination Therapies

In certain embodiments, the compositions and methods of the present embodiments involve cells of the immediate disclosure (e.g., NK cells previously deactivated and cryopreserved, and subsequently thawed and reactivated) in combination with at least one additional therapy. The additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, or a combination of the foregoing. The additional therapy may be in the form of adjuvant or neoadjuvant therapy.

In some embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is chemotherapy. In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is therapy targeting PBK/AKT/mTOR pathway, HSP90 inhibitor, tubulin inhibitor, apoptosis inhibitor, and/or chemopreventative agent. The additional therapy may be one or more of the chemotherapeutic agents known in the art.

In some embodiments, a patient may be treated with the compositions and methods of the immediate disclosure (e.g., NK cells previously expanded, deactivated, cryopreserved, and subsequently thawed) followed by chemotherapy. In some embodiments, a patient may be treated with the compositions and methods of the immediate disclosure simultaneously with chemotherapy. In some embodiments, a patient may be treated with chemotherapy followed by the compositions and methods of the immediate disclosure. In some embodiments, a patient may be treated with cyclophosphamide and/or fludarabine followed by the compositions and methods of the immediate disclosure. In some embodiments, a patient may be treated with cyclophosphamide at a dose level of about 300 mg/m² and fludarabine at a dose level of about 30 mg/m² followed by treatment with compositions and methods of the immediate disclosure. In some embodiments, a patient may be first treated each day for three days with cyclophosphamide at a daily dose level of about 300 mg/m² and fludarabine at a daily dose level of about 30 mg/m², and 48 hours after the last doses of cyclophosphamide and fludarabine, the patient is treated with compositions and methods of the immediate disclosure. In some embodiments, the patient treated in accordance with aspects of the present disclosure is treated for renal cell carcinoma, glioblastoma, mesothelioma, and/or osteosarcoma.

An immune cell therapy (e.g., comprising and/or in addition to the cells of the immediate disclosure) may be administered before, during, after, or in various combinations relative to an additional cancer therapy, such as immune checkpoint therapy. The administrations may be in intervals ranging from concurrently to minutes to days to weeks. In embodiments where the immune cell therapy is provided to a patient separately from an additional therapeutic agent, one would generally ensure that a significant period of time did not expire between the time of each delivery, such that the two compounds would still be able to exert an advantageously combined effect on the patient. In such instances, it is contemplated that one may provide a patient with the antibody therapy and the anti-cancer therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

Various combinations may be employed. For the example below an immune cell therapy is “A” and an anti-cancer therapy is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

### A. Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammalI and calicheamicin omegaI1); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine,plicomycin, gemcitabien, navelbine, farnesyl-protein tansferase inhibitors, transplatinum, and pharmaceutically acceptable salts, acids, or derivatives of any of the above.

### B. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

### C. Immunotherapy

The skilled artisan will understand that additional immunotherapies may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells

Antibody-drug conjugates have emerged as a breakthrough approach to the development of cancer therapeutics. Cancer is one of the leading causes of deaths in the world. Antibody-drug conjugates (ADCs) comprise monoclonal antibodies (MAbs) that are covalently linked to cell-killing drugs. This approach combines the high specificity of MAbs against their antigen targets with highly potent cytotoxic drugs, resulting in “armed” MAbs that deliver the payload (drug) to tumor cells with enriched levels of the antigen. Targeted delivery of the drug also minimizes its exposure in normal tissues, resulting in decreased toxicity and improved therapeutic index. The approval of two ADC drugs, ADCETRIS® (brentuximab vedotin) in 2011 and KADCYLA® (trastuzumab emtansine or T-DM1) in 2013 by FDA validated the approach. There are currently more than 30 ADC drug candidates in various stages of clinical trials for cancer treatment (Leal et al., 2014). As antibody engineering and linker-payload optimization are becoming more and more mature, the discovery and development of new ADCs are increasingly dependent on the identification and validation of new targets that are suitable to this approach and the generation of targeting MAbs. Two criteria for ADC targets are upregulated/high levels of expression in tumor cells and robust internalization.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD19, CD20, CA-125, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al., 1998); cytokine therapy, e.g., interferons α, β, and γ, IL-1, GM-CSF, and TNF (Bukowski et al., 1998; Davidson et al., 1998; Hellstrand et al., 1998); gene therapy, e.g., TNF, IL-1, IL-2, and p53 (Qin et al., 1998; Austin-Ward and Villaseca, 1998; U.S. Pat. Nos. 5,830,880 and 5,846,945); and monoclonal antibodies, e.g., anti-CD20, anti-ganglioside GM2, and anti-p185 (Hollander, 2012; Hanibuchi et al., 1998; U.S. Pat. No. 5,824,311). It is contemplated that one or more anti-cancer therapies may be employed with the antibody therapies described herein.

In some embodiments, the immunotherapy may be an immune checkpoint inhibitor. Immune checkpoints either turn up a signal (e.g., co-stimulatory molecules) or turn down a signal. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies (e.g., International Patent Publication WO2015016718; Pardoll, Nat Rev Cancer, 12(4): 252-64, 2012; both incorporated herein by reference). Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present disclosure. For example it is known that lambrolizumab is also known under the alternative and equivalent names MK-3475 and pembrolizumab.

In some embodiments, the PD-1 binding antagonist is a molecule that inhibits the binding of PD-1 to its ligand binding partners. In a specific aspect, the PD-1 ligand binding partners are PDL1 and/or PDL2. In another embodiment, a PDL1 binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 axis antagonists for use in the methods provided herein are known in the art such as described in U.S. Patent Application No. US20140294898, US2014022021, and US20110008369, all incorporated herein by reference.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIVO©, is an anti-PD-1 antibody described in WO2006/121168. Pembrolizumab, also known as MK-3475, Merck 3475, lambrolizumab, KEYTRUDA©, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PDL2-Fc fusion soluble receptor described in WO2010/027827 and WO2011/066342.

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to CD80 and CD86, also called B7-1 and B7-2 respectively, on antigen-presenting cells. CTLA4 transmits an inhibitory signal to T cells, whereas CD28 transmits a stimulatory signal. Intracellular CTLA4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al. (1998) Proc Natl Acad Sci USA 95(17): 10067-10071; Camacho et al. (2004) J Clin Oncology 22(145): Abstract No. 2505 (antibody CP-675206); and Mokyr et al. (1998) Cancer Res 58:5301-5304 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001014424, WO2000037504, and U.S. Pat. No. 8,017,114; all incorporated herein by reference.

An exemplary anti-CTLA-4 antibody is ipilimumab (also known as 10D1, MDX-010, MDX-101, and Yervoy®) or antigen binding fragments and variants thereof (see, e.g., WO 01/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 90%, 95%, or 99% variable region identity with ipilimumab).

Other molecules for modulating CTLA-4 include CTLA-4 ligands and receptors such as described in U.S. Pat. Nos. 5,844,905, 5,885,796 and International Patent Application Nos. WO1995001994 and WO1998042752; all incorporated herein by reference, and immunoadhesins such as described in U.S. Pat. No. 8,329,867, incorporated herein by reference.

### D. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### E. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### VII. Articles of Manufacture or Kits

An article of manufacture or a kit is provided comprising cryopreservation medium or components thereof and optionally immune cells. The article of manufacture or kit can further comprise a package insert comprising instructions for using the cryopreservation and/or immune cells to treat or delay progression of cancer in an individual or to enhance immune function of an individual having cancer. Any of the cryopreservation media components and optionally antigen-specific immune cells described herein may be included in the article of manufacture or kits. Suitable containers include, for example, bottles, vials, bags and syringes. The container may be formed from a variety of materials such as glass, plastic (such as polyvinyl chloride or polyolefin), or metal alloy (such as stainless steel or hastelloy). In some embodiments, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some embodiments, the article of manufacture further includes one or more of another agent (e.g., a chemotherapeutic agent, and anti-neoplastic agent). Suitable containers for the one or more agent include, for example, bottles, vials, bags and syringes.

In some embodiments, a kit that can include, for example, cells of the immediate disclosure (e.g., NK cells), optionally one or more media and components for the production of said cells, one or more deactivating agents, and so forth are provided. In some embodiments, formulations may comprise a cocktail of factors, including in a form suitable for combining with NK cells. The reagent system or any kit component may be packaged either in aqueous media or in lyophilized form, where appropriate. The container means of the kits will generally include at least one vial, test tube, flask, bottle, syringe or other container means, into which a component may be placed, and preferably, suitably aliquoted. Where there is more than one component in the kit, the kit also will generally contain a second, third or other additional container into which the additional components may be separately placed. However, various combinations of components may be comprised in a vial. The components of the kit may be provided as dried powder(s). When reagents and/or components are provided as a dry powder, the powder can be reconstituted by the addition of a suitable solvent. It is envisioned that the solvent may also be provided in another container means. The kits also will typically include a means for containing the kit component(s) in close confinement for commercial sale. Such containers may include injection or blow molded plastic containers into which the desired vials are retained. The kit can also include instructions for use, such as in printed or electronic format, such as digital format.

In specific embodiments, the kit may comprise one or more cytokines, including at least IL-12, IL-15, IL-18, and/or IL-2, including in particular concentrations as described elsewhere herein. The kit may comprise any type of media, any component of a cryopreservation media, as described elsewhere herein. The kit may comprise cord blood (including pooled cord blood), antigen presenting cells of any kind, beads for depletion of particular NK cells (as described herein), vectors encoding one or more proteins as described herein, NK cells, deactivating agents, antibodies or reagents to generate antibodies, etc.

Individual components may also be provided in a kit in concentrated amounts; in some embodiments, a component is provided individually in the same concentration as it would be in a solution with other components. Concentrations of components may be provided as 1×, 2×, 5×, 10×, or 20× or more.

### VIII Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Isolation, Pre-Activation, Expansion, Deactivation, and Cryopreservation of NK Cells

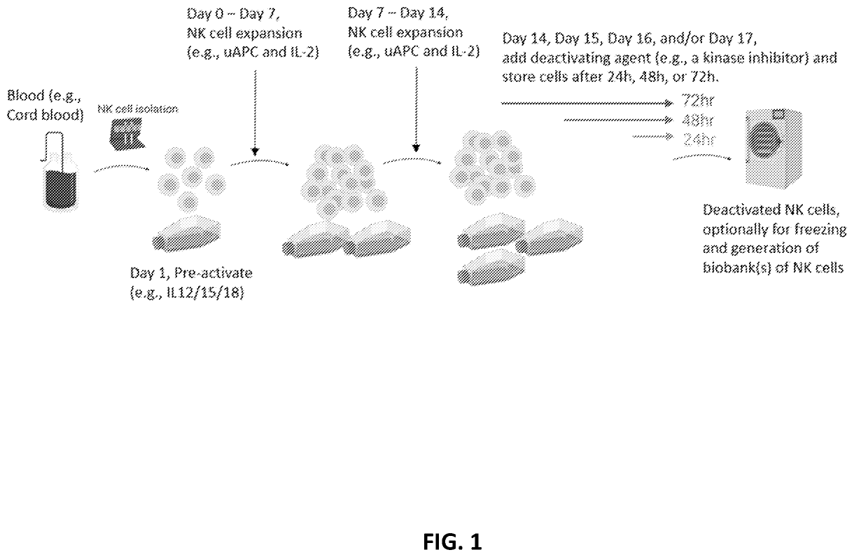

As shown schematically in FIG. 1, NK cells were isolated, pre-activated, expanded, deactivated, and cryopreserved as described herein. Cells were isolated from human CB, pre-activated with a cytokine cocktail comprising IL-12, IL-15, and IL-18, expanded in the presence of uAPCs and IL-2 for 14-17 days, and on day 14, 15, 16, or 17 were treated with a deactivating agent, specifically the TK inhibitor Dasatinib for either 24, 48, or 72 hours (or not treated with Dasatinib as control).

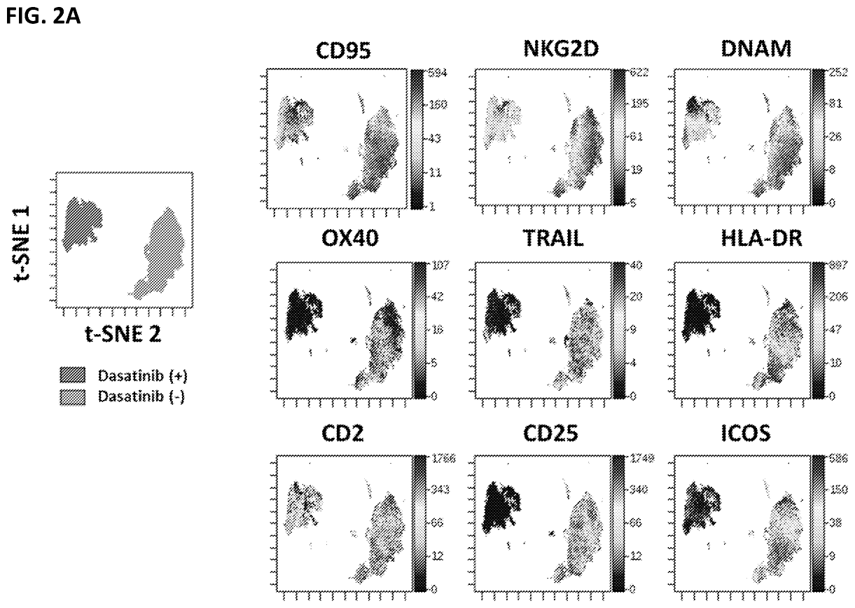

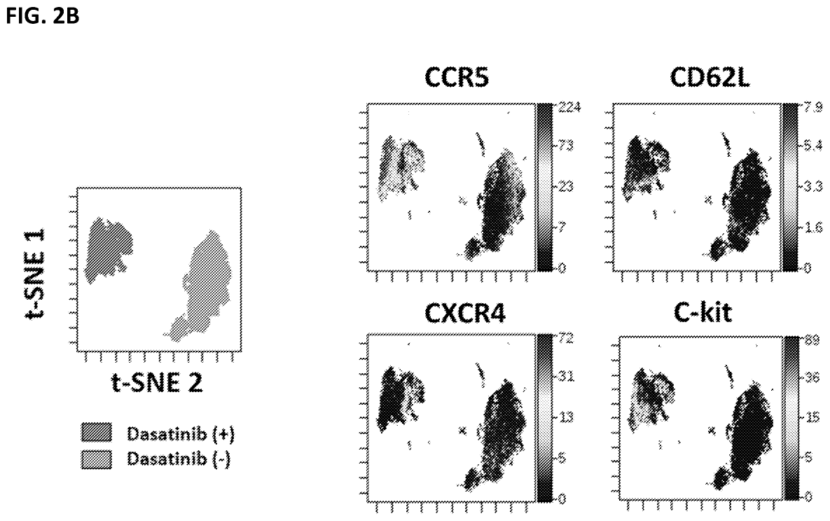

As shown in FIG. 2, addition of Dasatinib prior to cryopreservation switched the NK cell phenotype to a less activated state when analyzed using mass cytometry. On the left side of FIGS. 2A and 2B are t-distributed stochastic neighbor embedding (t-SNE) plots showing a clear delineation in phenotypes between activated NK cells treated with Dasatinib, and those not treated with Dasatinib. As shown in FIG. 2A, the relative expression of activation and cytotoxicity markers such a CD95, NKG2D, DNAM, OX-40, TRAIL, HLA-DR, CD2, CD25, and ICOS, displayed increased expression in NK cells not treated with Dasatinib when compared to NK cells treated with Dasatinib. As shown in FIG. 2B, the relative expression of activation and cytotoxicity markers such a CCR5, CD62L, CXCR4, and C-kit displayed decreased expression in NK cells not treated with Dasatinib when compared to NK cells treated with Dasatinib. These results showed that treatment of cells (e.g., NK cells) with a deactivating agent (e.g., Dasatinib) effectively deactivated the cells prior to cryopreservation. This deactivation was determined to be transient and dependent upon the presence of the deactivating agent, wherein removal of the deactivating agent (e.g., Dasatinib) resulted in reactivation of the cells.

### Example 2—Deactivation Pre-Cryopreservation Improves CAR-NK Cell Viability and Transgene Expression

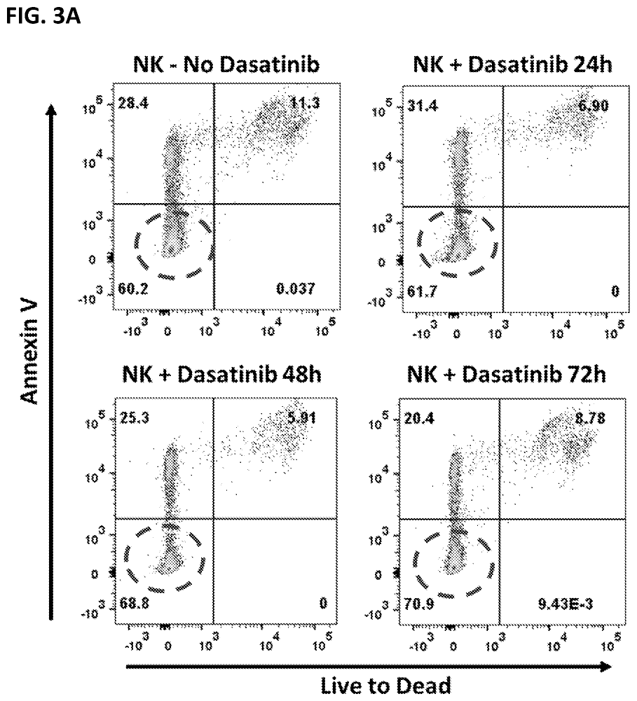

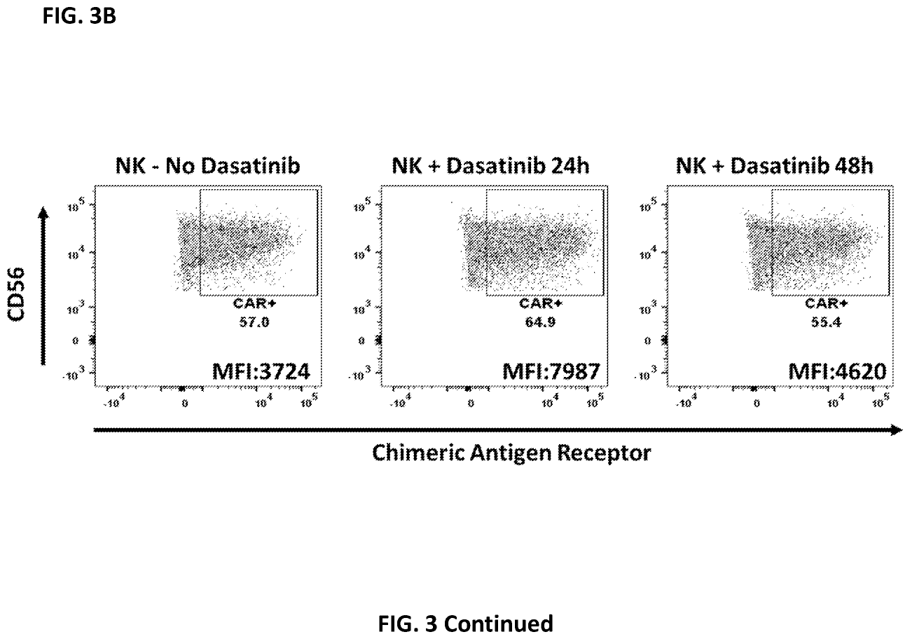

As shown in FIGS. 3A-3B, treatment with Dasatinib pre-cryopreservation improved NK cell viability post thaw without negatively affecting CAR expression. CAR-NK cells were isolated, pre-activated, expanded, and treated with Dasatinib for 24, 48, or 72 hours prior to cryopreservation as described in Example 1 (or not treated with Dasatinib as control). Immediately prior to cryopreservation, CAR-NK cells were washed to remove the deactivating agent. Cells were thawed and analyzed by flow cytometry to determine the number of living cells, NK cells treated with Dasatinib for 24, 48 or 72 hours (h) prior to cryopreservation displayed comparable and/or improved viability relative to NK cells not treated with Dasatinib prior to cryopreservation. Highlighted and quantified in the bottom left quadrant of each graph displayed in FIG. 3A are the living cell percentages, which were 60.2% for no Dasatinib, 61.7% for 24 h Dasatinib treatment, 68.8% for 48 h Dasatinib treatment, and 70.9% for 72 h Dasatinib treatment. As shown in FIG. 3B, the NK cells treated with Dasatinib for 24 or 48 hours prior to cryopreservation displayed comparable and/or improved chimeric antigen receptor (CAR) expression levels relative to NK cells not treated with Dasatinib prior to cryopreservation. The percentage of cells that are CAR positive and the Mean Fluorescence Intensity (MFI) is displayed for each test condition, which were 57% CAR+ for no Dasatinib with MFI of 3724, 64.9% CAR+ for 24 h Dasatinib with MFI of 7987, and 55.4% CAR+ for 48 h Dasatinib with MFI of 4620. These results showed that treatment of cells (e.g., CAR-NK cells) with a deactivating agent (e.g., Dasatinib) improved cell resistance to the damages induced during cryopreservation. Additionally, these results showed that cells comprising a transgene (e.g., a CAR) treated with a deactivating agent prior to cryopreservation continued to express the transgene upon thawing and spontaneous reactivation (e.g., by removal of the deactivating agent).

### Example 3—Deactivation Pre-Cryopreservation Improves NK Cell Viability and NK Cell Cytotoxicity In-Vitro

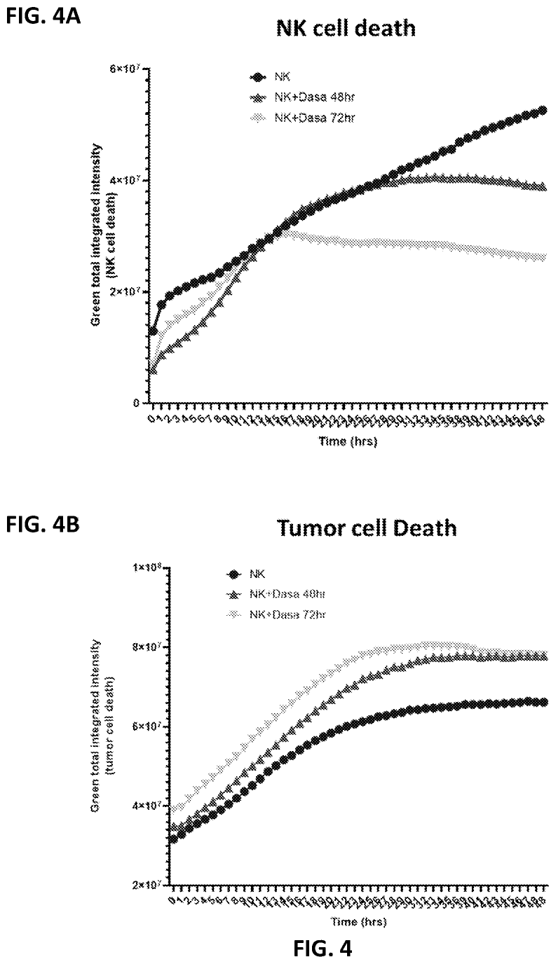

As shown in FIGS. 4A-4B, addition of Dasatinib for 48 or 72 hours pre-cryopreservation improved NK cell viability and anti-tumor cytotoxicity post thaw. FIG. 4A is a graph showing NK cell death following cryopreservation and subsequent thawing. Cytotox green dye was used to measure NK cell death, and green total integrated intensity was used as a surrogate for NK cell death (Y axis) over time (X axis). This data showed that treatment of NK cells with Dasatinib for 48 or 72 hours prior to cryopreservation enhanced NK cell viability post-thaw compared to NK cells that were not deactivated prior to cryopreservation. FIG. 4B is a graph depicting the death of tumor cells (e.g., Raji tumor cells) when cocultured with cryopreserved and thawed NK cells at a 1:1 effector target ratio. Cytotox green dye was used to measure tumor cell death, and green total integrated intensity was used as a surrogate for tumor cell death (Y axis) over time (X axis) after addition of NK cells. This data showed that the addition of Dasatinib to NK cells culture for 48 hours or 72 hours duration prior to cryopreservation enhanced the NK cell's anti-tumor cytotoxicity upon thawing compared to NK cells that were not deactivated prior to cryopreservation.

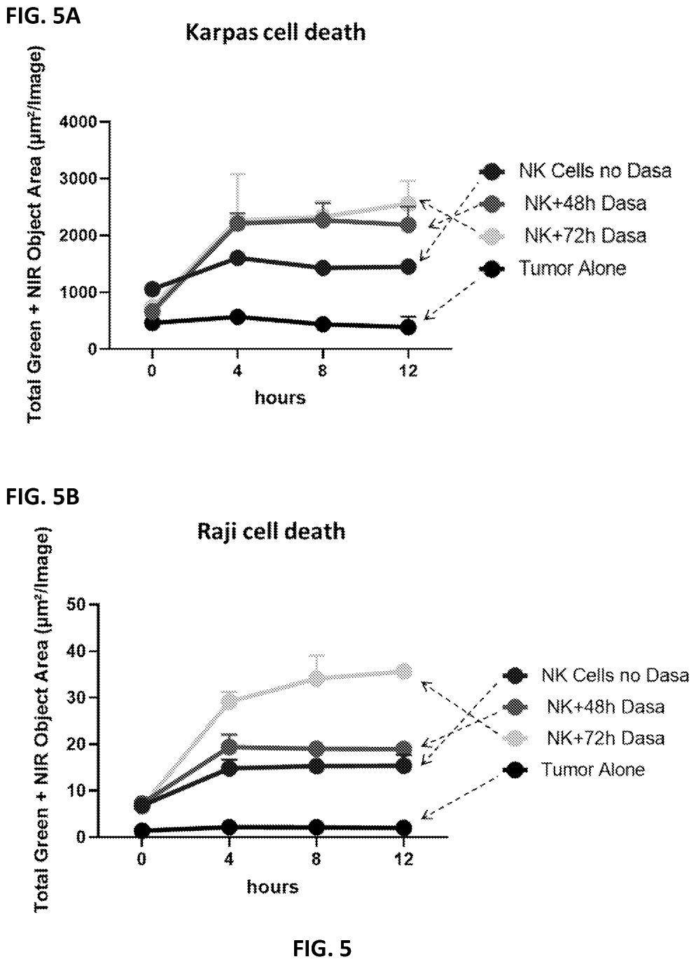

As shown in FIGS. 5A-5B, addition of Dasatinib for 48 or 72 hours pre-cryopreservation improved NK cell anti-tumor cytotoxicity post thaw. FIG. 5A is a graph showing Karpas cell (e.g., Karpas-299 cell line, a human non-hodgkin's Ki-positive large cell lymphoma cell line) death following co-culturing with NK cells that were either deactivated pre-cryopreservation or not deactivated pre-cryopreservation. Cytotox green dye was used to measure Karpas cell death, and green total integrated intensity as a function of Near Infrared Camera (NIR) object area was used as a surrogate for Karpas cell death (Y axis) over time (X axis) after addition of thawed NK cells at an effector target ratio (E:T ratio) of 1:1. FIG. 5B, is a graph showing Raji cell (e.g., a human B lymphoblastoid cell line) death following co-culturing with NK cells that were either deactivated pre-cryopreservation or not deactivated pre-cryopreservation. Cytotox green dye was used to measure Raji cell death, and green total integrated intensity as a function of Near Infrared Camera (NIR) object area was used as a surrogate for Raji cell death (Y axis) overtime (X axis) after addition of NK cells at an effector target ratio (E:T ratio) of 1:1. Together these results demonstrated that addition of Dasatinib to the culture of NK cells prior to cryopreservation enhanced their anti-tumor cytotoxicity upon thawing compared to NK cells that were not deactivated prior to cryopreservation. Thus, the exemplary NK cells that were deactivated pre-cryopreservation can provide an off-the-shelf source of NK cells that can recognize and attack many cancers including both liquid and solid tumors.

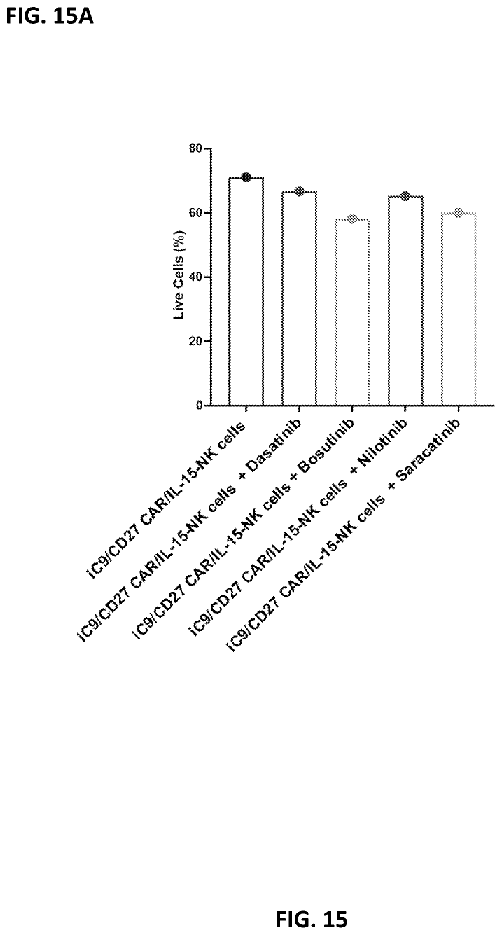

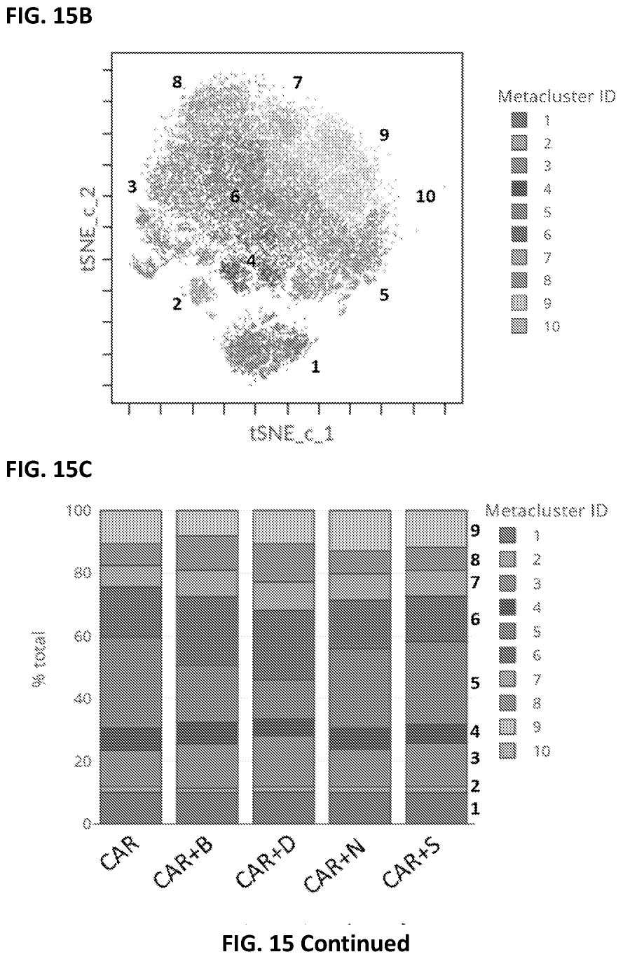

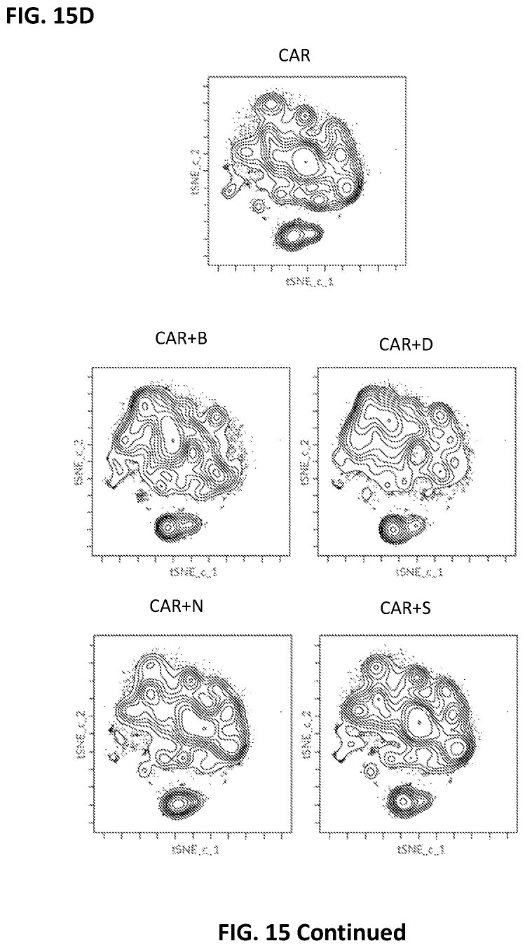

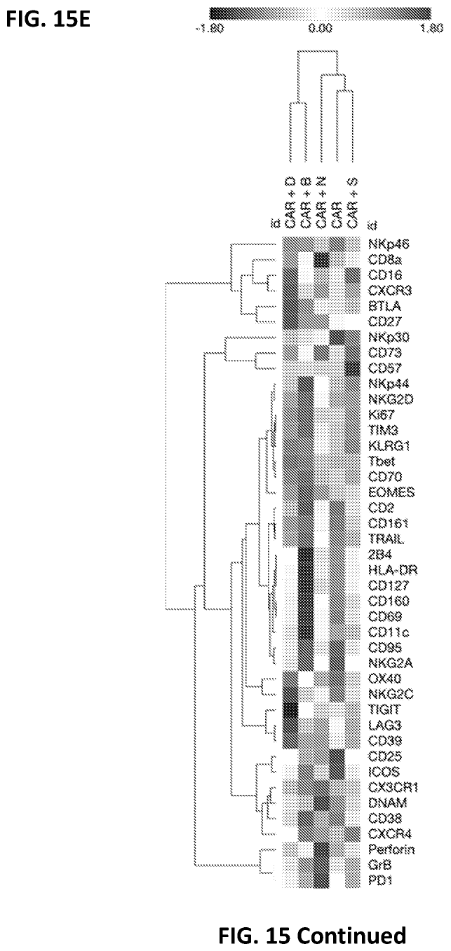

As shown in FIGS. 15A-15E, the effects of pre-cryopreservation treatment with various receptor tyrosine kinase inhibitors (Dasatinib, D; Bosutinib, B; Nilotinib, N; and Saracatinib, S; respectively) on NK cell viability and phenotypes after thawing were analyzed. FIG. 15A depicts the recovery (survival) of NK cells following freeze/thawing where cells were treated with various tyrosine kinase inhibitors (Dasatinib, Bosutinib, Nilotinib, or Saracatinib; at a final concentration of 1 micromolar) pre-cryopreservation. Annexin V assays indicated the percentage of live anti-CD70 CAR NK cells (iCas9/CD27 CAR/IL-15) following freezing and thawing. FIG. 15B depicts t-SNE analysis of thawed iC9/CD27 CAR/IL-15-NK cells (CAR) that were pre-treated with tyrosine kinase inhibitors (Dasatinib (D), Bosutinib (B), Nilotinib (N), or Saracatinib (S)) before cryopreservation; controls included untreated and cryopreserved CAR NK cells. Cells were evaluated post-thawing by CyTOF and merged to create a single t-SNE CUDA map (12,000 from 2 pooled donors per condition). FlowSOM analysis of the various conditions was then performed and the different FlowSOM metaclusters overlapped on the t-SNE CUDA map. Each colored region corresponded to a metacluster (1-10). FIG. 15C depicts a stacked bar graph with relative percentage frequencies of the different FlowSOM metaclusters for each of the CAR NK-cell conditions. FIG. 15D depicts contour plots showing the t-SNE CUDA cluster organization (as depicted in 15B) for the various CAR NK-cell conditions tested. FIG. 15E depicts a representative heatmap showing the expression levels of phenotypic and functional markers for the thawed CAR-NK cells. The Z-score for the expression level for each marker was represented by a color scale with bright red corresponding to the highest expression and dark blue to the lowest expression.

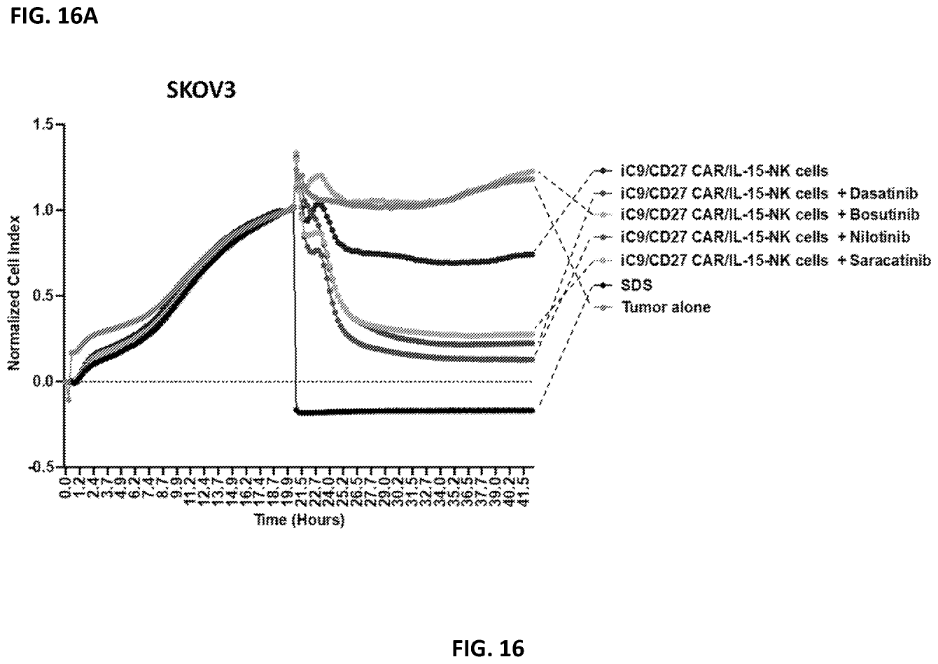

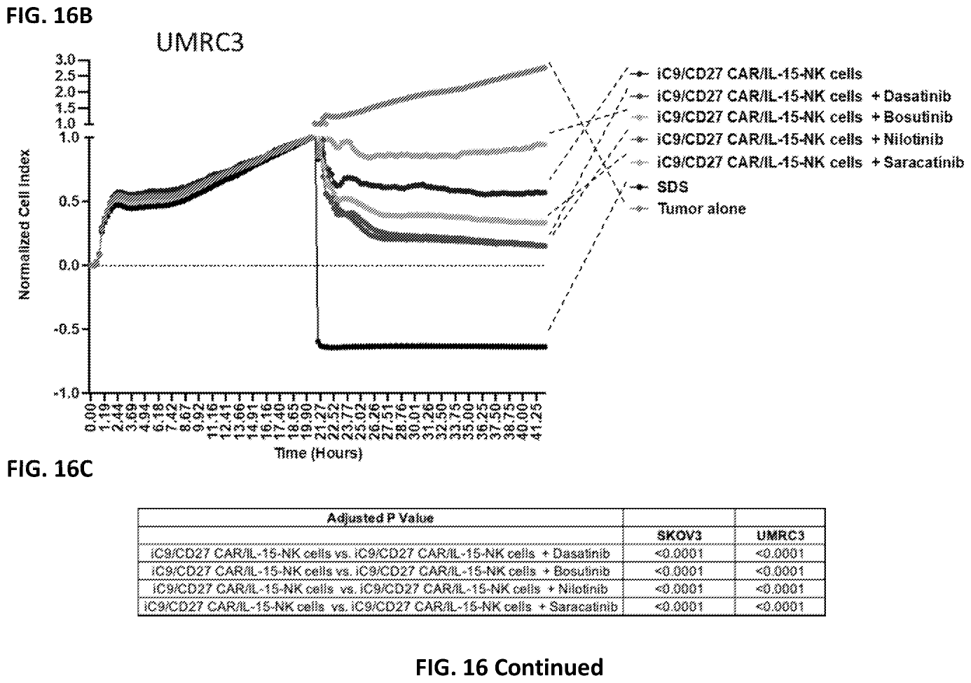

As shown in FIGS. 16A-16C, addition of various tyrosine kinase inhibitors prior to freezing enhanced the antitumor cytotoxicity of CAR-NK cells post-thaw against solid cancer, as determined by xCELLigence impedance assays. The ovarian cancer cell line (SKOV3) (FIG. 16A) and renal cell carcinoma cell line (UMRC3) (FIG. 16B), were grown in 96 well RTCA E-Plates overnight. The next day, frozen iC9/CD27 CAR/IL-15-NK cells that were not treated or were treated with a tyrosine kinase inhibitor (Dasatinib, Bosutinib, Nilotinib, or Saracatinib; at a final concentration of 1 micromolar) pre-cryopreservation were thawed and added at 2:1 effector to target (E:T) ratio. The cancer cell growth was measured continuously over time (X axis) by the xCELLigence device and represented as normalized cell index (Y axis). As shown in FIG. 16C, compared to frozen/thawed iC9/CD27 CAR/IL-15-NK cells without any pre-treatment, frozen/thawed iC9/CD27 CAR/IL-15-NK cells that were pre-treated with tyrosine kinase inhibitors Dasatinib, Nilotinib, or Saracatinib, showed increased cytotoxicity against SKOV3 cells and/or UMRC3 cells.

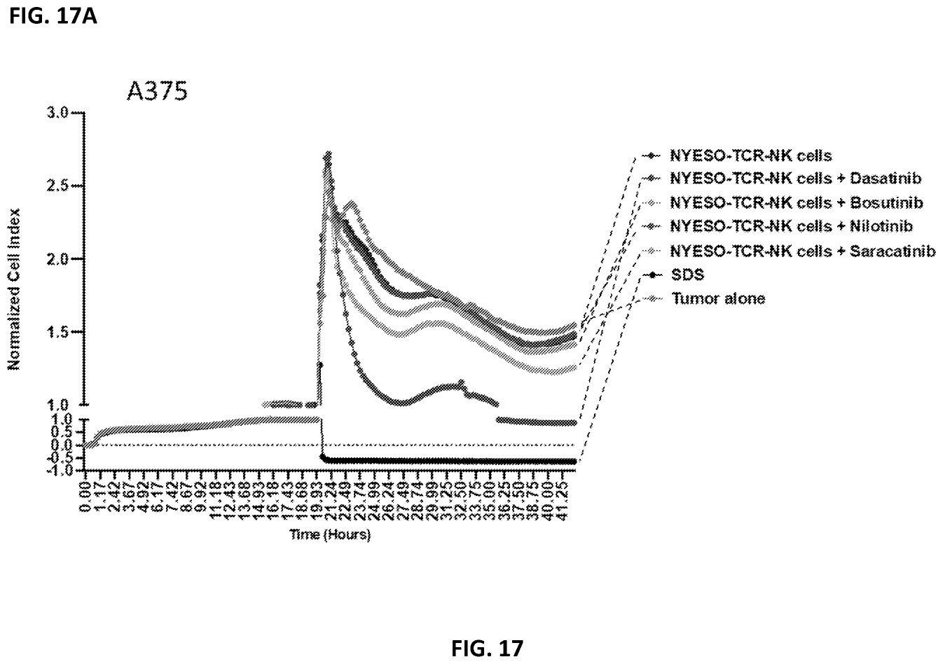

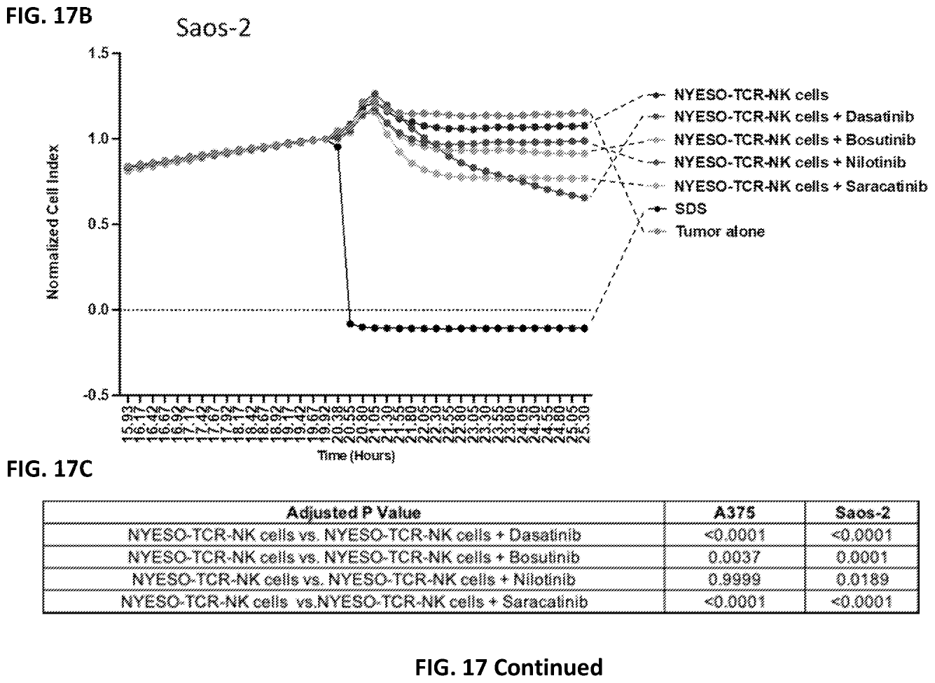

As shown in FIGS. 17A-17C, addition of various tyrosine kinase inhibitors prior to freezing enhanced the antitumor cytotoxicity of TCR-NK cells post-thaw against solid cancer, as determined by xCELLigence impedance assays. The melanoma cell line (A375) (FIG. 17A) and osteosarcoma cell line (Saos-2) (FIG. 17B), were grown in 96 well RTCA E-Plates overnight. The next day, frozen NYESO targeting TCR-NK cells that were not treated or were treated pre-cryopreservation with a tyrosine kinase inhibitor (Dasatinib, Bosutinib, Nilotinib, or Saracatinib; at a final concentration of 1 micromolar) were thawed and added at 2:1 effector to target (E:T) ratio. The cancer cell growth was measured continuously over time (X axis) by the xCELLigence device and represented as normalized cell index (Y axis). As shown in FIG. 17C, compared to frozen/thawed NYESO-TCR-NK cells without any pre-treatment, frozen/thawed NYESO-TCR-NK cells pre-treated with tyrosine kinase inhibitors Dasatinib, Nilotinib, Bosutinib, or Saracatinib showed increased cytotoxicity against A375 cells and/or Saos-2 cells.

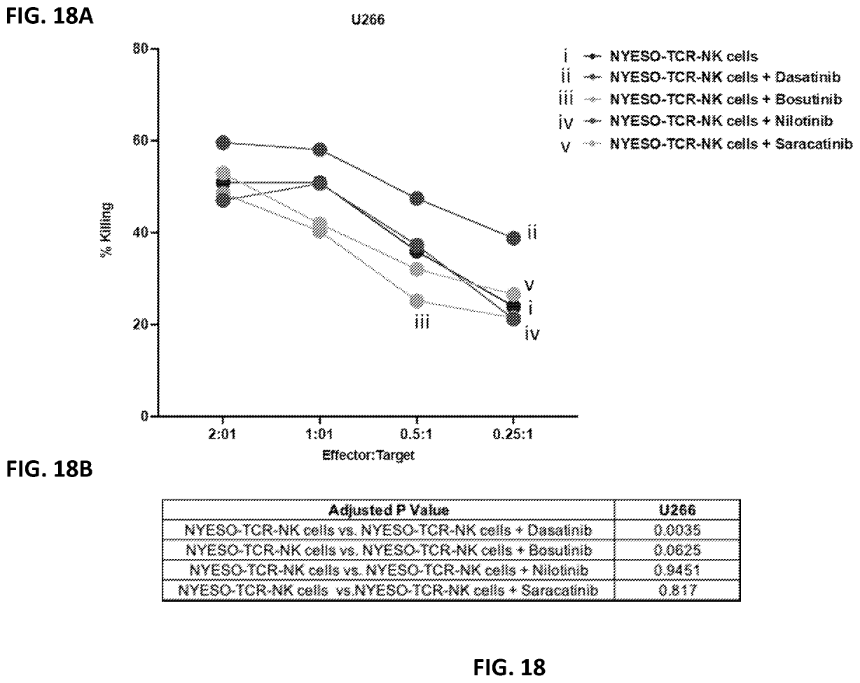

As shown in FIGS. 18A-18B, the addition of Dasatinib prior to freezing increased the post-thaw antitumor cytotoxicity of NYESO targeting TCR-NK cells against multiple myeloma cells as shown by chromium release assays. FIG. 18A depicts cytotoxicity of NYESO TCR NK cells against multiple myeloma cells (U266), the U266 were labelled with chromium-51 and co-cultured with frozen and thawed NYESO-TCR-NK cells that were either treated with various tyrosine kinase inhibitors (Dasatinib, Bosutinib, Nilotinib, or Saracatinib) pre-cryopreservation or not treated pre-cryopreservation, at various E:T ratios (2:1, 1:1, 0.5:1, or 0.25:1). After four hours, chromium release was measured, which corresponded to cancer cell death. As shown in FIG. 17B, Compared to NYESO-TCR-NK cells that were frozen without any pre-treatment, frozen NYESO-TCR-NK cells treated pre-cryopreservation with Dasatinib showed increased cytotoxicity against U266 cells.

Together, these data showed that frozen/thawed NK cells transduced with a transgenic targeting moiety (e.g., a CAR and/or a TCR) displayed enhanced in-vitro cytotoxicity against numerous cancer cell types, including solid cancer cell types, when the NK cells were treated with a deactivating agent pre-cryopreservation.

### Example 4—Deactivation Pre-Cryopreservation Improves NK Cell Cytotoxicity In-Vitro, Improves NK Cell Engraftment Rates, and Improves Probability of Subject Survival

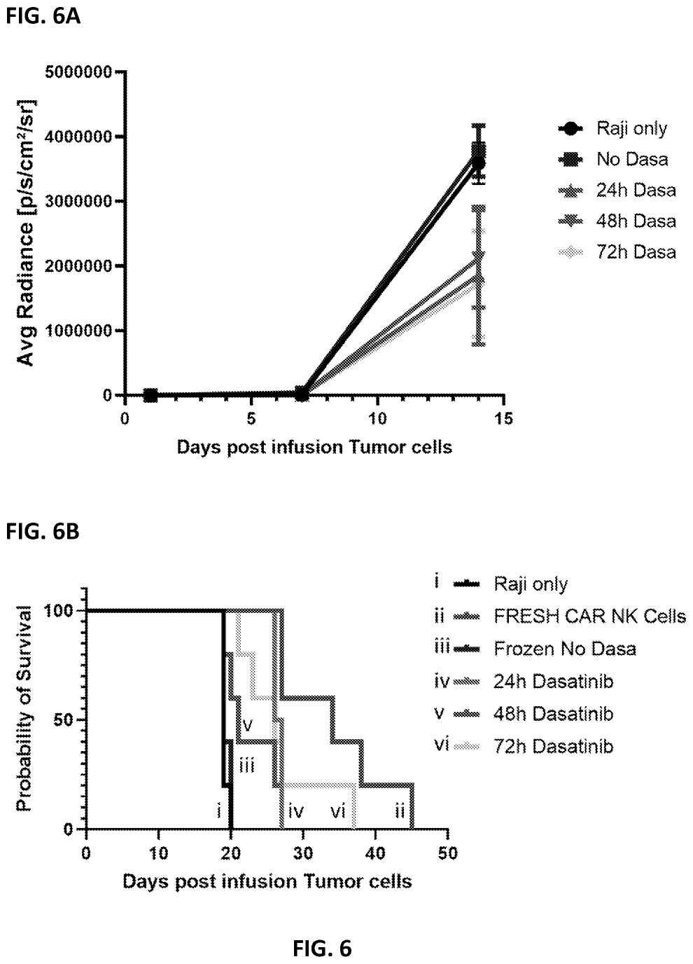

As shown in FIGS. 6A-6B addition of Dasatinib pre-cryopreservation improved NK cell anti-tumor cytotoxicity in vivo and improved survival rates in Raji-NSG (e.g., NOD scid gamma genotype) mice. FIG. 6A is a graph showing the average radiance of tumors (p/s/cm²/sr, a surrogate for tumor growth) (Y axis) over time (days, X axis) in Raji-NSG mice following Raji tumor cell infusion and concurrent treatment with thawed NK cells (approximately 1×10⁷ NK cells). The results showed that NK cells treated with Dasatinib for 24, 48, or 72 hours pre-cryopreservation displayed improved inhibition of tumor growth relative to mice treated with thawed NK cells that were not treated with Dasatinib pre-cryopreservation or mice with tumors that were not treated with NK cells. FIG. 6B is a graph showing the probability of survival (Y axis) of Raji-NSG mice infused with Raji tumor cells and NK cells or control media over time (X axis). The results showed that thawed NK cells treated with Dasatinib for 24, 48, or 72 hours pre-cryopreservation displayed improved inhibition of tumor growth relative to mice treated with thawed NK cells that were not treated with Dasatinib or mice with tumors that were not treated with NK cells.

### Example 5—Deactivation Pre-Cryopreservation Improves NK Cell Cytotoxicity In-Vivo, Improves NK Cell Engraftment Rates, and Improves Probability of Subject Survival

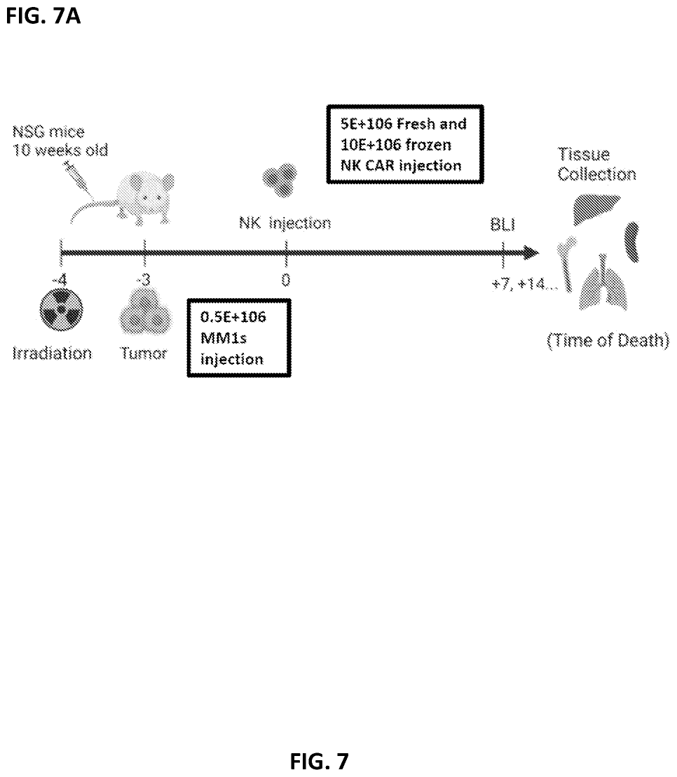

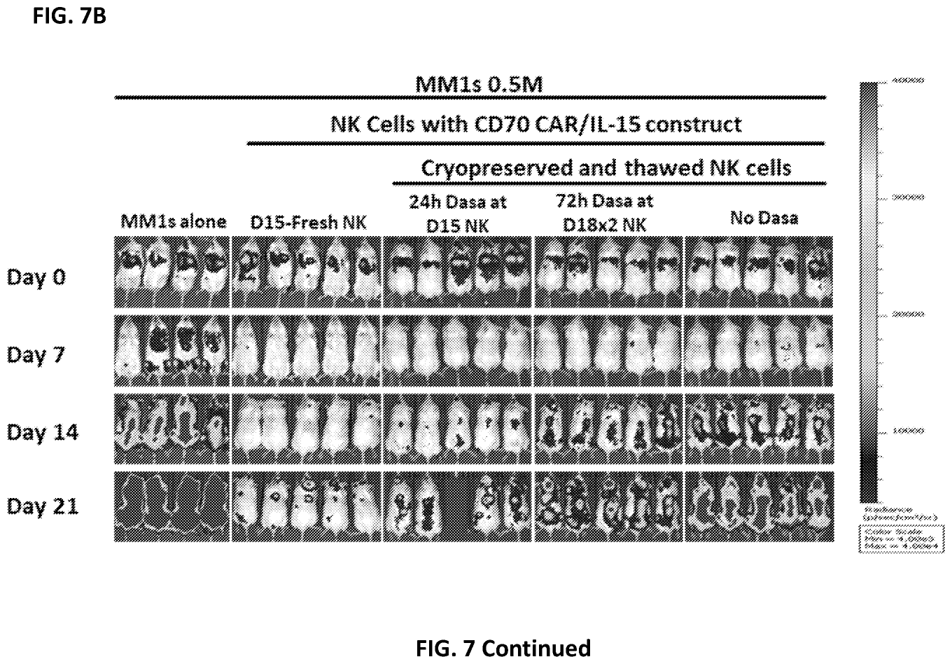

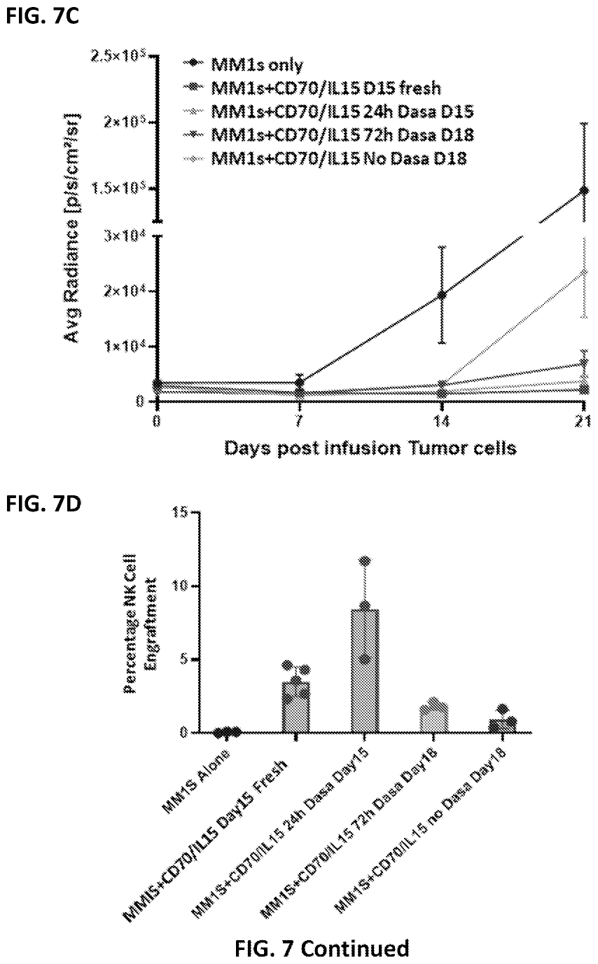

As shown in FIG. 7 addition of Dasatinib at day 14 pre-cryopreservation for 24 hrs or 72 hrs improved NK cell anti-tumor cytotoxicity and NK cell engraftment in vivo. (A) is a schematic outlining the experimental procedure performed. In brief, NSG mice were irradiated on day-1, then on day 0 individual mice received tail vein injections of 0.5×10⁶ MM1S tumor cells (CD70+ multiple myeloma tumor cells) that were transduced with FireFlyluciferase (Ffluc), on day 3 mice were infused with 5×10⁶ fresh CAR NK cells or 10×10⁶ frozen & subsequently thawed CAR NK cells (NK cells comprising constructs comprising a CD70 specific CAR and IL-15), the frozen & subsequently thawed CAR NK cells were treated on day 14 of culture for 24 hrs or 72 hrs with Dasatinib pre-cryopreservation, or not treated with a deactivating agent pre-cryopreservation. Animals were then monitored over time and sacrificed as appropriate (N=4 or 5 mice per group). (B) Displays the results of the monitoring of the experiment described in (7A) as a function of bioluminescent imaging over time (displayed are representative images from day 0, day 7, day 14, and day 21 respectively). (C) is a graphical quantification of the bioluminescence average radiance displayed in (7B), the Y axis denotes average radiance in p/s/cm²/sr, while the X axis denotes time. (D) is a graphical quantification of the percentage of in vivo NK cell engraftment on day 10 following NK cell infusion into the mice displayed in (7B).

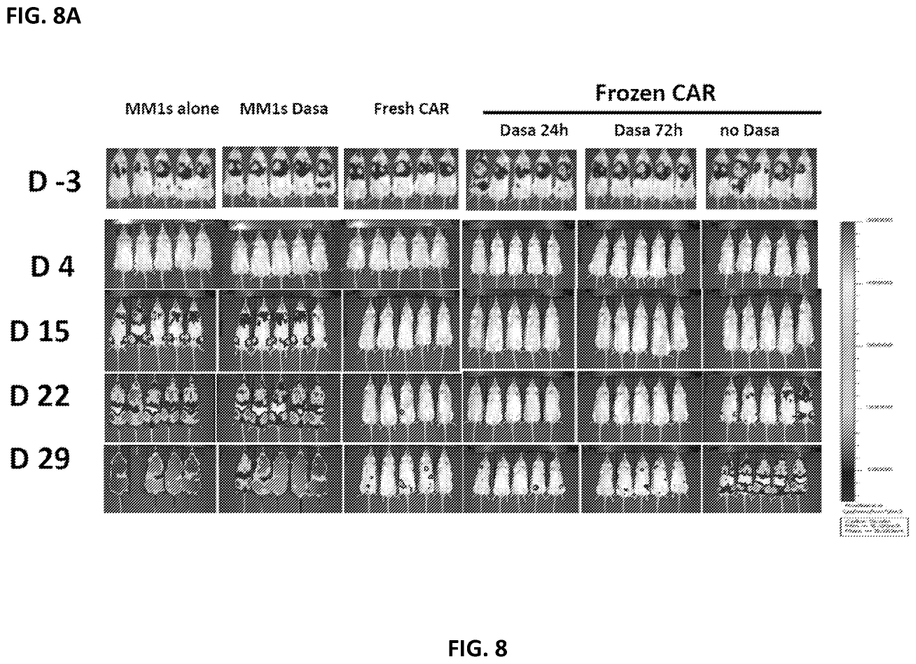

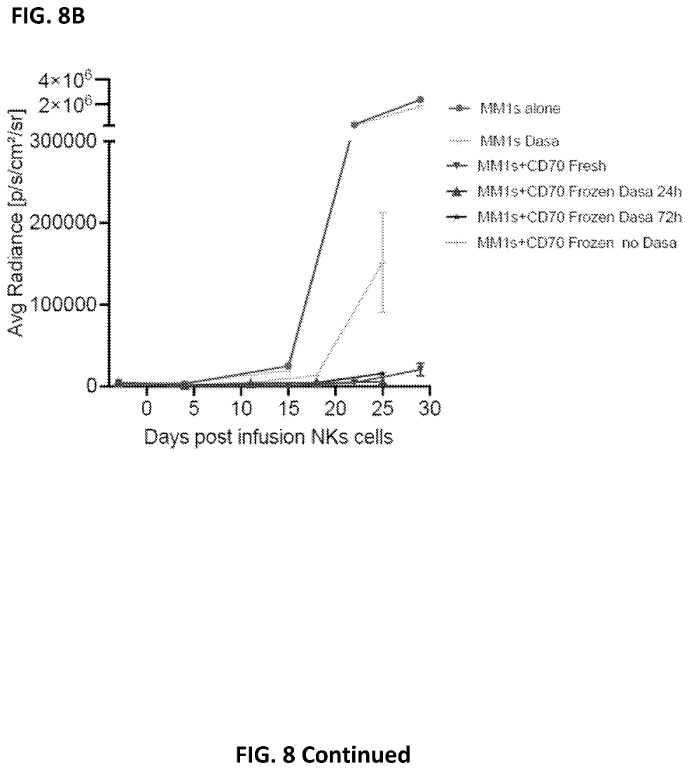

As shown in FIGS. 8A-8B, CAR NK cells cultured for 14 days and then treated with Dasatinib for 24 or 72 hours prior to freezing were superior to CAR NK cells stored with standard freezing media protocols without deactivation, and the pre-cryopreservation treated CAR NK cells displayed in vivo anti-tumor activity that was comparable to that of fresh CAR NK cells (at day 14 of culture). (A) Bioluminescence imaging (BLI) showing MM1S (CD70+ multiple myeloma cells, transduced with FireFlyluciferase (FFluc)) tumor growth over time (day −3, day 4, day 15, day 22, and day 29) in mice that received MM1S cells alone, MM1S cells plus Dasatinib, MM1S cells plus fresh CD70-targeting CAR-NK cells, or MM1S cells plus frozen CAR-NK cells either treated pre-cryopreservation with Dasatinib for 24 hours or 72 hours or not treated pre-cryopreservation. (B) is a graphical quantification of the bioluminescence average radiance displayed in (8A). The findings demonstrated that anti-CD70 CAR NK cells deactivated with dasatinib before being frozen performed as well as fresh CAR NK cells and better than CAR NK cells that had previously been frozen but had not been deactivated before being frozen.

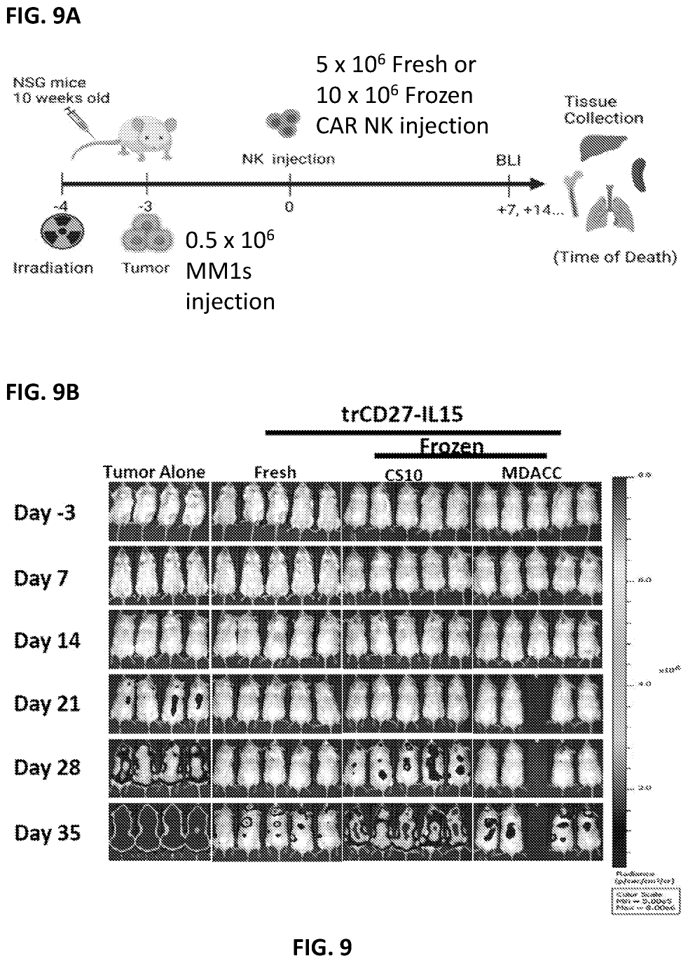

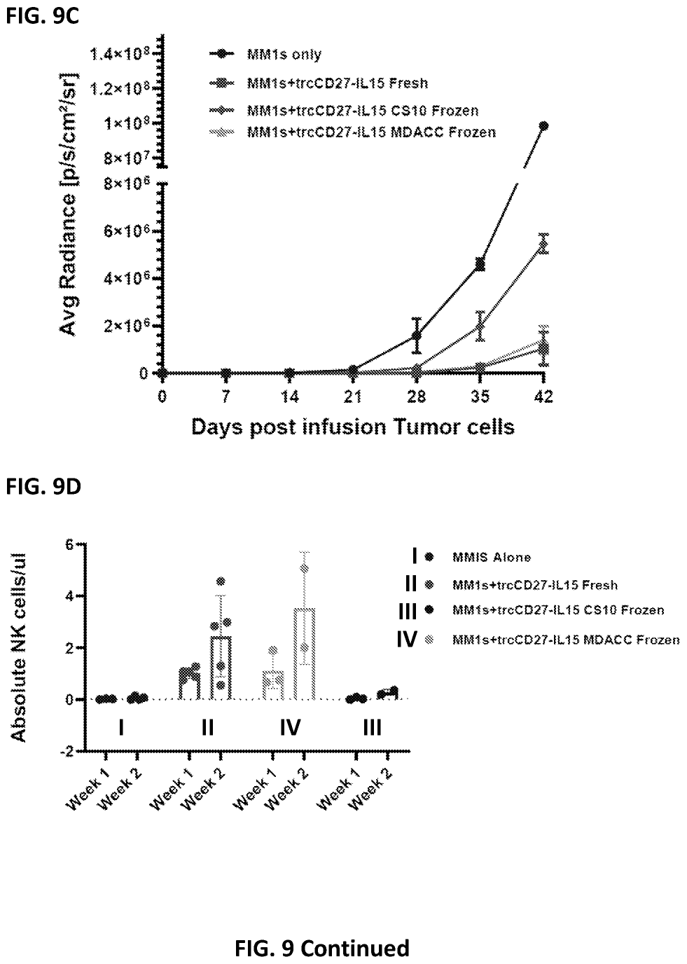

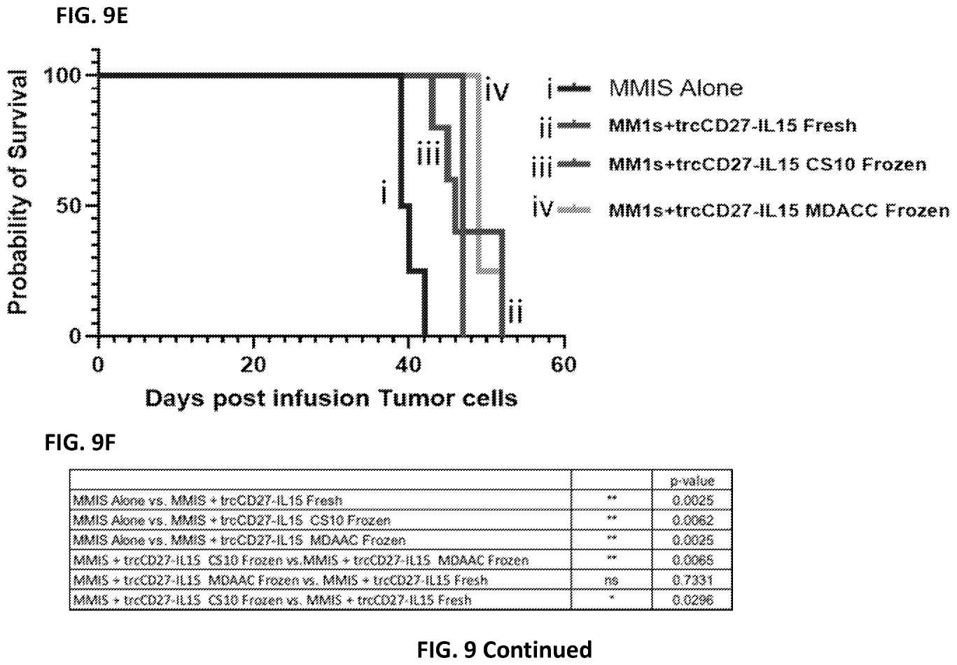

As shown in FIGS. 9A-9E, CAR NK cells cultured for 13 days and then treated with Dasatinib for 24 hours prior to freezing were superior to CAR NK cells stored with standard freezing media protocols without deactivation. Furthermore, the Dasatinib-treated cells showed in vivo anti-tumor control comparable to that of fresh (day 13 of culture) CAR NK cells. (A) is a diagram illustrating the experimental procedure (animals were irradiated on day −4, injected with 0.5×10⁶ MM1S cells on day −3, injected with 5×10⁶ fresh CAR NK cells or 10×10⁶ frozen CAR NK cells on day 0, and imaged weekly). (B) Bioluminescence imaging (BLI) showing MM1S(CD70+ multiple myeloma cells, transduced with FireFlyluciferase (FFluc)) tumor growth over time (day −3, day 4, day 15, day 22, and day 29) in mice that received MM1S cells alone or tumor plus Dasatinib or tumor plus fresh CD70-targeting CAR-NK cells or tumor plus frozen CAR-NK cells either pre-treated with dasatinib for 24 hours or not. (C) is a graphical quantification of the bioluminescence average radiance displayed in (9B). (D) is a graphical quantification of the absolute count of in vivo NK cell engraftment in the blood of mice 10 days or 20 days after receiving CAR-NK cells displayed in (9B). (E) displays the survival curves of the four groups of mice displayed in (9B), while (F) displays the associated statistical analysis results. These findings demonstrated that NK cells deactivated prior to cryopreservation could effectively control tumor growth, engraft, and increase survival at comparable levels to those of fresh NK cells, and that NK cells that were deactivated before cryopreservation inhibited tumor growth and engrafted more effectively than NK cells that were not deactivated before cryopreservation.

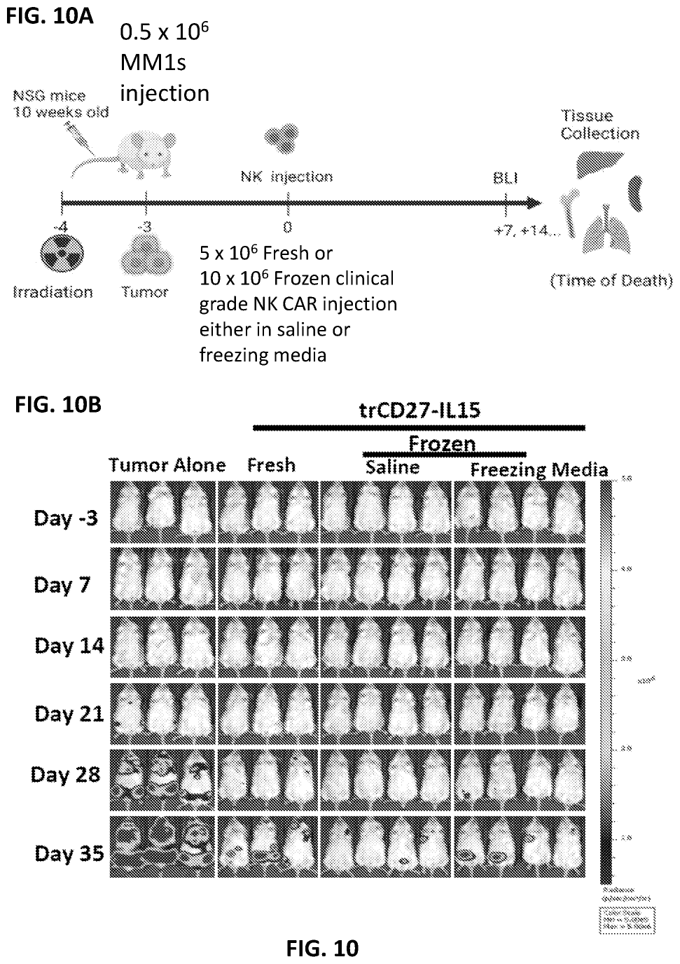

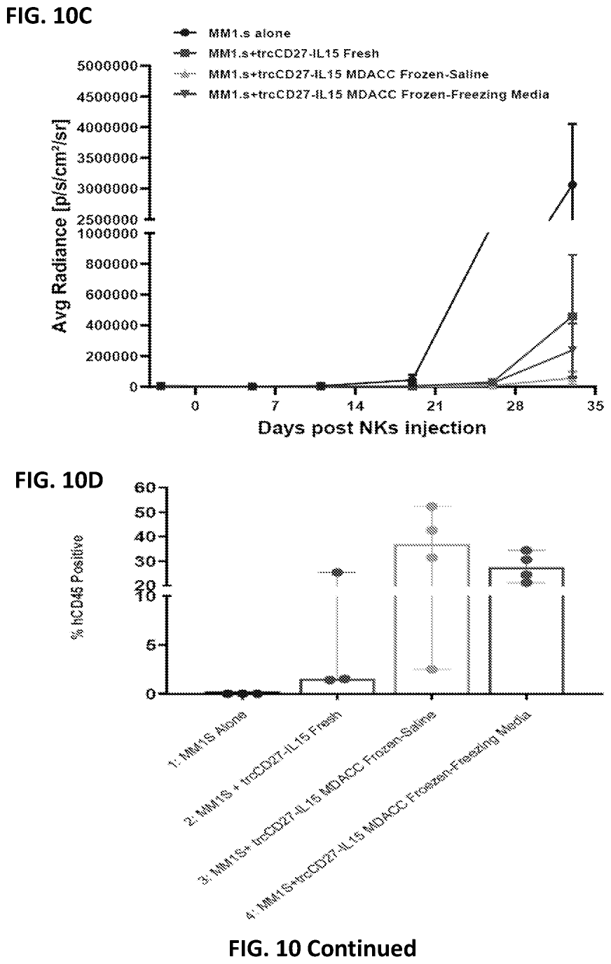

As shown in FIG. 10, CAR NK cells that were cultured for 13 days and then treated with Dasatinib for 24 hours before undergoing freezing exhibited comparable in vivo antitumor control and engraftment capacities relative to freshly prepared CAR NK cells (day 13 of culture). Furthermore, the data demonstrated that the activity of CAR NK cells remained unaffected when injected with freezing media. (A) is a diagram illustrating the experimental procedure (animals were irradiated on day-4, injected with 0.5×10⁶ MM1S cells on day −3, injected with 5×10⁶ fresh CAR NK cells or 10×10⁶ frozen CAR NK cells on day 0, and imaged weekly). (B) Bioluminescence imaging (BLI) showing MM1S (CD70+ multiple myeloma cells, transduced with FireFlyluciferase (FFluc)) tumor growth over time (day −3, day 7, day 14, day 21, day 28 and day 35) in mice that received MM1S cells alone, MM1S cells plus fresh CD70-targeting CAR-NK cells, or MM1S cells plus frozen CAR-NK cells that were either injected with saline or freezing media. (C) is a graphical quantification of the bioluminescence average radiance displayed in (10B). (D) is a graphical quantification of the percentage of in vivo NK cell engraftment in the blood of mice 10 days after receiving CAR-NK cells displayed in (10B). These findings demonstrated that NK cells deactivated prior to cryopreservation were able to effectively control tumor growth and engraft in a subject at levels comparable to fresh NK cells, and that NK cells deactivated prior to cryopreservation controlled tumor growth and engrafted better than NK cells that were not deactivated prior to cryopreservation.

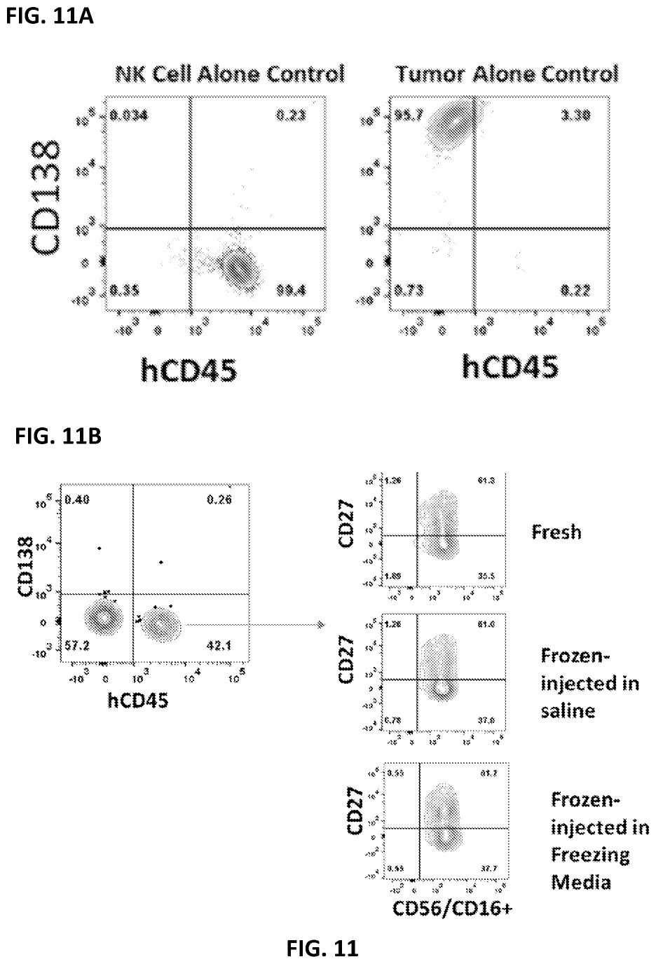

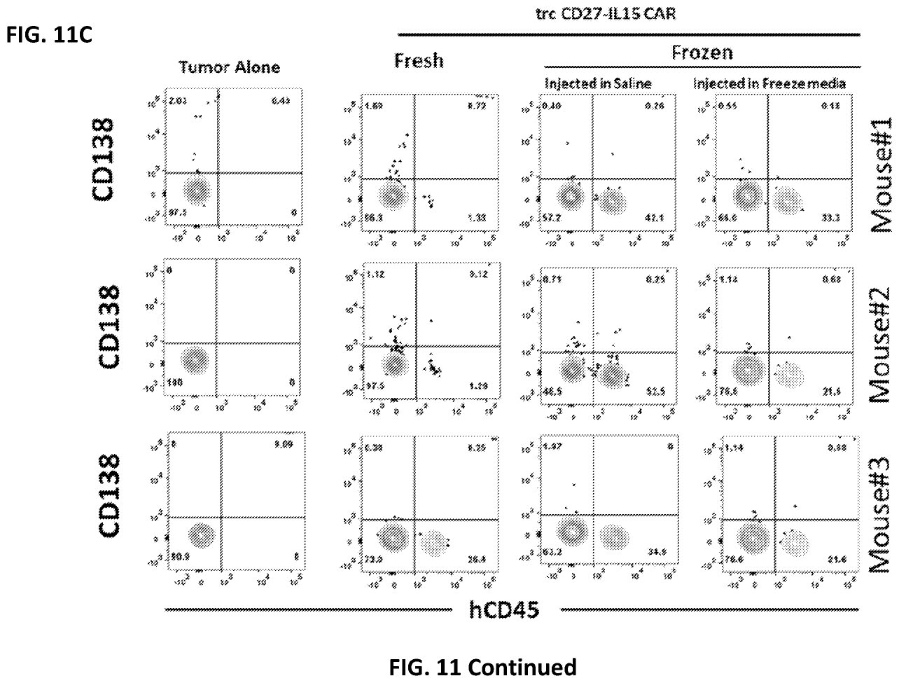

As shown in FIGS. 11A-11C, thawed CAR NK cells that were cryopreserved after being cultured for 13 days and treated with Dasatinib for 24 hours prior to freezing exhibited in vivo engraftment levels that were comparable to those of freshly prepared CAR NK cells at day 10 following infusion. Furthermore, the data demonstrated that the engraftment rates of CAR NK cells remained unaffected when injected with freezing media. (A) Using negative and positive controls for CD138 (a tumor marker), and hCD45 (a natural killer cell marker), the quality of the antibodies and the gating strategy were evaluated. (B) Demonstrates the percentages of CD56/CD16+ and CD27+(CAR marker) cells that were previously gated as CD138− and hCD45+(left; example panel), fresh CD70-targeting CAR-NK cells (top right), or frozen CAR-NK cells either injected with saline (middle right) or freezing media (bottom right). (C) Demonstrates the percentages of hCD45+/CD138− (CAR NK cells) engraftment from 3 different mice that had been injected with tumor plus fresh CD70-targeting CAR-NK cells, or tumor plus pre-cryopreservation Dasatinib treated CAR-NK cells that were thawed and either injected with saline or freezing media.

Together these results showed that treatment with a deactivating agent pre-cryopreservation improved NK cell cytotoxicity against tumor cells, and improved engraftment percentage in-vivo.

### Example 6—Deactivation Pre-Cryopreservation Preserves NK Cell Cytotoxicity In-Vivo or Ex-Vivo for Various Cancer Types

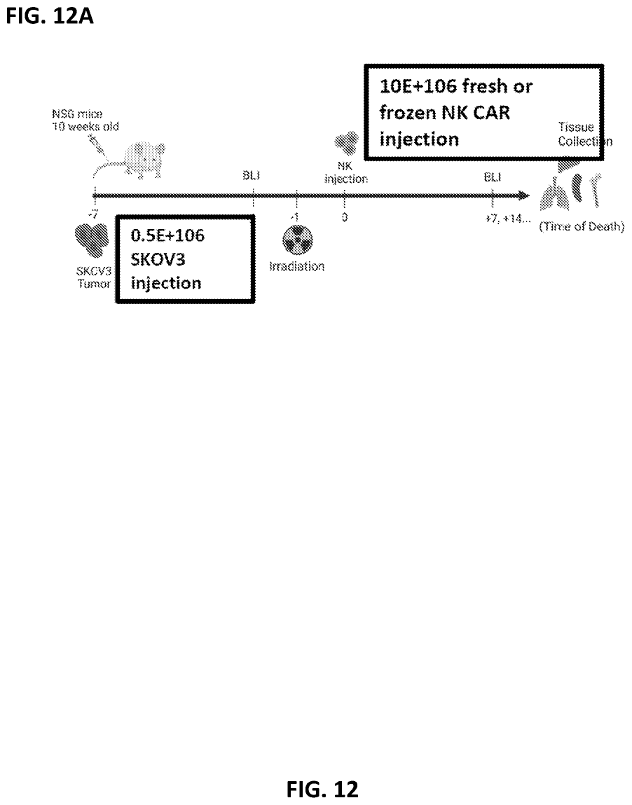

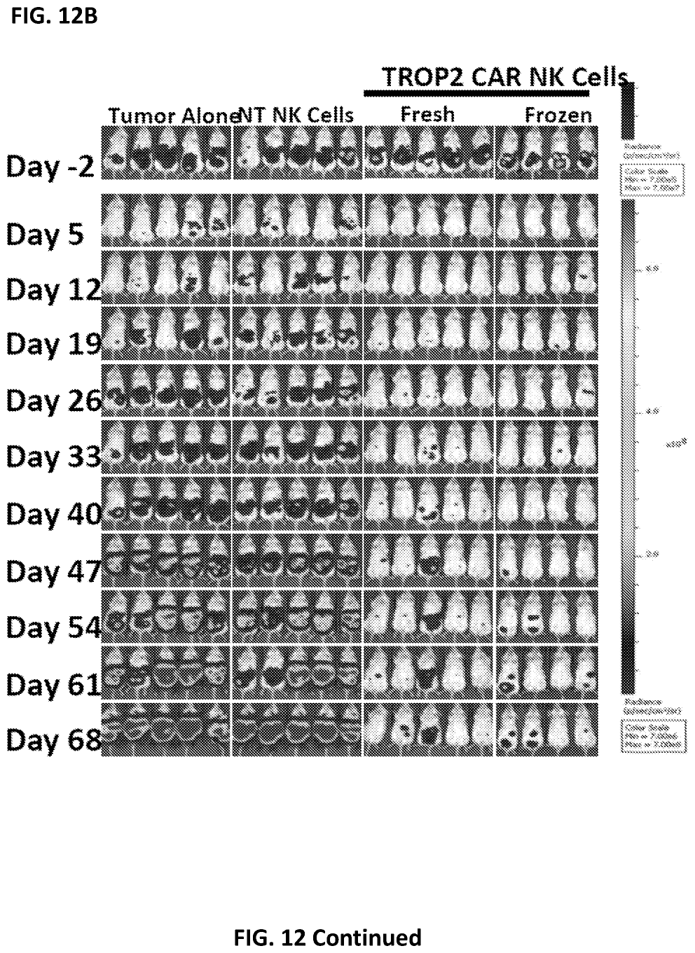

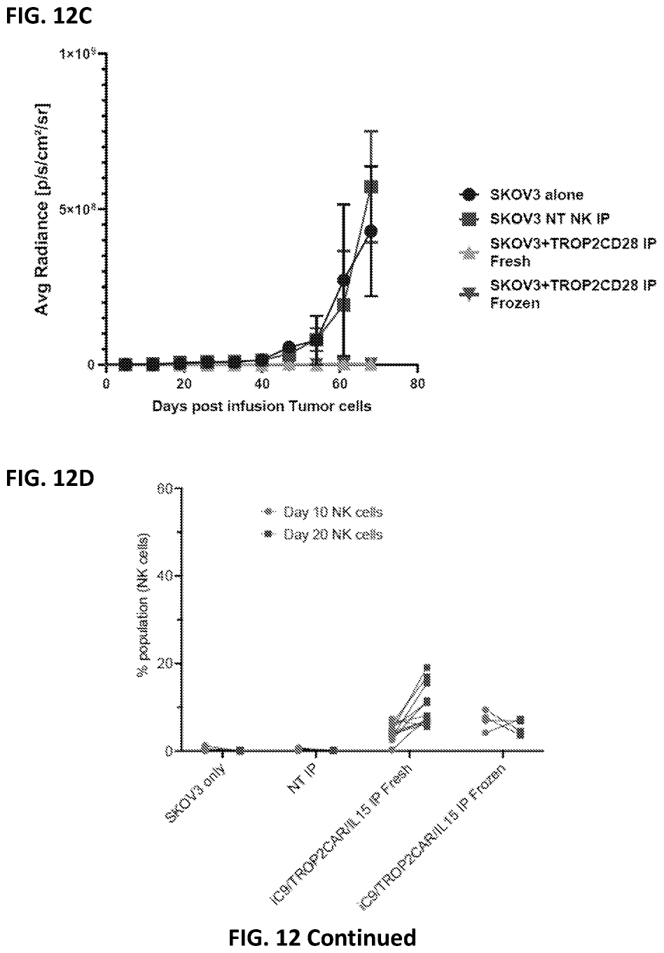

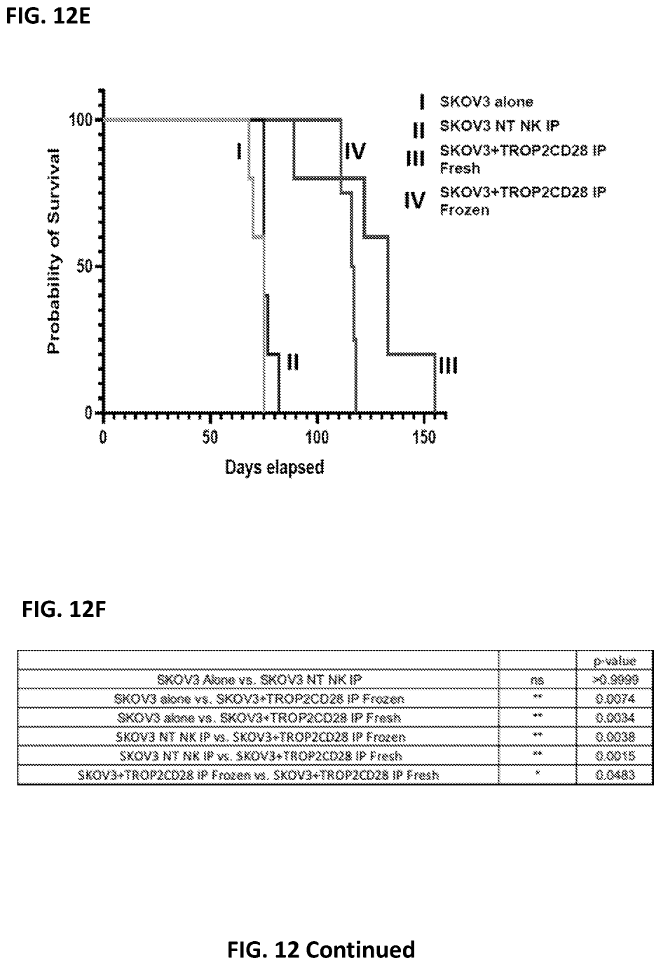

As shown in FIG. 12, NK cells treated with the addition of Dasatinib prior to freezing demonstrated antitumor cytotoxicity that was comparable to fresh NK cells in-vivo in an ovarian cancer model. (A) is a schematic outlining the experimental procedure performed (animals were injected with 0.5×10⁶ SKOV3 cells on day −7; BLI began on day-2, animals were irradiated on day-1, and injected with 10×10⁶ fresh CAR NK cells or 10×10⁶ frozen CAR NK cells on day 0; and imaged weekly throughout life). (B) Bioluminescence imaging (BLI) showing SKOV3 (TROP2+ ovarian cancer cells, transduced with FireFlyluciferase (FFluc)) tumor growth over time (day-2, day 5, day 12, day 19, day 26, day 33, day 40, day 47, day 54, day 61, and day 68) in mice that received SKOV3 cells alone, SKOV3 cells plus NT NK cells, SKOV3 cells plus TROP2-targeting CAR-NK cells, or SKOV3 cells plus pre-cryopreservation Dasatinib treated frozen/thawed TROP2-targeting CAR-NK cells. (C) is a graphical quantification of the bioluminescence average radiance displayed in (12B). (D) is a graphical quantification of the percentage of in vivo NK cell engraftment in the blood of mice 10 days or 20 days after receiving the NK cells displayed in (12B). (E) displays the survival curves of the four groups of mice in (12B), and (F) is the associated statistical analysis. Together, these data demonstrated that NK cells deactivated prior to cryopreservation could effectively control tumor growth (e.g., ovarian cancer cells), engraft, and improve subject survival at levels comparable to fresh NK cells.

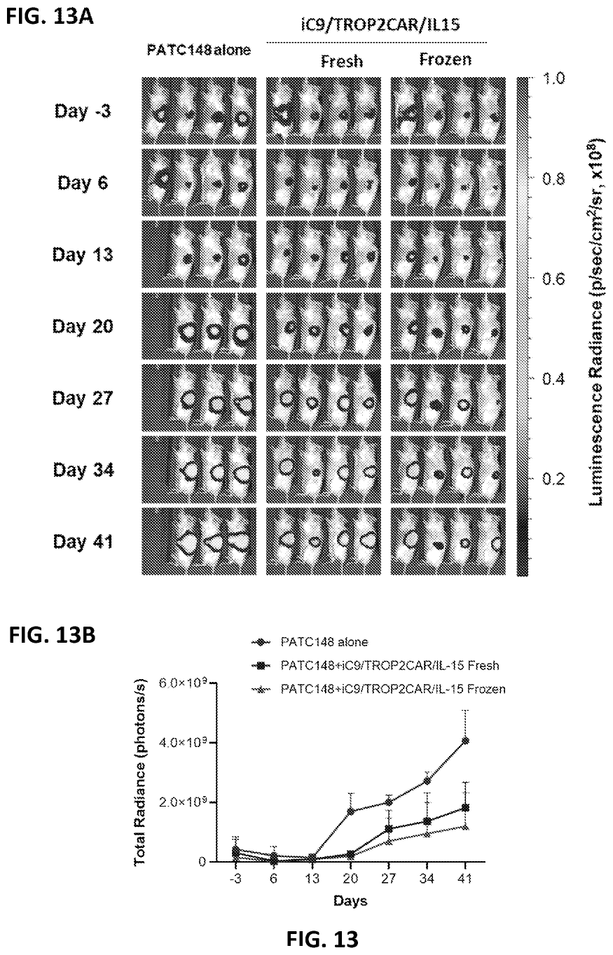

As shown in FIG. 13, NK cells treated with the addition of Dasatinib prior to freezing demonstrated antitumor cytotoxicity that was comparable to fresh NK cells in-vivo in a pancreatic ductal adenocarcinoma (PATC148) mouse model. (A) displays bioluminescence imaging over time (day 3, day 6, day 13, day 20, day 27, day 34, and day 41) for the mice engrafted with PATC148 cells transduced with FireFlyluciferase (FFluc) with no treatment (PATC148 alone), fresh NK cells transduced with a construct comprising TROP2-targeting CAR (iC9/TROP2CAR/1L15), or frozen CAR-NK cells comprising the same construct that were treated with Dasatinib prior to freezing. (B) is a graphical quantification of the bioluminescence average radiance displayed in (13A). Together, these data demonstrated that NK cells deactivated prior to cryopreservation could effectively control tumor growth (e.g., pancreatic cancer cells) at levels comparable to fresh NK cells.

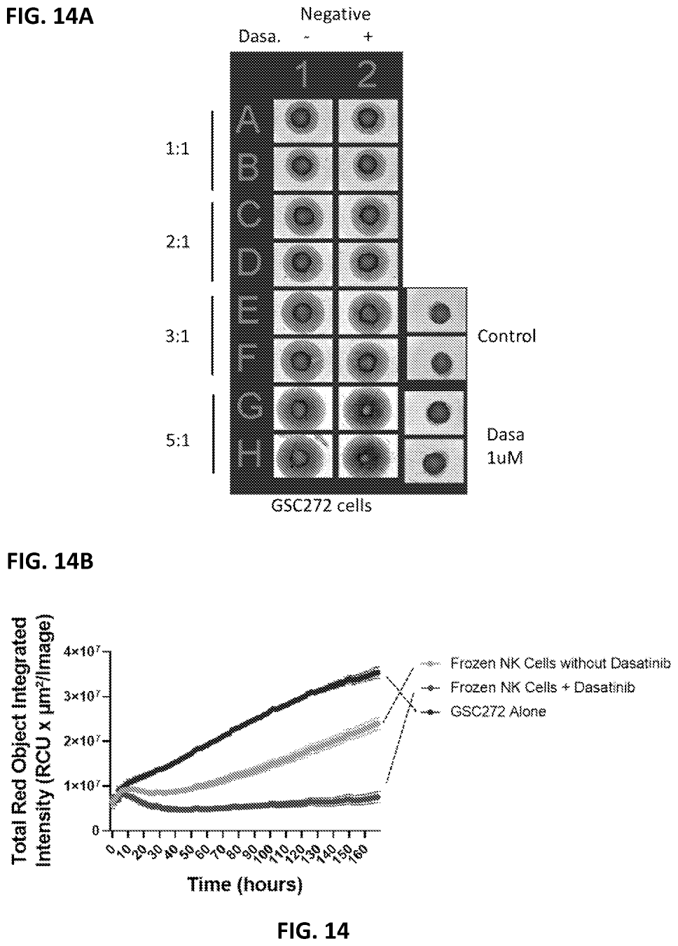

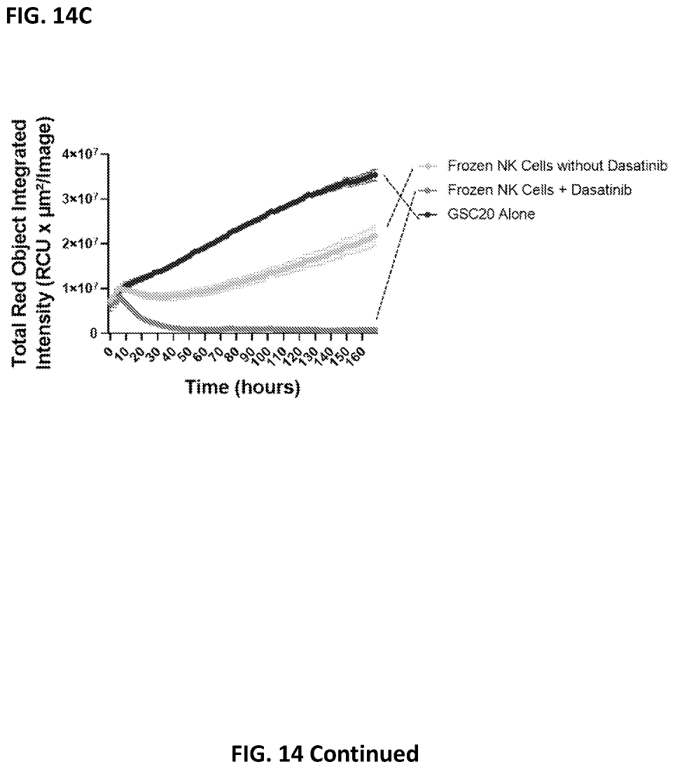

As shown in FIG. 14, the addition of Dasatinib to NK cells prior to freezing enhanced their antitumor cytotoxicity post-thawing in in vitro glioblastoma tumor spheroid assays. (A) displays representative images of GSC272 spheroids (glioblastoma multiforme (GBM) tumor cell cancer stem cell lines transduced with mcherry) cultured alone (right column, top 2 panels), co-cultured with NK cells without Dasatinib treatment prior to freezing (column 1), or co-cultured with NK cells treated with Dasatinib prior to freezing (column 2), at E:T ratios of 1:1, 2:1, 3:1, or 5:1. Dasatinib alone was also added to some tumor cell comprising wells as control (right column, bottom 2 panels). (B) depicts quantification of total integrated red intensity over time showing a significant decrease in the total integrated red intensity (as a measure of tumor growth) when spheroids were co-cultured with NK cells that had been deactivated with Dasatinib prior to freezing. (C) depicts quantification of total integrated red intensity over time showing a significant decrease in the total integrated red intensity (as a measure of tumor growth) when GSC20 spheroids (GBM tumor cell cancer stem cell lines transduced with mcherry) cultured alone, co-cultured with NK cells without Dasatinib treatment prior to freezing, or co-cultured with NK cells treated with Dasatinib prior to freezing. Together, these data demonstrated that NK cells deactivated prior to cryopreservation could more effectively control tumor growth (e.g., GBM cancer cells) when compared to NK cells that were not deactivated prior to cryopreservation.

### Example 7—Exemplary Method of Cryopreserving NK Cells Through Pre-Cryopreservation Deactivation

NK cells (e.g., non-engineered and/or engineered NK cells) are cryopreserved in accordance with aspects of the present disclosure.

An exemplary method of cryopreserving CAR-NK cells through pre-cryopreservation deactivation comprises expanding NK cells from banked umbilical cord blood with uAPC and IL-2; optionally transducing the NK cells with a construct comprising a cytokine and optionally an additional heterologous protein (e.g., a protein comprising a targeting moiety, e.g., a CAR, a TCR, an antibody, etc.) via a retroviral vehicle; culturing and expanding the NK cells; washing the transduced NK cells (e.g., with warm complete media, e.g., RPMI+Clicks+Human AB serum) and subsequently deactivating the NK cells using a deactivating agent; washing the transduced CAR-NK cells (e.g., with HSA PlasmaLyte-A buffer); aliquoting the transduced CAR-NK cells at desired concentrations (e.g., 3×10⁶ cells/mL or 25×10⁶ cells/mL) to individual vials; freezing the transduced CAR-NK cells (e.g., using a control rate freezer); and storing the transduced CAR-NK cells (e.g., in vapor phase in a liquid nitrogen storage unit). Transduced (e.g., engineered) or Non-transduced NK cells may be cryopreserved using this exemplary method. NK cells cryopreserved under this exemplary method can be stored for an extended period of time as frozen NK cell products. The frozen NK cell products can then be conveniently transported to a healthcare provider, properly thawed, and delivered to a patient for treating diseases (e.g., cancer, an autoimmune disease, an infection, etc.).

Utilizing methods and compositions of the disclosure, NK cells were effectively stored to provide an off-the-shelf source of NK cells that can recognize and attack cancers including both liquid and solid tumors. These NK Cells may comprise transgenes including CARs, and displayed improved cell viability, cell cytotoxicity, transgene expression, and subject survival rates relative to NK cells stored using traditional means. The ability to cryopreserve NK cell such that post thaw they retain the same potency as their fresh counterpart is extremely valuable as it will allow for this type of immunotherapy to be used as an off-the-shelf therapy for patients with cancer. It is important to note that NK cells have been traditionally very difficult to freeze and there are currently no efficacious commercial freezing protocols available for the cryopreservation of NK cells.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A method of deactivating a Natural Killer (NK) cell, comprising:
treating an NK cell with an effective amount of one or more deactivating agents under conditions to produce a deactivated NK cell.

2. The method of claim 1, wherein the deactivating agent is a kinase inhibitor.

3. The method of claim 1 or 2, wherein the deactivating agent is a mechanistic target of rapamycin (mTOR) inhibitor.

4. The method of claims 2 or 3, wherein the mTOR inhibitor is rapamycin, everolimus, and/or temsirolimus.

5. The method of any one of claims 2-4, wherein the mTOR inhibitor is rapamycin.

6. The method of claim 1 or 2, wherein the deactivating agent is a tyrosine kinase (TK) inhibitor.

7. The method of claim 6, wherein the TK inhibitor is Dasatinib, Nilotinib, Lorlatinib, Brigatinib, Ceritinib, Alectinib, Crizotinib, Bosutinib, Ponatinib, Saracatinib, Imatinib, Zanubrutinib, Acalabrutinib, Ibrutinib, Capmatinib, Pexidartinib, Dacomitinib, Osimertinib, Erlotinib, Gefitinib, Lapatinib, Afatinib, Pemigatinib, Erdafitinib, Nintedanib, Gilteritinib, Midostaurin, Tucatinib, Neratinib, Baricitinib, Ruxolitinib, Fedratinib, Tofacitinib, Ripretinib, Selumetinib, Binimetinib, Cobimetinib, Trametinib, Upadacitinib, Avapritinib, Selpercatinib, Cabozantinib, Fostamatinib, Larotrectinib, Entrectinib, Axitinib, Regorafenib, Pazopanib, Sorafenib, Lenvatinib, Vandetanib, and/or Sunitinib.

8. The method of claim 6 or 7, wherein the TK inhibitor is a BCR-Abl inhibitor.

9. The method of any one of claims 6-8, wherein the TK inhibitor is Dasatinib, Nilotinib, Bosutinib, Ponatinib, and/or Imatinib.

10. The method of any one of claims 6-9, wherein the TK inhibitor is Dasatinib and/or Nilotinib.

11. The method of any one of claims 6-10, wherein the TK inhibitor is Nilotinib.

12. The method of any one of claims 6-10, wherein the TK inhibitor is Dasatinib.

13. The method of any one of claims 1-12, wherein the treatment is at any point during culturing of the NK cell.

14. The method of any one of claims 1-13, wherein the treatment is for about 24 to about 96 hours, about 36 to about 84 hours, or about 48 to about 72 hours.

15. The method of any one of claims 1-14, wherein the treatment is for about 24 hours, about 48 hours, or about 72 hours.

16. The method of any one of claims 1-15, wherein the treatment is for about 24 hours.

17. The method of any one of claims 1-16, wherein the NK cell is treated with the deactivating agent at a concentration of about 1 to about 2000 nM.

18. The method of any one of claims 1-17, wherein the NK cell is treated with the deactivating agent at a concentration of about 5 to about 100 nM.

19. The method of any one of claims 1-18, wherein the NK cell is treated with the deactivating agent at a concentration of about 20 to about 500 nM.

20. The method of any one of claims 1-19, wherein the NK cell is treated with the deactivating agent at a concentration of about 30 to about 200 nM.

21. The method of any one of claims 1-16, wherein the NK cell is treated with the deactivating agent at a final concentration of about 0.01 μM to about 10 μM.

22. The method of any one of claims 1-16 or 21, wherein the NK cell is treated with the deactivating agent at a final concentration of about 0.02 μM to about 5 μM.

23. The method of any one of claims 1-16 or 21-22, wherein the NK cell is treated with the deactivating agent at a final concentration of about 0.05 μM to about 3 μM.

24. The method of any one of claims 1-16 or 21-23, wherein the NK cell is treated with the deactivating agent at a final concentration of about 1 μM.

25. The method of any one of claims 1-24, wherein the cells are washed following deactivation.

26. The method of any one of claims 1-25, wherein the deactivated NK cell has an increased expression of one or more of C-kit, CCR-5, CD62L and/or CXCR4, and/or decreased expression of one or more of NKG2D, DNAM, OX-40, TRAIL, HLA-DR, CD2, CD25, ICOS, and/or CD95 relative to an activated NK cell.

27. The method of any one of claims 1-26, wherein the NK cell is derived from human peripheral blood mononuclear cells (PBMC), unstimulated leukapheresis products (PBSC), human embryonic stem cells (hESCs), hematopoietic stem cells, induced pluripotent stem cells (iPSCs), bone marrow, an NK cell line, and/or umbilical cord blood.

28. The method of any one of claims 1-27, wherein the NK cell is isolated from blood.

29. The method of claim 28, wherein the NK cell is isolated from one or more umbilical cord blood units.

30. The method of claim 27, wherein the NK cell is an induced NK cell created from a precursor cell.

31. The method of claim 30, wherein the precursor cell is a hESC, hematopoietic stem cell, iPSC, and/or induced hematopoietic stem cell.

32. The method of any one of claims 1-31, wherein the NK cell comprises a transgene.

33. The method of claim 32, wherein the transgene encodes a chimeric antigen receptor (CAR), a T-cell receptor (TCR), a non-naturally occurring or naturally occurring variant of FcγRIII (CD16), an interleukin (e.g., interleukin 15 (IL-15), interleukin 15 receptor (IL-15R) or a variant thereof, interleukin 12 (IL-12), interleukin 21 (IL-21), interleukin 18 (IL-18), interleukin-12 receptor (IL-12R) or a variant thereof), a human leukocyte antigen (e.g., human leukocyte antigen G (HLA-G), human leukocyte antigen E (HLA-E)), leukocyte surface antigen cluster of differentiation CD47 (CD47), or any combination of two or more thereof.

34. The method of claim 33, wherein the NK cell comprises a transgenic CAR.

35. The method of claim 34, wherein the NK cell comprises more than one transgenic CAR.

36. The method of claim 34, wherein the transgenic CAR is a CD70 targeting CAR.

37. The method of claim 34, wherein the transgenic CAR is a TROP2 targeting CAR.

38. The method of claim 33, wherein the NK cell comprises a transgenic TCR.

39. The method of claim 38, wherein the transgenic TCR is an NY-ESO targeting TCR.

40. The method of any one of claims 1-39, wherein the NK cell comprises a mutation in an endogenous gene.

41. The method of claim 40, wherein the endogenous gene is an immunomodulatory gene.

42. The method of claim 40 or 41, wherein the endogenous gene is NKG2A, SIGLEC-7, LAG3, TIM3, CISH, FOXOl, TGFBR2, TIGIT, CD96, ADORA2, NR3C1, PD1, PDL-1, PDL-2, CD47, SIRPA, SHIP1, ADAM 17, RPS6, 4EBP1, CD25, CD40, IL21R, ICAM1, CD95, CD80, CD86, IL10R, TDAG8, CD5, CD7, SLAMF7, CD38, LAG3, TCR, beta2-microglobulin, HLA, CD73, GCR, CREM, ICER, CREB1, and/or CD39.

43. The method of any one of claims 1-42, wherein the NK cell is activated and/or expanded prior to deactivation.

44. The method of claim 43, wherein the NK cells are activated and/or expanded by culturing with a cell culture solution comprising universal Antigen Presenting Cells (uAPC), IL-2, IL-12, IL-15, and/or IL-18.

45. The method of claim 43 or 44, wherein the NK cells are activated and/or expanded for about 5 to about 20 days, about 8 to about 17 days, about 10 to about 15 days, about 12 days, about 13 days, or about 14 days prior to deactivation.

46. The method of any one of claims 1-45, wherein the deactivated NK cells are frozen and cryopreserved for any period of time.

47. The method of claim 46, wherein the deactivating agent is included in the cryopreservation media.

48. The method of claim 46, wherein the deactivating agent is washed off of the deactivated NK cells prior to cryopreservation.

49. The method of claim 48, wherein the deactivating agent is washed off using 0.5% HSA Plasma-Lyte A buffer.

50. A cryopreserved deactivated NK cell produced by the method of any one of claims 46-49.

51. A thawed deactivated cell produced by thawing the cryopreserved deactivated NK cell of claim 50.

52. The thawed deactivated NK cell of claim 51, wherein the thawed deactivated NK cell is washed to remove the deactivating agent.

53. The thawed deactivated NK cell of claim 51 or 52, wherein the thawed deactivated NK cell is reactivated in the absence of a deactivating agent, producing a reactivated NK cell.

54. The reactivated NK cell of claim 53, wherein the reactivated NK cell has improved survival rates relative to a non-deactivated thawed cryopreserved NK cell.

55. The reactivated NK cell of claim 53 or 54, wherein if the reactivated NK cell comprises a transgene, the transgene expression levels are not significantly decreased relative to a non-deactivated thawed cryopreserved NK cell.

56. The reactivated NK cell of claim 55, wherein the transgene expression levels are increased relative to a non-deactivated thawed cryopreserved NK cell.

57. The reactivated NK cell of any one of claims 53-56, wherein the reactivated NK cell has increased tumor cell killing rates following cryopreservation relative to a non-deactivated thawed cryopreserved NK cell.

58. A method of treating a subject in need thereof, comprising the step of administering to the subject a therapeutically effective amount of the reactivated NK cell of any one of claims 53-57.

59. The method of claim 58, wherein the subject has cancer.

60. The method of claim 59, wherein the cancer is hematological.

61. The method of claim 60, wherein the hematological cancer is myeloma.

62. The method of claim 59, wherein the cancer comprises a solid tumor.

63. The method of claim 62, wherein the cancer comprises ovarian cancer, pancreatic cancer, and/or brain cancer.

64. The method of claim 59, wherein the cancer is of hematopoietic origin.

65. The method of any one of claims 58-64, wherein the reactivated NK cells are allogeneic or autologous with respect to the subject.

66. The method of any one of claims 58-65, wherein the reactivated NK cells are allogeneic with respect to the subject.

67. The method of any one of claims 58-66, wherein the subject has an improved probability of survival relative to a subject not treated with an effective dose of a reactivated NK cell.


SEQUENCE LISTING (6 sequences)

SEQ ID NO: 1 - DNA, 18 bp, synthetic construct
  source 1..18: mol_type = other DNA
tgctgttgaggagctgga

SEQ ID NO: 2 - DNA, 18 bp, synthetic construct
  source 1..18: mol_type = other DNA
agcacaccaggcagagtt

SEQ ID NO: 3 - DNA, 18 bp, synthetic construct
  source 1..18: mol_type = other DNA
cggctgaggagcggaaga

SEQ ID NO: 4 - DNA, 18 bp, synthetic construct
  source 1..18: mol_type = other DNA
tggaggtgagcaatcccc

SEQ ID NO: 5 - DNA, 20 bp, synthetic construct
  source 1..20: mol_type = other DNA
ttaatacgactcactatagg

SEQ ID NO: 6 - DNA, 20 bp, synthetic construct
  source 1..20: mol_type = other DNA
gttttagagctagaaatagc